(19) United States
(12) Patent Application Publication
(10) Pub. No.: US 2023/0416846 A1
(43) Pub. Date: Dec. 28, 2023

(54) DIGITAL CRISPR-BASED METHOD FOR THE RAPID DETECTION AND ABSOLUTE QUANTIFICATION OF NUCLEIC ACIDS

(71) Applicants: MASSACHUSETTS INSTITUTE OF TECHNOLOGY, CambrIdge, MA (US); National University of Singapore, Singapore (SG)

(72) Inventors: Xiaolin Wu, Singapore (SG); Hanry Yu, Singapore (SG); Timothy Kuan-ta Lu, Cambridge, MA (US)

(73) Assignees: MASSACHUSETTS INSTITUTE OF TECHNOLOGY, Cambrodge, MA (US); National University of Singapore, Singapore (SG)

(21) Appl. No.: 18/251,277

(22) PCT Filed: Oct. 28, 2021
(86) PCT No.: PCT/SG2021/050661

§ 371 Date: May 1, 2023

(51) Int. Cl. C12Q 1/70; C12N 9/22

(52) U.S. Cl. CPC C12Q 1/70; C12N 9/22; G01N 2800/26; C12N 2310/20

(57) ABSTRACT

The present invention relates to a digital CRISPR-based method for detecting and quantitating target nucleic acids in a sample comprising: forming a mixture comprising sample nucleic acids; isothermal amplification reaction reagents for amplifying one or more target nucleic acid sequences; partitioning the mixture into a plurality of compartments; incubating the partitioned mixture at a temperature for isothermal amplification and Cas effector cleavage of an amplified DNA strand, detecting a signal from cleavage of the non-target sequence, thereby detecting the one or more target sequences in the sample, and determining the copy number of the target nucleic acid based on a Poisson distribution of the proportion of positive-to-negative compartments. The invention also relates to a method for detecting presence and/or of a disease in a subject, and a kit to quantitate nucleic acids in a sample.

### FIELD OF THE INVENTION

The present invention relates to digital CRISPR-based methods for detecting and quantitating target nucleic acids in a sample, a kit for use in such methods, and methods for detecting the presence and/or severity of disease in a subject.

### BACKGROUND OF THE INVENTION

DNA and RNA are usually used as detection targets to indicate the presence of a biological entity. Technologies for nucleic acid quantification are needed in diverse areas, ranging from biomedical research to clinical diagnostics to environmental protection. The widely used RT-qPCR, as a gold standard for COVID-19 diagnosis, offer advantages in speed and sensitivity but require precise thermal cycling and high PCR efficiency. Quantification via RT-qPCR relies on the use of external standards or references, and the results can be variable, with a 20-30% variability reported even within trained laboratories [Sedlak, R. H. and Jerome, K. R. Diagnostic microbiology and infectious disease 75: 1-4 (2013)]. Thus, an absolute quantification method with improved precision and accuracy is vital for virus research.

Digital PCR is increasingly used as a highly accurate and sensitive method for absolute quantification of nucleic acids [Salipante, S. J. and Jerome, K. R. Clinical chemistry 66: 117-123 (2020); Sedlak, R. H. and Jerome, K. R. Diagnostic microbiology and infectious disease 75: 1-4 (2013)]. In a digital PCR reaction, the PCR mixture is separated into thousands of individual reactions, resulting in either zero or one of the nucleic acid target molecules present in each partition. After independent PCR amplification and endpoint fluorescence detection of each partition, the copy number of the sample is determined based on the proportion of positive partitions. Since the PCR reaction in each partition proceeds independently, absolute quantification by digital PCR is more precise, more tolerant to inhibitors and overcomes poor amplification efficiency [Whale, A. S. et al. Nucleic acids research 40: e82-e82 (2012)]. The sensitivity and precision of digital PCR-based virus detection has been demonstrated in quantitative detection and viral load analysis of, for example, SARS-CoV-2-infected patient samples with a limit of detection (LOD) at ˜2 copies/reaction and fewer false negatives and fewer false positives compared with RT-PCR [Alteri, C. et al. PloS one 15: e0236311 (2020); Liu, X. et al. Emerging microbes & infections 9: 1175-1179 (2020); Suo, T. et al. Emerging microbes & infections 9(1): 1259-1268 (2020); Yu, F. et al. Clinical Infectious Diseases 71: 793-798 (2020)]. In addition to its application in viral diagnostics, digital PCR has also been successfully used in other areas of virus research, including the study of aerodynamic transmission of SARS-CoV-2 and in quantifying residual SARS-CoV-2 load in pulmonary tissues of a virus-negative patient by nasopharyngeal swab-qPCR test [Liu, Y. et al. Nature 582: 557-560 (2020); Yao, X.-H. et al. Cell research 30: 541-543 (2020)]. The main drawback of digital PCR, however; is the relatively long reaction time (˜4 hours) needed as a result of the 1-2° C./s ramp rate for efficient inter-partition heat transfer during thermal cycling, compared to that of qPCR which requires 1 hour. Reducing the reaction time of digital PCR is therefore crucial in enabling the adoption of the technology in rapid virus detection.

Isothermal amplification methods, which amplify the nucleic acid target molecule at a constant temperature and thereby reduce the reaction time have also been used in virus detection. These include methods that employ recombinase polymerase amplification (RPA) or loop-mediated isothermal amplification (LAMP) [Notomi, T. et al. Nucleic acids research 28: E63-E63 (2000); Piepenburg, O. et al. PLOS Biology 4: e204 (2006); Tomita, N. et al. Nature protocols 3: 877-882 (2008)]. More recently, innovative diagnostic methods using RNA-guided CRISPR/Cas system have been developed to detect nucleic acids. In the RNA-guided CRISPR/Cas system, Cas effectors such as Cas12a, Cas12b and Cas13a are exploited for their “collateral cleavage activity”: once the Cas protein finds and cleaves a specific DNA/RNA target, it binds and degrades other nonspecific DNA/RNA oligos such as fluorescently-tagged reporter oligos [Chen, J. S. et al. Science 360: 436-439 (2018); Gootenberg, J. S. et al. Science 356: 438-442 (2017); Li, S. Y. et al. Cell discovery 4: 20 (2018)]. By combining RPA- or LAMP-mediated isothermal amplification of the target molecule with the CRISPR/Cas biosensing system, methods such as SHERLOCK and DETECTR have successfully demonstrated the detection of dengue virus, human papillomavirus as well as SARS-CoV-2 in clinical samples [Broughton, J. P. et al. Nature biotechnology (2020); Ding, X. et al. Nature communications 11: 4711 (2020); Gootenberg, J. S. et al. Science 356: 438-442 (2017); Li, S. Y. et al. Cell discovery 4: 20 (2018)]. However, as CRISPR-based methods are not quantitative and require multiple manipulations between the amplification and detection steps, there remains a need for a quantitative, rapid and robust virus detection method.

There is a need for an improved molecular platform to enable rapid, visual and modular detection and quantification of nucleic acids and other target molecules.

### SUMMARY OF THE INVENTION

The present invention provides a nucleic acid detection and quantification system comprising: a CRISPR system comprising an effector protein and one or more guide RNAs designed to bind to corresponding target molecules; a Nucleic Acids-based masking construct; a sample partitioning step which subdivides the sample into a plurality of compartments, and optionally, nucleic acid amplification reagents to amplify target molecules in a sample. This method combines the advantages of quantitative digital PCR, rapid isothermal amplification and specific CRISPR detection into a one-pot reaction system which partitions the individual reactions into a plurality of small compartments in a high-density chip. In this study, we demonstrate a digital CRISPR method (also called RApid Digital Crispr Approach, RADICA) that allows for absolute quantification of nucleic acids at a constant temperature in one hour. We validated this method using DNA containing the N (nucleoprotein) gene of SARS-CoV-2, and showed a linear signal-to-input response of R² value>0.99. We further compared our digital CRISPR detection system against the traditional digital PCR method and show superior speed using the digital CRISPR system (1 h vs 4 h) while demonstrating comparable sensitivity and accuracy to that of traditional digital PCR. Further, we successfully used digital CRISPR in the absolute quantification of Epstein-Barr virus from human B cells, human adenovirus and herpes simplex virus (R² value>0.98) as well multiplex detection of several targets in a reaction. Together, our rapid and sensitive digital CRISPR method allows for accurate detection and absolute quantification of nucleic acids.

In a first aspect there is provided a method for detecting and quantitating target nucleic acids in a sample comprising:
a) forming a mixture comprising sample nucleic acids;
isothermal amplification reaction reagents for amplifying one or more target nucleic acid sequences;
a Cas12a, Cas12b, Cas13b or Cas14 effector, or a derivative thereof;
at least one guide polynucleotide comprising a DNA-targeting sequence, and designed to form a complex with the Cas effector; and
a nucleic acids-based masking construct comprising a non-target sequence,
b) partitioning the mixture into a plurality of compartments;
c) incubating the partitioned mixture at a temperature for isothermal amplification and Cas effector cleavage of an amplified DNA strand,
wherein the Cas effector exhibits collateral nuclease activity and cleaves the non-target sequence of the nucleic acids-based masking construct once activated by the target sequences; and
d) detecting a signal from cleavage of the non-target sequence, thereby detecting the one or more target sequences in the sample, and
e) determining the copy number of the target nucleic acid based on a Poisson distribution of the proportion of positive-to-negative compartments.

In some embodiments, the Cas effector is Cas12a or Cas12b.

In some embodiments, the method is used to detect and/or quantify a pathogen, gene expression, gene copy number variation or adventitious agents in a sample.

In some embodiments, the at least one guide polynucleotide is crRNA.

In some embodiments, the amplification is selected from the group comprising nucleic-acid sequence-based amplification, recombinase polymerase amplification, loop-mediated isothermal amplification, strand-displacement amplification, exonuclease III-assisted signal amplification, hybridization chain reaction, helicase-dependent amplification, isothermal circular strand displacement polymerization, multiple displacement amplification, primase-based whole genome amplification, rolling circle amplification and whole genome amplification.

In some embodiments, the amplification coupled with Cas12a is selected from the group comprising recombinase polymerase amplification, strand-displacement amplification, rolling circle amplification and multiple displacement amplification.

In some embodiments, the amplification coupled with Cas12b is selected from the group comprising loop-mediated isothermal amplification, helicase-dependent amplification, strand-displacement amplification and rolling circle amplification.

In some embodiments, the amplification coupled with Cas13 is selected from the group comprising nucleic-acid sequence-based amplification recombinase polymerase amplification and strand-displacement amplification.

In some embodiments, the amplification coupled with Cas14 is selected from the group comprising recombinase polymerase amplification, strand-displacement amplification and rolling circle amplification.

In some embodiments, the isothermal amplification is recombinase polymerase amplification or loop-mediated isothermal amplification.

In some embodiments, the masking construct suppresses generation of a detectable positive signal until cleaved, or masks a detectable positive signal until the masking construct is cleaved.

In some embodiments, the masking construct comprises a quenched fluorescent nucleic acids probe, such as a ssDNA probe, dsDNA or RNA probe.

In some embodiments, the target is DNA or RNA.

In some embodiments, the target is virus DNA or RNA.

In some embodiments, the virus is SARS-CoV-2 virus, human adenovirus (HAdV), herpes simplex virus (HSV) or Epstein-Barr virus (EBV).

In some embodiments, the partitioning is microfluidics-based, droplets-based or membrane-based, preferably chip-based.

In some embodiments, the mixture is partitioned into at least 1,000 compartments, preferably at least 10,000 compartments.

In some embodiments, the guide has a sequence comprising a mismatch to the one or more target sequences.

In some embodiments, the mismatch is up or downstream of a single nucleotide variation in the guide sequence.

In some embodiments, the isothermal amplification is recombinase polymerase amplification (RPA). A schematic of a recombinase polymerase amplification RADICA according to the invention is shown in FIG. 1.

In some embodiments, the RPA reaction is at 42° C. in step c).

In some embodiments, the Cas effector is Cas12a, In some embodiments, the Cas12a homolog is from Lachnospiraceae bacterium ND2006 (LbCas12a).

It was found that using Cas12a at 42° C. in step c), a 40 min incubation was enough for qualitative detection and 60 min incubation is enough for quantitative detection (Example 3).

In some embodiments, the isothermal amplification is a warm-start LAMP or RT-LAMP reaction.

In some embodiments, the warm-start RT-LAMP reaction is at 60° C. in step c).

In some embodiments, the Cas effector is Cas12b, which has been shown to be compatible with the one-pot RT-LAMP reaction with high sensitivity). A schematic of a warm-start RADICA according to the invention is shown in FIG. 24.

In some embodiments, the Cas effector is Cas12b from Alicyclobacillus acidiphilus (AapCas12b).

In some embodiments, WarmStart RTx reverse transcriptase, WarmStart DNA polymerase, and Cas12b/crRNA are combined. In some embodiments Bst 2.0 WarmStart DNA polymerase is used.

In some embodiments, the nucleic acids-based masking construct is a quenched fluorescent reporter comprising 5 thymine (T) bases. Advantageously, this masking construct shows high signal-to-noise ratio and reaction rate.

In some embodiments, taurine is added to the reaction mixture to improve reaction kinetics.

In some embodiments, 40 min incubation is enough for qualitative detection and 60 min incubation is enough for quantitative detection by warm-start RADICA.

In some embodiments, the isothermal amplification reaction is a multiplex reaction.

In some embodiments, the multiplex reaction detects a target nucleic acid and a human nucleic acid (DNA) control.

In some embodiments, the multiplex reaction detects a wildtype target nucleic acid and a variant or mutant thereof. For example, a multiplex reaction is used to detect wildtype SARS-CoV-2 N (or other) gene and a SARS-CoV-2 mutant variant thereof. The reaction may comprise multiplex primers to detect a wildtype target nucleic acid and at least one variant thereof, as exemplified in Examples 17 and 18 and FIGS. 45-48.

In some embodiments, the target nucleic acids are SARS-CoV-2, HAdV, HSV, or Epstein-Barr virus nucleic acids;
the isothermal amplification is:
a) recombinase polymerase amplification; the Cas effector is Cas12a; or
b) LAMP or RT-LAMP; the Cas effector is Cas12b;
the at least one guide polynucleotide is crRNA;
the mixture is partitioned into at least 1,000 compartments, preferably at least 10,000 compartments, and is chip-based; and the masking construct comprises a quenched fluorescent ssDNA probe.

In some embodiments, the amplification reaction is warm-start.

In a second aspect there is provided a method for detecting the presence and/or severity of a disease in a subject, comprising the steps of:
a) forming a mixture comprising a sample comprising nucleic acids from the subject;
isothermal amplification reaction reagents for amplifying one or more target disease nucleic acid sequences;
a Cas12a, Cas12b, Cas13b or Cas14 effector, or a variant thereof;
at least one guide polynucleotide comprising a DNA-targeting sequence, and designed to form a complex with the Cas effector; and
a nucleic acids-based masking construct comprising a non-target sequence,
b) partitioning the mixture into compartments;
c) incubating the partitioned mixture at a temperature for isothermal amplification and Cas effector cleavage of an amplified DNA strand, wherein the Cas effector exhibits collateral nuclease activity and cleaves the non-target sequence of the nucleic acids-based masking construct once activated by the target sequences;
d) detecting a signal from cleavage of the non-target sequence, thereby detecting the one or more target sequences in the sample;
e) determining the copy number of the target nucleic acid based on a Poisson distribution of the proportion of positive-to-negative compartments and comparing the number to a control value;
wherein positive compartments indicate the presence of disease in said subject, and wherein the copy number of the target nucleic acid indicates the severity of the disease in said subject.

In some embodiments, the disease is a pathogen infection.

In some embodiments, the disease is a virus infection.

In some embodiments, the Cas effector is Cas12a and/or Cas12b.

In some embodiments, the method is used to detect and/or quantify a pathogen, gene expression or gene copy number variation.

In some embodiments, the at least one guide polynucleotide is crRNA.

In some embodiments, the amplification is selected from the group comprising nucleic-acid sequence-based amplification, recombinase polymerase amplification, loop-mediated isothermal amplification, strand-displacement amplification, exonuclease III-assisted signal amplification, hybridization chain reaction, helicase-dependent amplification, isothermal circular strand displacement polymerization, multiple displacement amplification, primase-based whole genome amplification, rolling circle amplification and whole genome amplification.

In some embodiments, the isothermal amplification is recombinase polymerase amplification or loop-mediated isothermal amplification.

In some embodiments, the masking construct suppresses generation of a detectable positive signal until cleaved, or masks a detectable positive signal until the masking construct is cleaved.

In some embodiments, the masking construct comprises a quenched fluorescent nucleic acids probe.

In some embodiments, the target is DNA or RNA.

In some embodiments, the target is virus DNA or RNA.

In some embodiments, the virus is SARS-CoV-2, human adenovirus (HAdV), herpes simplex virus (HSV), or Epstein-Barr virus.

In some embodiments, the partitioning is microfluidics-based, droplets-based or membrane-based, preferably chip-based.

In some embodiments, the mixture is partitioned into at least 1,000 compartments.

In some embodiments, the isothermal amplification is a warm-start RT-LAMP reaction.

In some embodiments, the isothermal amplification reaction is a multiplex reaction.

In some embodiments, the method further comprises administering a treatment that is efficacious for the severity of the disease in said subject.

In a third aspect there is provided a kit to quantitate target nucleic acids in a sample comprising:
a) isothermal amplification reaction reagents for amplifying one or more target nucleic acid sequences;
b) a Cas12a, Cas12b, Cas13b or Cas14 effector, or a variant thereof;
c) at least one guide polynucleotide comprising a DNA-targeting sequence, and designed to form a complex with the Cas effector;
d) a nucleic acids-based masking construct comprising a non-target sequence, and
e) a partitioning device or substrate.

In some embodiments of the kit, the target nucleic acids are SARS-CoV-2, human adenovirus (HAdV), herpes simplex virus (HSV), or Epstein-Barr virus nucleic acids; the isothermal amplification reaction reagents are:
a) recombinase polymerase amplification reaction reagents; the Cas effector is Cas12a; or
b) warm-start RT-LAMP amplification reaction reagents; the Cas effector is Cas12b;
the at least one guide polynucleotide is crRNA; the partitioning device or substrate comprises at least 1,000 compartments and is chip-based; and the nucleic acids-based masking construct comprises at least one quenched fluorescent ssDNA probe.

In some embodiments, the partitioning device or substrate comprises at least 10,000 compartments.

### BRIEF DESCRIPTION OF FIGURES

FIG. 1A-B shows a schematic illustration of one design example of RADICA. (A) RADICA workflow. Typically, after the DNA/RNA extraction step, different kinds of samples can be used for the detection and quantification of various targets. The sample mixture containing nucleic acids, RPA reagents, and Cas12a-crRNA-FQ reporters is distributed randomly into thousands of partitions. In each partition, the nucleic acids target is amplified by RPA and detected by Cas12a-crRNA, resulting in a fluorescent signal in the partition. The proportion of positive-to-negative compartments is analyzed based on the endpoint fluorescence measurement, and the copy number of the target nucleic acids is calculated based on the Poisson distribution. (B) Illustration of RPA-Cas12a reaction in each positive partition. In each compartment containing the target molecule, RPA initiates from one DNA strand and subsequently exposes the crRNA-targeted ssDNA region on the other strand, due to the strand displacement of DNA polymerase. As the amplification proceeds, Cas12a cleaves the positive ssDNA strand, triggering its collateral cleavage activity, which in turn cleaves the proximal quenched fluorescent reporter (ssDNA-FQ reporter) to generate a fluorescence signal. At the same time, ongoing amplification of the other DNA strand exponentially amplifies the target DNA, triggering more Cas12a activation and increasing the fluorescence readout.

FIG. 2A-B shows Cas12a detection sensitivity of dsDNA dilution series without pre-amplification. (A) Kinetics of Cas12a-crRNA on dsDNA dilution series. (B) Detection sensitivity analysis of dsDNA dilution series with Cas12a-crRNA in 1 h. 50 nM Cas12-crRNA and 250 nM FQ-ssDNA reporter were incubated with dsDNA dilution series at 37° C. and fluorescence was monitored every 5 min.

FIG. 3A-B shows Cas12a increase the fluorescent signal of RPA reaction. The comparison of fluorescent signal of RPA (A) and Cas12a (B) in digital reaction. RPA signals was detected by SYTO82 fluorescent nucleic acid stain (exhibits orange fluorescence upon binding to nucleic acids, A) and Cas12a signals by FQ reporter (exhibits green fluorescence upon cutting by Cas12a, B) in digital detection. The x-axis represents fluorescence intensity of the partitions while the y-axis represents the frequency of the partitions. The left peak (low fluorescence level) on the fluorescence intensity histogram represents the negative partitions while the right peak (high fluorescence level) indicates the positive partitions.

FIG. 4A-B shows detection of dsDNA without preamplification at different Cas12a/crRNA concentrations. (A) 1 nM dsDNA incubated with 10-250 nM Cas12a-crRNA and 250 nM FQ ssDNA reporter. (B) 0.1 nM dsDNA incubated with 50-500 nM Cas12a-crRNA and 500 nM FQ ssDNA reporter.

FIG. 5A-D shows Cas12a bulk reactions with different FQ reporter concentrations. (A) Time course reaction of Cas12a with FQ reporters at concentrations ranging from 50 nM to 10,000 nM. X-axis indicates the reaction time; y-axis indicates the background-subtracted fluorescence signal. (B, C, D) Fluorescence signal of different concentrations of DNA obtained with FQ reporters at 500 nM (B), 10,000 nM (C), and 25,000 nM (D) after one hour reaction.

FIG. 6A-B shows RPA+Cas12a bulk reaction at different temperatures. (A, B) RPA+Cas12a one-pot reaction on serial dilutions of DNA at 42° C. (A) or 25° C. (B).

FIG. 7A-F shows optimization of RADICA (digital RPA-Cas12a). (A) Fluorescence signal of DNA and non-template control obtained with FQ reporters at concentrations ranging from 50 nM to 10,000 nM. (B) Histogram showing ratios of positive partitions on the chip with FQ reporters, at concentrations of 500 or 1000 nM, in the presence of target DNA (4 replicates for each FQ reporter concentration). (C) Fluorescence intensity of the negative partitions (background noise, low fluorescence) and positive partitions (positive signals, high fluorescence) on the chip obtained with FQ reporters at concentrations of 500 or 1000 nM. (D) RPA-Cas12a one-pot reaction of plasmid DNA at different temperatures (25° C., 37° C., and 42° C.). (E) Fluorescence intensity of the partitions on the chip at two time points. The x-axis represents fluorescence intensity while the y-axis represents the frequency of the partitions. The left peak (low fluorescence level) on the fluorescence intensity histogram represents the negative partitions while the right peak (high fluorescence level) indicates the positive partitions. As the CRISPR reaction proceeds, the fluorescence levels of the positive partitions increase and the right peak shifts further to the right. (F) The proportion of positive partitions at different time points of RADICA. Starting at 40 minutes, the fluorescence signal plateaus and the ratio of positive partitions reach a stable level.

FIG. 8 shows RADICA-based detection of different concentrations of SARS-CoV-2 N gene DNA. Fluorescence intensity histogram and scatter plot of the partitions on the chip for serial dilutions of DNA. Four dilutions of linearized plasmid DNA encoding the SARS-CoV-2 N gene (0.8, 127, 600, 1997 copies/A) and one non-template control (without plasmid DNA) were used as input DNA. The x-axis represents fluorescence intensity while the y-axis represents the frequency of the partitions. The left peak (low fluorescence level) on the fluorescence intensity histogram represents the negative partitions while the right peak (high fluorescence level) indicates the positive partitions. In the scatter plot, each dot represents one partition on the chip.

FIG. 9 shows a comparison of the absolute quantification of RADICA and dPCR. Each point represents one sample. The original linearized plasmid DNA concentration was measured by using dPCR and diluted to different concentrations (x-axis). The diluted DNA was then measured by using the RADICA. The calculated RADICA DNA concentrations are plotted on the y-axis.

FIG. 10A-D shows the effect of plasmid conformation on the accuracy of RADICA and dPCR. (A, B) The positive and negative partitions of RADICA (A) and dPCR (B) on detection of 179 copies/μL circular plasmids. (C, D) Comparison of the absolute quantification for linearized plasmid and circular plasmid of RADICA (C) and dPCR (D).

FIG. 11A-C shows specificity analysis for SARS-CoV-2. (A) Sequence alignment of the SARS-CoV-2 target region (N gene) and the corresponding regions on other human coronaviruses. (B) Time course reaction of RPA-Cas12a assay on SARS-CoV-2, SARS-CoV, and MERS-CoV N gene DNA target. The same concentration (25000 copies/A) of the N gene target from different coronaviruses was tested by the bulk RPA-Cas12a assay. (C) Specificity of RPA-Cas12a assay for detection of the SARS-CoV-2 N gene.

FIG. 12A-B shows RPA inhibition by human background DNA. (A) Results of RADICA reaction with target DNA and various amounts of human background DNA. Each dot represents one sample. (B) Comparison of the RADICA reaction with or without 1 ng/μL human background DNA.

FIG. 13A-E shows RADICA reaction with RNA. (A) Design of two-reverse-primers to increase the sensitivity. One reverse primer design only includes normal forward and reverse primer and two-reverse-primers design add a reverse primer 2 in addition to normal forward and reverse primer. (B, C) Bulk RPA-Cas12a reaction on RNA at different concentrations with normal one reverse primer design (B) or two-reverse-primers design (C). (D) Correlation of positive partition percentage of RADICA and target SARS-CoV-2 RNA copy number using normal primer design. (E) Comparison of RADICA's performance on normal one reverse primer design and two-reverse-primers design. 1400 copies/μL of RNA were processed using normal one reverse primer design and two-reverse-primers design. The ratio of positive partitions increased when using two reverse primers.

FIG. 14 shows the absolute quantification of RADICA on SARS-CoV-2 RNA based on Poisson distribution.

FIG. 15A-D shows RADICA reaction on SARS-CoV-2 RNA N gene (N0 and N1 regions). (A, B) Correlation of positive partition percentage of RADICA and target SARS-CoV-2 RNA copy number using two-reverse-primers' design. (C, D) Sensitivity analysis of RADICA in direct detecting SARS-CoV-2 RNA using two-reverse-primers' design.

FIG. 16 shows primer and crRNA design for RADICA assay specific for Epstein-Barr virus (EBV). Epstein-Barr virus (EBV), also known as human herpesvirus 4 (HHV4), is a member of the herpes virus family. Consensus sequences of the EBV genome EBNA-1 and BamHIW regions were used as a template for the primer and crRNA design. Consensus sequence (SEQ ID NO: 308 and 325), HHV4 strain YCCEL1 (AP015016.1; SEQ ID NO: 309 and 326), HHV4 strain GD1 (AY961628.3; SEQ ID NO: 310 and 327), HHV4 strain HKNPC1 (JQ009376.2; SEQ ID NO: 311 and 328), HHV4 strain Akata (KC207813.1; SEQ ID NO: 312 and 329), HHV4 strain Mutu (KC207814.1; SEQ ID NO: 313 and 330), HHV4 strain K4123-Mi (KC440851.1; SEQ ID NO: 314 and 331), HHV4 strain K4123-MiEBV (KCC440852.1; SEQ ID NO: 315 and 332), HHV4 strain C666-1 (KC617875.1; SEQ ID NO: 316), HHV4 strain M81 (KF373730.1; SEQ ID NO: 317 and 333), HHV4 strain Raji (KF717093.1; SEQ ID NO: 318 and 334), HHV4 strain GC1 (KP735248.1; SEQ ID NO: 319 and 335), HHV4 strain Jijoye (LN827800.1; SEQ ID NO: 320 and 336), HHV4 (NC007605.1; SEQ ID NO: 321 and 337), HHV4 (NC009334.1; SEQ ID NO: 322 and 338), EBV strain B95-8 (V01555.2; SEQ ID NO: 323 and 339).

FIG. 17 shows absolute quantification of Epstein-Barr virus (EBV) by RADICA. Fluorescence intensity histogram, scatter plot, and position plot of the partitions on the chip on serially-diluted EBV DNA.

FIG. 18A-B shows absolute quantification of Epstein-Barr virus (EBV) by RADICA. (A, B) A comparison of the absolute quantification values obtained from RADICA and dPCR using various concentrations of EBV DNA. (A) is targeting EBV EBNA-1 target, (B) is targeting EBV BamHIW target.

FIG. 19A-B shows validation of RADICA on clinical samples. (A) Correlation between qPCR- and dPCR-based EBV BamHI-W target detection in 79 serum samples. (B) Correlation between qPCR- and RADICA-based EBV BamHI-W target detection in 79 serum samples.

FIG. 20 shows a Box and Whisker plot of the EBV viral load in 22 NPC patients at the point of initial diagnosis, one year after treatment, and at the point of recurrence.

FIG. 21 shows a Heat map displaying the measured EBV DNA copy number in each of the 22 NPC patients at the point of initial diagnosis, one year after treatment, and at the point of recurrence by qPCR, dPCR and RADICA.

FIG. 22 shows a primer and crRNA screen for RADICA on human genomic DNA. Different primer/crRNA set targeting human RNAseP gene were screened using RPA-Cas12a bulk reaction. The primer/crRNA set with the highest speed was selected for the following RADICA experiment.

FIG. 23 shows a RADICA test on human genomic DNA. Human genomic DNA under different dilution were tested using RADICA targeting the RNAseP gene.

FIG. 24A-C shows a schematic illustration of another form of RADICA (digital RT-LAMP-Cas12b). (A) Overview of the RADICA (digital RT-LAMP-Cas12b) process. Nucleic acids (DNA and RNA) are extracted from different types of samples, then mixed with RT-LAMP and Cas12b/crRNA/FQ reporter mix. The reaction mixtures can be subdivided into thousands of partitions by digital chips, followed by incubation at 60° C. for 1 h. The partitions containing the target yield a much higher fluorescent signal than the partitions without targets, and the end-point results are detected by a fluorescent detector to calculate the proportions of positive partitions. (B) Reactions in the single positive partitions. The DNA/RNA, RT-LAMP, and Cas12b/crRNA/FQ reporters are mixed in a one-pot format in each partition. The target DNA/RNA can be amplified by RT-LAMP into looped structures. Because the amplified targets are complementary with crRNA, they bind to a Cas12b/crRNA complex, triggering the trans cleavage of Cas12b to cut the FQ reporters, which in turn results in a fluorescent signal. (C) Schematic concept of the bulk RT-LAMP-Cas12b assay.

FIG. 25A-B shows the FQ reporter base composition's effect on bulk RT-LAMP-Cas12b reaction. (A) Bulk RT-LAMP-Cas12b reactions with different FQ reporter sequences at the same length were monitored at 60° C. Fluorescent signals of reactions with target RNA and non-template control were compared. (B) Comparison of bulk RT-LAMP-Cas12b reaction under FQ reporter with different base compositions (poly A, poly T, poly C, poly G, and poly AT) by detecting 20 copies/μL synthetic SARS-CoV-2 RNA. Error bars represent the standard deviation (s.d.) from at least three replicates (n≥3). SARS-CoV-2 N gene was used as a target in the Figures above.

FIG. 26A-B shows the FQ reporter length's effect on bulk RT-LAMP-Cas12b reaction. (A) Bulk RT-LAMP-Cas12b reactions with different FQ reporter lengths were monitored at 60° C. Fluorescent signals of reactions with target RNA and non-template control were compared. (B) Comparison of bulk RT-LAMP-Cas12b reaction with different lengths of FQ reporter (5 nt to 20 nt) by detecting 20 copies/μL synthetic SARS-CoV-2 RNA. Error bars represent the standard deviation (s.d.) from at least three replicates (n≥3). SARS-CoV-2 N gene was used as a target in the Figures above.

FIG. 27A-B shows the effect of taurine on bulk RT-LAMP-Cas12b reaction. (A) Bulk RT-LAMP-Cas12b reactions with different taurine were monitored at 60° C. Fluorescent signals of reactions with target RNA and non-template control were compared and signal-to-noise ratios were calculated as Y-axis. At least three replicates were used for each concentration and error bars indicate the standard deviation of the replicates. (B) The effect of taurine on the bulk RT-LAMP-Cas12b reaction by detecting 20 copies/μL synthetic SARS-CoV-2 RNA. Error bars represent the standard deviation (s.d.) from at least three replicates (n≥3). SARS-CoV-2 N gene was used as a target in the Figures above.

FIG. 28A-B shows concentrations of WarmStart RTx reverse transcriptase's effect on RT-LAMP-Cas12b reaction. (A) Bulk RT-LAMP-Cas12b reactions with different reverse transcriptase concentrations were monitored at 60° C. Fluorescent signals of reactions with target RNA and non-template control were compared. (B) RADICA reactions with different reverse transcriptase concentrations. The upper panels are end-point fluorescence images from QIAcuity software while the lower panels show the proportion of positive partitions in a total number of around 26,000 partitions for each replicate (partition volume ˜0.91 nL). SARS-CoV-2 N gene was used as a target in the Figures above.

FIG. 29 shows an end-point fluorescent image to optimize the concentrations of WarmStart RTx reverse transcriptase's effect on RADICA. SARS-CoV-2 N gene was used as a target in the Figures above.

FIG. 30A-B shows the effect of concentration of Bst 2.0 WarmStart DNA polymerase on LAMP-Cas12b reaction. (A) Bulk RT-LAMP-Cas12b reactions with different polymerase concentrations were monitored at 60° C. Fluorescent signals of reactions with target RNA and non-template control were compared. (B) RADICA reactions with different polymerase concentrations. The upper panels are end-point fluorescence images from QIAcuity software while the lower panels show the proportion of positive partitions in a total number of around 26,000 partitions for each replicate (partition volume ˜0.91 nL). SARS-CoV-2 N gene was used as a target in the figures above.

FIG. 31 shows an end-point fluorescent image to optimize the concentrations of WarmStart DNA polymerase's effect on RADICA. SARS-CoV-2 N gene was used as a target in the figures above.

FIG. 32A-B shows RADICA reactions with different Cas12b concentrations. (A) RADICA reactions with different Cas12b concentrations. (B) End-point fluorescent image to optimize the concentrations of Cas12b's effect on RADICA. SARS-CoV-2 N gene was used as a target in the figures above.

FIG. 33A-B shows time-course reactions of RT-LAMP-Cas12b. (A, B) Real-time reactions of bulk RT-LAMP-Cas12b with target RNA or NTC. LAMP signal was detected by SYTO82 nucleic acid stain (exhibits orange fluorescence upon binding to nucleic acids, A) and Cas12b signals by FQ reporter (exhibits green fluorescence upon cutting by Cas12b, B). Error bars represent the standard deviation (s.d.) from at least three replicates (n≥3). n.s., not significant (student's t test). SARS-CoV-2 N gene was used as a target in the figures above.

FIG. 34 shows time-course reactions of RADICA (digital LAMP-Cas12b). End-point fluorescence results of the RADICA at different incubation time at 60° C.

FIG. 35 shows time-course reactions of RADICA (digital LAMP-Cas12b). Comparison of positive partition percentages of RADICA with different reaction times at 60° C.

FIG. 36A-E shows quantitative detection of nucleic acids by RADICA (digital LAMP-Cas12b) on Clarity digital chip. (A) Specificity of RADICA reaction. Primers and crRNA targeting SARS-CoV-2 were used to test the RADICA reaction for SARS-CoV-2, SARS-CoV, and MERS-CoV sequences with a human DNA background. The scatter plot represents a total number of around 10,000 partitions for one sample (partition volume ˜1.336 nL). Three replicates were performed with similar results and one representation was shown here. (B, C) RADICA with different concentrations of DNA target using a Clarity digital chip. (D, E) RADICA with different concentrations of RNA target using a Clarity digital chip. For figures B and D, the upper panels are representative end-point fluorescence images while the lower panels show the scatter plot representing a total number of around 10,000 partitions (partition volume ˜1.336 nL) for one sample. Figures C and E represent the correlation and linear relationship between the input target concentrations and positive partition percentages. Error bars represent the standard deviation (s.d.) from at least three replicates (n≥3).

FIG. 37A-D shows quantitative detection of nucleic acids by RADICA (digital LAMP-Cas12b) on QIAcuity digital nanoplate. (A, B) RADICA with different concentrations of DNA target using QIAcuity digital nanoplate. (C, D) RADICA with different concentrations of RNA target using QIAcuity digital nanoplate. For figures A and C, the upper panels are representative end-point fluorescence images while the lower panels show the scatter plot representing a total number of around 26,000 partitions (partition volume ˜0.91 nL) for one sample. B and D represent the correlation and linear relationship between the input target concentrations and positive partition percentages. Error bars represent the standard deviation (s.d.) from at least three replicates (n≥3).

FIG. 38A-D shows the copy number result of RADICA according to Poisson distribution. (A, B) RADICA with different concentrations of DNA or RNA target using a Clarity digital chip. (C, D) RADICA with different concentrations of DNA or RNA target using QIAcuity digital nanoplate. Error bars represent the standard deviation (s.d.) from at least three replicates (n≥3).

FIG. 39A-E shows a comparison of RADICA with other detection methods. The detection sensitivity of RT-qPCR (A), RT-dPCR (B), Bulk RT-LAMP-Cas12b assay (C), RADICA by Clarity digital chip (D), and RADICA by QIAcuity digital nanoplate (E) using different concentrations of SARS-CoV-2 RNA as the target with the background of 1 ng/μL human genomic DNA. Error bars represent the standard deviation (s.d.) from at least three replicates (n≥3).

FIG. 40A-B shows a comparison of RADICA with other detection methods. (A) Quantitative results of RT-qPCR, RT-dPCR, Bulk RT-LAMP-Cas12b assay, RADICA by Clarity digital chip and RADICA by QIAcuity digital nanoplate in detecting SARS-CoV-2 RNA at various concentrations. Heat map displaying the average measured RNA concentration by the four methods using different concentrations of target RNA input (n≥3 for each method at each concentration). (B) Comparison of the effects of reaction inhibitors on RT-qPCR, RT-dPCR, Bulk RT-LAMP-Cas12b assay, and RADICA. 1250 copies/μL of SARS-CoV-2 synthetic RNA were tested in the absent/present of different inhibitors using the different methods indicated above. For figure B, error bars represent the standard deviation (s.d.) from at least three replicates (n≥3).

FIG. 41 shows a comparison of inhibitory effect of heparin on bulk RT-LAMP-Cas12b and RADICA. 1250 copies/μL of SARS-CoV-2 synthetic RNA were tested in the absent/present of heparin using the above two methods. Error bars represent the standard deviation (s.d.) from at least three replicates (n≥3).

FIG. 42A-D shows a screen of LAMP primer and crRNA for human adenovirus (A, C) and herpes simplex virus (B, D). (A, B) Cq values of LAMP were used to screen the LAMP primer. (Faster primers result in lower Cq). (C, D) Fluorescences of LAMP-Cas12b after 60 min reaction were used to screen the crRNA. (Faster crRNAs result in high fluorescent signal).

FIG. 43A-B shows RADICA with different concentrations of human adenovirus (A) and herpes simplex virus (B) DNA using QIAcuity digital nanoplate. The scatter plot representing a total number of around 26,000 partitions (partition volume ˜0.91 nL) for one sample.

FIG. 44A-B shows quantitative detection of human adenovirus and herpes simplex virus by RADICA. DNA extracted from human adenovirus (A) and herpes simplex virus (B) were tested by RADICA in 1 ng/μL human genomic DNA background. Error bars represent the standard deviation (s.d.) from at least three replicates (n≥3).

FIG. 45 shows multiplex RADICA on SARS-CoV-2 and human DNA control. Multiplex RADICA reaction detecting different concentrations of SARS-CoV-2 N gene (FAM, RT-LAMP-Cas12b based) and constant concentration of human ACTB gene (ROX, probe-based LAMP) using QIAcuity digital nanoplate. The images represent the end-point fluorescence signal of RADICA.

FIG. 46 shows multiplex RADICA on SARS-CoV-2 and human DNA control. Multiplex RADICA reaction detecting different concentrations of SARS-CoV-2 N gene (FAM, RT-LAMP-Cas12b based) and constant concentration of human ACTB gene (ROX, probe-based LAMP) using QIAcuity digital nanoplate. The images represent the 2D Scatter plot. X-axis: ROX signal represents the human ACTB gene. Y-axis: FAM signal represents the SARS-CoV-2 N gene.

FIG. 47A-B shows a quantitative result of Multiplex RADICA on SARS-CoV-2 and human DNA control. RADICA reaction detecting different concentration of SARS-CoV-2 N gene (FAM, RT-LAMP-Cas12b based) and constant concentration of human ACTB gene (ROX, probe-based LAMP) using QIAcuity digital nanoplate. (A) The scatter plot representing a total number of around 26,000 partitions (partition volume ˜0.91 nL) for one sample. (B) The correlation and linear relationship between the input target concentrations and the concentrations measured by RADICA.

FIG. 48 shows multiplex RADICA on SARS-CoV-2 wildtype and mutant. RT-LAMP-Cas12a primer-crRNA set (FAM) targeting SARS-CoV-2 N gene (cover both wildtype and mutant), RT-LAMP primer/probe set (CY5) targeting SARS-CoV-2 alpha mutant S gene and RT-LAMP primer/probe set (ROX) targeting SARS-CoV-2 beta mutant S gene were used in RADICA assay.

FIG. 49 shows a scatter plot of multiplex RADICA on SARS-CoV-2 (3 fluorescence channels) in a constant human genomic DNA background (1 fluorescence channel). RT-LAMP-Cas12a primer-crRNA set targeting SARS-CoV-2 N gene (FAM channel), RT-LAMP primer-probe sets targeting SARS-CoV-2 E gene (HEX channel), ORF1ab gene (CY5 channel) and human ACTB gene (ROX channel) were used in this multiplex RADICA reaction to detect serial dilutions of SARS-CoV-2 RNA in a constant human genomic DNA background.

FIG. 50 shows quantitative multiplex RADICA on SARS-CoV-2 (3 fluorescence channels) in a constant human genomic DNA background (1 fluorescence channel). RT-LAMP-Cas12a primer-crRNA set targeting SARS-CoV-2 N gene (FAM channel), RT-LAMP primer-probe sets targeting SARS-CoV-2 E gene (HEX channel), ORF1ab gene (CY5 channel) and human ACTB gene (ROX channel) were used in this multiplex RADICA reaction to detect serial dilutions of SARS-CoV-2 RNA in a constant human genomic DNA background.

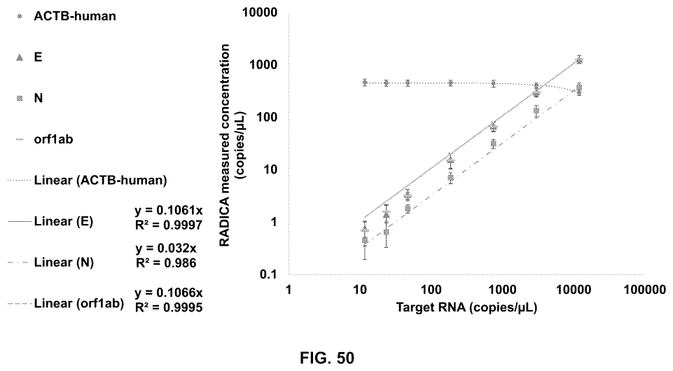

### DETAILED DESCRIPTION OF THE INVENTION

Bibliographic references mentioned in the present specification are for convenience listed in the form of a list of references and added at the end of the Examples. The whole content of such bibliographic references is herein incorporated by reference.

### Definitions

Unless defined otherwise, all technical and scientific terms used herein have the same meaning as is commonly understood by one of skill in the art to which the invention belongs. Certain terms employed in the specification, examples and appended claims are collected here for convenience.

It must be noted that as used herein and in the appended claims, the singular forms “a,” “an,” and “the” include plural reference unless the context clearly dictates otherwise. Thus, for example, a reference to “a target sequence” includes a plurality of such target sequences, and a reference to “an enzyme” is a reference to one or more enzymes and equivalents thereof known to those skilled in the art, and so forth.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “oligonucleotide”, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art. The recognition nanostructure may comprise an inverter oligonucleotide.

The term “sample,” as used herein, is used in its broadest sense. For example, a biological sample suspected of containing human adenovirus, HSV, EBV or SARS-CoV-2 genome sequences may comprise a bodily fluid; an extract from a cell, chromosome, organelle, or membrane isolated from a cell; a cell; genomic DNA, RNA, or cDNA (in solution or bound to a solid support); a tissue; a tissue print; and the like.

It would be understood that oligonucleotides used in the present invention may be structurally and/or chemically modified to, for example, prolong their activity in samples potentially containing nucleases, during performance of methods of the invention, or to improve shelf-life in a kit. Thus, the aptamer and/or inverter and/or signaling nanostructure or any oligonucleotide primers or probes used according to the invention may be chemically modified. In some embodiments, said structural and/or chemical modifications include the addition of tags, such as fluorescent tags, radioactive tags, biotin, a 5′ tail, the addition of phosphorothioate (PS) bonds, 2′-O-Methyl modifications and/or phosphoramidite C3 Spacers during synthesis.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### EXAMPLES

Standard molecular biology techniques known in the art and not specifically described were generally followed as described in Green and Sambrook, Molecular Cloning: A Laboratory Manual, Cold Spring Harbour Laboratory, New York (2012).

### Example 1

### Materials and Methods

### Materials:

The sequences of primers, crRNA, and FQ reporters were synthesised by Integrated DNA Technologies. Plasmids containing the N gene from each virus genomes (SARS-CoV-2, SARS-CoV, and MERS-CoV) were purchased from Integrated DNA Technologies. The synthetic RNA covering 99.9% of the bases of the SARS-CoV-2 viral genome was purchased from Twist Bioscience (Genbank ID: MN908947.3). The DNA and RNA concentrations were measured by dPCR or RT-dPCR assay. TwistAmp® Basic were from TwistDx. EnGen® Lba Cas12a were from New England Biolabs. Bst 2.0 WarmStart polymerase, WarmStart RTx Reverse Transcriptase and RNase Inhibitor were from New England Biolabs. Cas12b was from Magigen Biotechnology. Clarity JN solution and Clarity digital chip were from JN Medsys. QIAcuity digital nanoplates were from QIAGEN. The USCDC N2 assay for SARS-CoV-2 detection was from Integrated DNA Technologies. TaqMan™ Fast Virus 1-Step Master Mix was from Applied Biosystems.

crRNA Preparation for Cas12a:

Constructs were ordered as DNA from Integrated ssDNA Technologies with an appended T7 promoter sequence. crRNA ssDNA was annealed to a short T7 primer (T7-3G IVT primer [Kellner, M. J. et al. Nature protocols 14: 2986-3012 (2019)] or T7-Cas12scaffold-F [Lucia, C. et al. bioRxiv 2020.2002.2029.971127 (2020)] and treated with fill-in PCR (Platinum™ SuperFi II PCR Master Mix) to generate the DNA templates. These DNA were used as DNA templates to synthesize crRNA using the HiScribe™ T7 High Yield RNA Synthesis Kit (New England Biolabs) according to published protocols [Kellner, M. J. et al. Nature protocols 14: 2986-3012 (2019); Lucia, C. et al. bioRxiv 2020.2002.2029.971127 (2020)]. The synthesized crRNA was purified using Monarch® RNA Cleanup Kit (New England Biolabs) after treatment with DNase I (RNase-free, New England Biolabs), Thermolabile Exonuclease I (New England Biolabs), and T5 Exonuclease (New England Biolabs).

crRNA Preparation for Cas12b:

The DNA template for crRNA synthesis was obtained by first annealing the Cas12b crRNA universal scaffold oligo (DNA oligo) with the respective DNA oligo (containing the target region and the region complementary to Cas12b crRNA scaffold and filling in both ends using Platinum™ SuperFi II PCR Master Mix. The resulted DNA was used as a template for crRNA synthesis using HiScribe™ T7 High Yield RNA Synthesis Kit (New England Biolabs). The DNA template was then removed by DNase I (RNase-free, New England Biolabs), Thermolabile Exonuclease I (New England Biolabs), and T5 Exonuclease (New England Biolabs), and the crRNA was purified using Monarch® RNA Cleanup Kit (New England Biolabs).

### RADICA (Digital RPA+Cas12a) Quantification on Clarity Digital Chip:

The one-pot reaction combining RPA-DNA amplification and Cas12a detection was performed as follows: 300 nM forward primer, 300 nM reverse primer, 500 nM FQ reporter, 1×RPA rehydration buffer containing 1×RPA Pellet (TwistDx), 200 nM EnGen® Lba Cas12a (New England Biolabs), 200 nM crRNA, were prepared followed by adding various amounts of DNA input, and 14 mM magnesium acetate. When RNA was used as a target, 300 nM reverse primer 2 was used with 10 U/μL PhotoScript Reverse transcriptase (New England Biolabs) or 10 U/μL SuperScript™ IV Reverse Transcriptase (Invitrogen) and 0.5 U/μL RNase H (Invitrogen or New England Biolabs), as indicated. Commercial chips for sample partitioning and matched fluorescence reader for endpoint detection were used in RADICA. The RADICA reaction was prepared by adding 1× Clarity™ JN solution (JN Medsys) to the RPA-Cas12a bulk reactions stated above. To prevent spontaneous target amplification by RPA at room temperature, the RPA-CRISPR reaction was prepared without the addition of Mg2+, which is required for the polymerase activity. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### RADICA (Digital LAMP+Cas12b) Quantification on Clarity Digital Chip:

The DNA/RNA target samples were mixed with 1.6 μM FIP primers, 1.6 μM BIP primers, 0.2 μM F3 primers, 0.2 μM B3 primers, 0.4 μM LoopF primers, 0.4 μM LoopB primers, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, and 50 nM crRNA in 1× isothermal amplification buffer, unless otherwise indicated. RADICA reaction was prepared by adding 1× Clarity JN solution to the RT-LAMP-Cas12b bulk reactions described above and partitioned on Clarity digital chip (˜1.336 nL partition volume, ˜10,000 partitions per reaction). 15 μL reaction mixtures were loaded onto the digital chip followed by treatment with the Clarity sealing enhancer and sealing with 230 μL Clarity sealing fluid. The tube containing the digital chip was warmed in a water bath at 60° C. for 1 hour, unless otherwise indicated. After incubation, the end-point fluorescence in the 10,000 partitions was detected by Clarity Reader. Using Clarity software, the threshold was determined based on the florescence distribution of the partitions and positive partition percentages and input nucleic acids concentration were then calculated based on the threshold.

### RADICA (Digital LAMP+Cas12b) Quantification on QIAcuity Digital Nanoplate:

The DNA/RNA target samples were mixed with 1.6 μM FIP primers, 1.6 μM BIP primers, 0.2 μM F3 primers, 0.2 μM B3 primers, 0.4 μM LoopF primers, 0.4 μM LoopB primers, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, and 50 nM crRNA in 1× isothermal amplification buffer, unless otherwise indicated. The RADICA reaction was prepared by adding 250 nM Cyanine 680 succinimidyl ester (biotium) as reference dye to the RT-LAMP-Cas12b bulk reactions stated above and partitioned on QIAcuity digital nanoplates (˜0.91 nL partition volume, 26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

### Example 2

### Digital CRISPR Method Design: Digital RPA-Cas12a

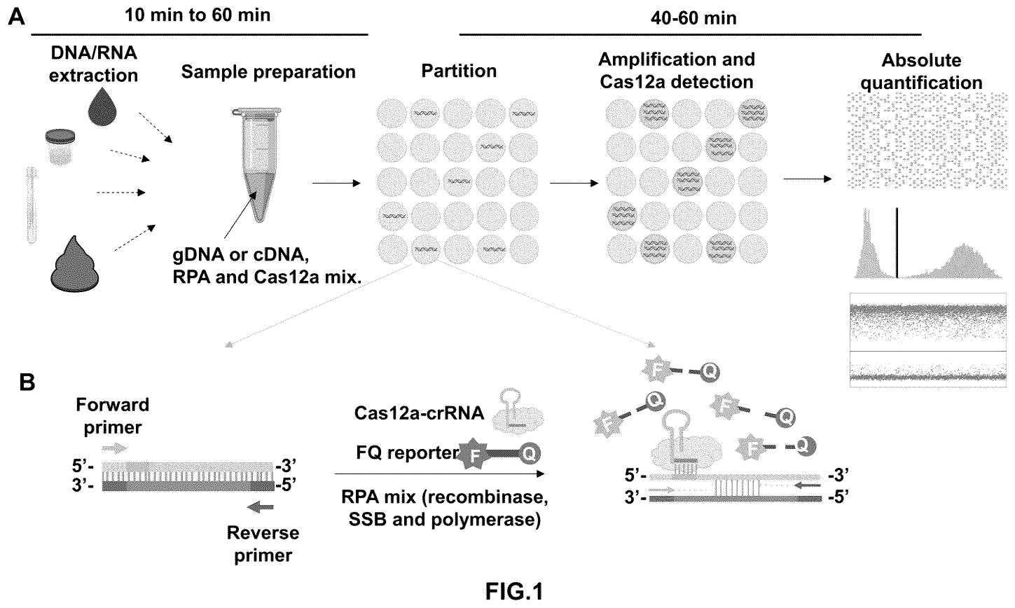

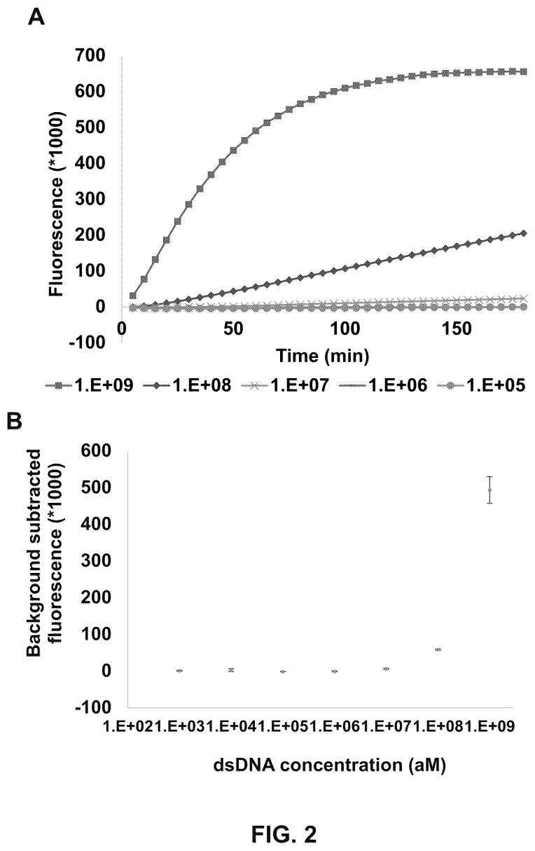

One example of RADICA design schematic is illustrated in FIG. 1A. Each CRISPR-based reaction mix is sub-divided into 10,000 partitions on the chip, resulting in zero or one target molecule in each compartment with an average partition volume of 1.336 nL. The copy number of target nucleic acids is calculated based on the proportion of positive-to-negative compartments, allowing for absolute quantification of the sample (FIG. 1A). We first optimized the bulk CRISPR reaction to achieve a one-copy-per-1.336 nL partition detection sensitivity on the chip. This is equivalent to femtomolar detection sensitivity in a bulk reaction. We selected Cas12a homolog from Lachnospiraceae bacterium ND2006 (LbCas12a) as it showed the highest signal-to-noise ratio relative to other Cas12a homologs [Li, S. Y. et al. Cell discovery 4: 20 (2018)]. To test if RADICA could detect DNA with femtomolar sensitivity without pre-amplification, we incubated serially-diluted double-stranded DNA (dsDNA) with LbCas12a, CRISPR RNA (crRNA) and a FQ reporter (quenched fluorescent DNA). The detection sensitivity of the CRISPR-based method without pre-amplification in a bulk reaction was found to be 100 pM (FIG. 2), which did not meet the femtomolar sensitivity requirement of RADICA.

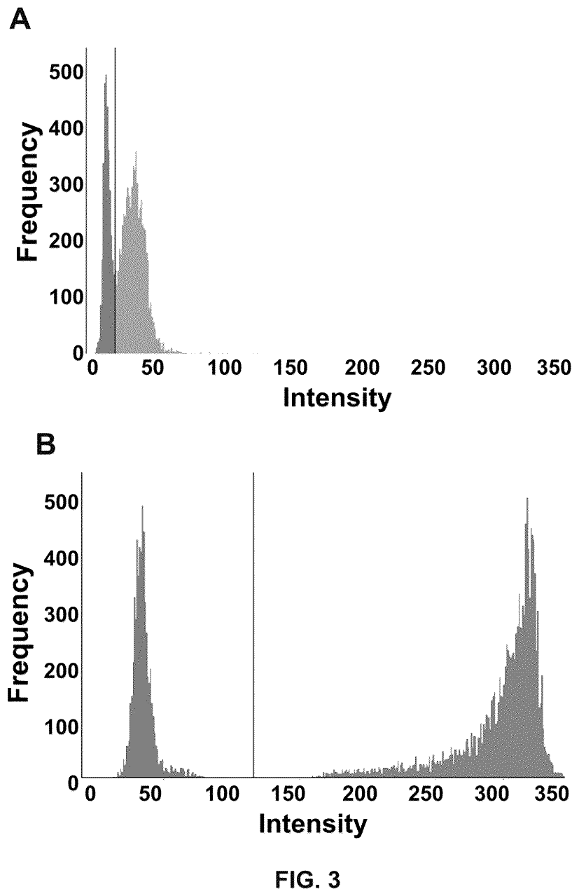

To increase the detection sensitivity, we added an isothermal amplification step using RPA, whose reaction temperature (25° C. to 42° C.) is compatible with that of Cas12a (25° C. to 48° C.). To avoid Cas12a-mediated cleavage of the target molecule before amplification, we designed crRNA to target single-stranded DNA (ssDNA) that is generated only after the amplification of the target molecule. This allowed for a one-step digital RPA-CRISPR absolute quantification method that eliminates multiple operations inherent in two-step CRISPR-based detection methods such as SHERLOCK, HOLMES and DETECTR [Chen, J. S. et al. Science 360: 436-439 (2018); Gootenberg, J. S. et al. Science 356: 438-442 (2017); Li, S. Y. et al. Cell discovery 4: 20 (2018)]. It is easier to design ssDNA-targeting crRNA than traditional dsDNA-targeting crRNA, because the nuclease activity of Cas12a in ssDNA is independent of the presence of a protospacer adjacent motif (PAM) [Li, S. Y. et al. Cell research 28: 491-493 (2018)]. We showed that Cas12a increased the signal to noise ratio of the partitions as it further amplifies the fluorescent signals in the positive partitions (FIG. 3).

### Example 3

### RADICA (Digital RPA-Cas12a) Method Optimization

Primers and crRNAs specific for dsDNA containing the SARS-CoV-2 N (nucleoprotein) gene were designed as described previously [Ding, X. et al. Nature communications 11: 4711 (2020)]. The target regions overlap those of the China CDC assay (N gene region) with some modification to meet the primer and crRNA design requirements. The method was optimized with the primers and crRNA shown in Table 1.

TABLE 1
Primers and crRNA used in RADICA optimization

| Name | Sequence | Application |
|---|---|---|
| ssDNA-FQ reporter | /56-FAM/TTATT/3IABKFQ/ | Cas12a FQ |
| | | reporter |
| N-RNA-F | gaaatTAATACGACTCACTATAgggA | Amplify N gene on |
| SEQ ID NO: 1 | TGTCTGATAATGGACCCCAAAAT | plasmids |
| N-RNA-R | gaaatTTAGGCCTGAGTTGAGTCA | Amplify N gene on |
| SEQ ID NO: 2 | GCACT | plasmids |
| CHNCDC-geneN-F | GGGGAACTTCTCCTGCTAGAAT | dPCR Primer for N |
| SEQ ID NO: 3 | | gene |
| CHNCDC-geneN-R | CAGACATTTTGCTCTCAAGCTG | dPCR Primer for N |
| SEQ ID NO: 4 | | gene |
| CHNCDC-geneN-P | /56- | dPCR Probe for N |
| SEQ ID NO: 5 | FAM/TTGCTGCTG/ZEN/CTTGACA | gene |
| | GATT/3IABKFQ/ | |
| N-N-RPA-F | AACTCCAGGCAGCAGTAGGGGAA | CRISPR Primer |
| SEQ ID NO: 6 | CTT | for N gene |
| N-N-RPA-R | CCTTTACCAGACATTTTGCTCTCA | CRISPR Primer |
| SEQ ID NO: 7 | AG | for N gene |
| N-N-crRNA-IVT | aatctgtcaagcagcagATCTACACTTA | crRNA template |
| SEQ ID NO: 8 | GTAGAAATTACCCTATAGTGAGTC | for N gene, pair |
| | GTATTAATTTC | with T7-3G IVT |
| | | primer, also used |
| | | as crRNA-dsDNA |
| | | template |
| N-AIOD-F | AGGCAGCAGTAGGGGAACTTCTC | RADICA Primer |
| SEQ ID NO: 9 | CTGCTAGAAT | for N gene, N1 |
| | | region* |
| N-AIOD-R | TTGGCCTTTACCAGACATTTTGCT | RADICA Primer |
| SEQ ID NO: 10 | CTCAAGCTG | for N gene, N1 |
| | | region* |
| N-AIOD-crRNA-IVT-F | ggctggcaatggcggtgatgATCTACACT | crRNA template |
| SEQ ID NO: 11 | TAGTAGAAATTACCCTATAGTGAG | for N gene, pair |
| | TCGTATTAATTTC | with T7-3G IVT |
| | | primer, also used |
| | | as crRNA-ssDNA1 |
| | | template |
| T7-3G IVT primer | GAAATTAATACGACTCACTATAGG | Universal primer |
| SEQ ID NO: 12 | G | used for crRNA |
| | | synthesis |

*Ding, X. et al., Nature Communications 11:4711 (2020).

### Methods:

Cas12a Bulk Assay without Preamplification:

Unless otherwise indicated, 50 nM EnGen® Lba Cas12a (New England Biolabs), 50 nM crRNA, and 250 nM FQ reporter were incubated with dsDNA dilution series in NEB buffer 2.1 at 37° C., and fluorescence signals were measured every 5 min.

### Preparation of DNA Targets:

SARS-CoV-2 N gene-containing G-Block dsDNA, SARS-CoV-2, SARS-CoV, and MERS N gene-containing plasmids were purchased from Integrated DNA Technologies. The SARS-CoV-2 N gene-containing plasmid (IDT) was linearized using FastDigest Scal (Thermo Scientific) and then used as DNA targets. The SARS-CoV-2 N gene-containing plasmid was used as a template to amplify the N gene using primer N-RNA-F/N-RNA-R by Platinum™ SuperFi II PCR Master Mix (Invitrogen). The PCR product was purified by QIAquick PCR Purification Kit (QIAGEN) and used as RNA synthesis templates.

### Synthetic RNA Target:

Since N-RNA-F has a T7 promoter sequence, the amplified DNA using N-RNA-F/R primer will contain a T7 promoter upstream of gene N. The T7 tagged N gene dsDNA was transcribed into SARS-CoV-2 RNA using HiScribe™ T7 High Yield RNA Synthesis Kit (New England Biolabs) according to the manufacturer's protocol. The synthesized RNA (N gene) was purified using the Monarch® RNA Cleanup Kit (New England Biolabs) after treatment with DNase I (RNase-free, New England Biolabs). The synthetic RNA covering 99.9% of the bases of the SARS-CoV-2 viral genome were purchased from Twist Bioscience (Genbank ID: MN908947.3).

### RPA-Cas12a Bulk Assay:

The one-pot reaction combining RPA-DNA amplification and Cas12a detection was performed as follows: 300 nM forward primer, 300 nM reverse primer, 500 nM FQ reporter, 1×RPA rehydration buffer containing 1×RPA Pellet (TwistDx), 200 nM EnGen® Lba Cas12a (New England Biolabs), 200 nM crRNA, were prepared followed by adding various amounts of DNA input, and 14 mM magnesium acetate. When detecting RPA signal is needed, 250 mM SYTO-82 fluorescent nucleic acids stain was added into the reaction. The reaction mixture was incubated at 42° C. unless otherwise indicated and fluorescence kinetics were monitored every 1 min.

### RADICA Optimization:

Commercial chips for sample partitioning and matched fluorescence reader for endpoint detection were used in RADICA. The RADICA reaction was prepared by adding 1× Clarity™ JN solution (JN Medsys) to the RPA-Cas12a bulk reactions stated above. To prevent spontaneous target amplification by RPA at room temperature, the RPA-CRISPR reaction was prepared without the addition of Mg2+, which is required for the polymerase activity. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### Results:

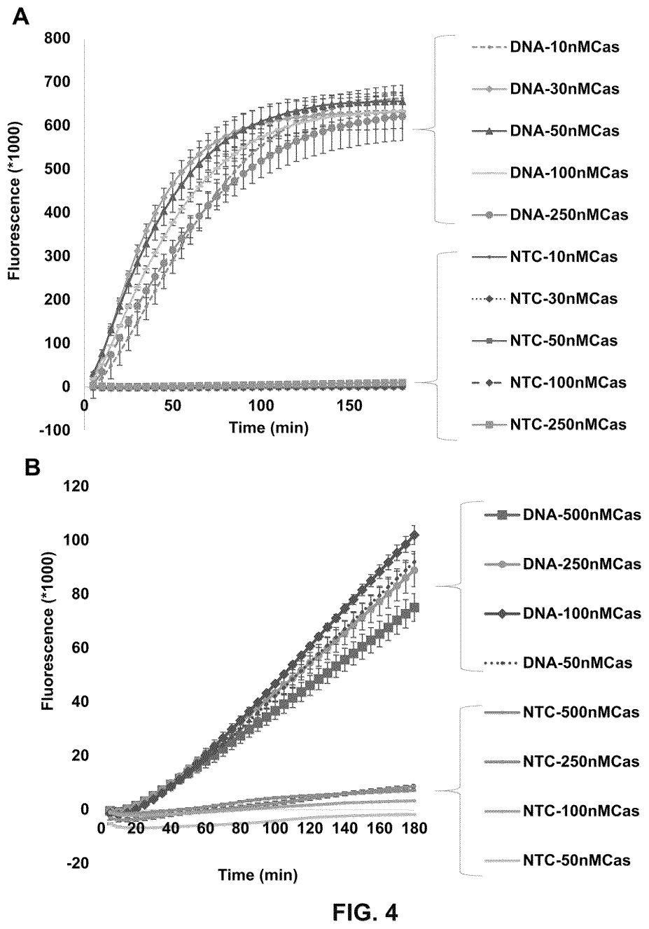

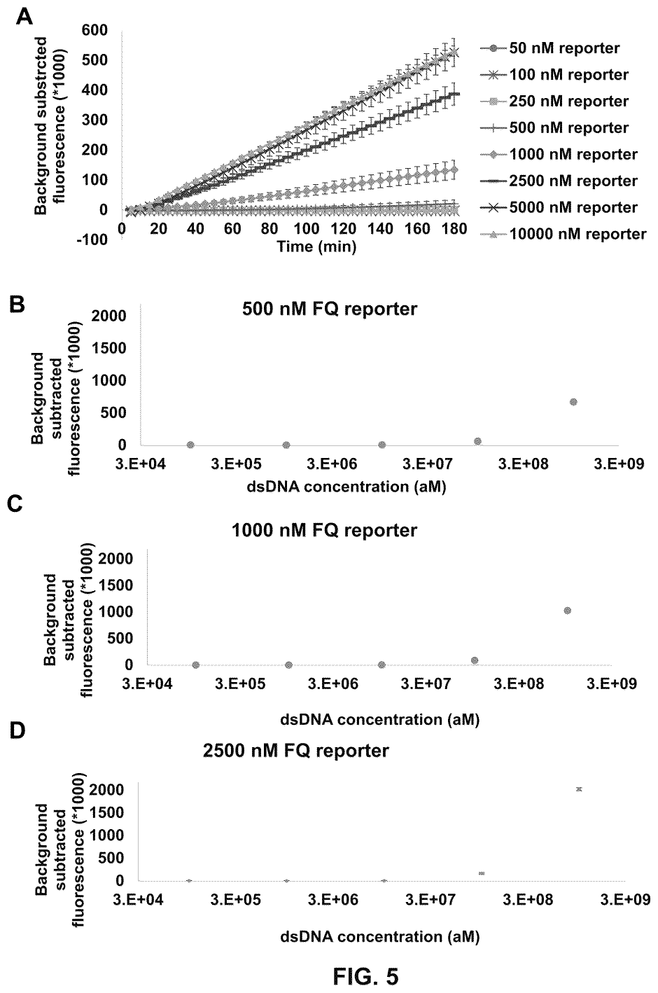

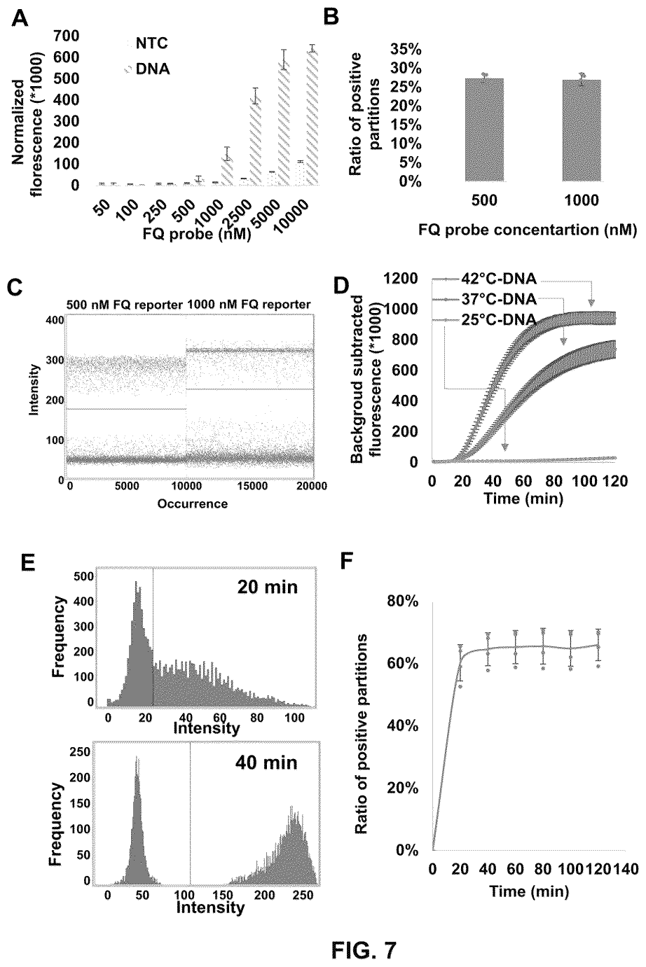

When a constant amount of dsDNA was used as a target in bulk reaction, 50 nM to 250 nM Cas12a/crRNA concentration has no influence on the fluorescence intensities and reaction rates (FIG. 4). However, fluorescence intensities of both target and negative control increased with increasing amounts of FQ reporter (from 250 nM to 10 μM) (FIG. 5, FIG. 7A). To improve the signal to noise ratio of RADICA, we tested different FQ reporter concentrations in independent digital CRISPR reactions in the presence of target DNA, and measured the fluorescence in the dPCR fluorescence reader. In the presence of the same target DNA, proportions of the positive partitions were comparable regardless of the FQ reporter concentration used (FIG. 7B). Only background noise and positive signals generated in the reaction with 500 nM FQ reporter concentration can be clearly separated, while the reactions containing 1000 nM FQ reporter concentration yielded higher background noises, which are difficult to be separated from the positive signals (FIG. 7C). We therefore used 500 nM FQ reporter concentrations to achieve high signal-to-noise ratios for the subsequent experiments.

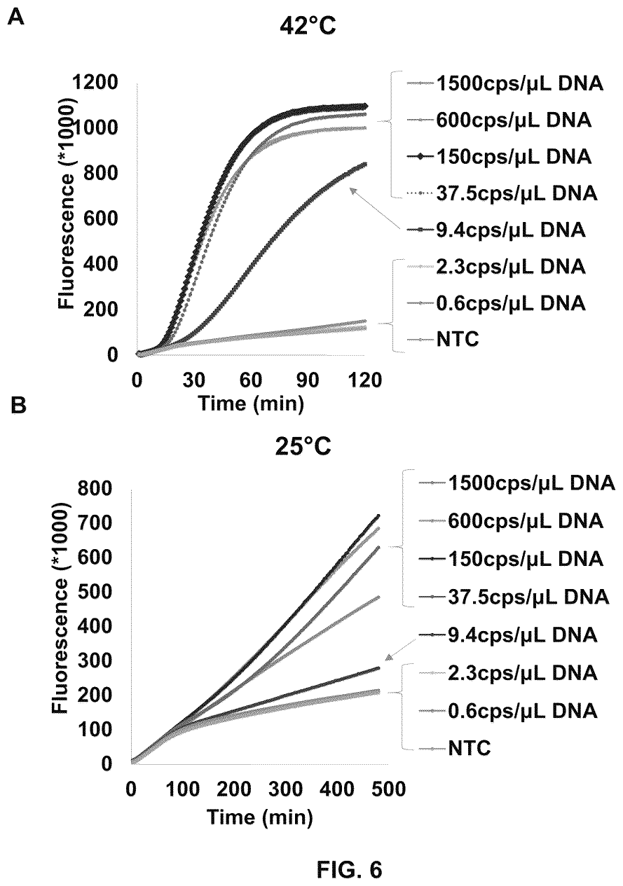

We combined the RPA and Cas12a reactions in a one-pot reaction. We performed the bulk reaction at 25° C., 37° C., and 42° C., which fall within the reaction temperature ranges of RPA (25° C. to 42° C.) and Cas12a (25° C. to 48° C.). With serial dilutions of plasmid DNA, the reaction proceeded at 25° C. and 42° C., with a limit of detection of 9.4 copies/μL (FIG. 6). At the reaction was significantly slower, with lower positive signals and higher background than the reaction performed at 42° C. (FIG. 6). We assessed the effect of different temperatures (25° C., 37° C., 42° C.) on reactions containing a constant amount of plasmid DNA (37.5 copies/μL). Higher temperatures accelerated the reaction (FIG. 7D). 42° C. is the optimal temperature for the RPA-Cas12a reaction.

We next investigated the earliest time that the reaction completes in all the partitions. The reaction proceeded quickly with an increase in fluorescence signals detected in some compartments at 20 min, but with a low signal-to-noise ratio at this time point (FIG. 7E). Two distinct peaks, indicating negative (left) and positive (right) partitions, were detected at 40 min with a good baseline separation (FIG. 7E). Analyzing the ratio of positive partitions on the chip at different time points revealed that the number of positive partitions reached a plateau after 40 min for all four replicates, suggesting that 40 min was the earliest time that the reactions in all the partitions have completed (FIG. 7F). To ensure all the micro-reactions are completed, all subsequent experiments were therefore performed for 60 min.

### Example 4

### Absolute Quantification of SARS-CoV-2 DNA Using RADICA (Digital RPA-Cas12a)

We characterized the performance of RADICA in detecting and quantifying SARS-CoV-2 and compared it to that of dPCR. Linearized plasmids containing the SARS-CoV-2 N gene were serially diluted and used as the target DNA in the aforementioned optimized RADICA or dPCR reactions. The method was optimized with the primers and crRNA shown in Table 2 for SARS-CoV-2 DNA detection.

TABLE 2
Primers and crRNA used in SARS-CoV-2 DNA detection

| Name | Sequence | Application |
|---|---|---|
| ssDNA-FQ reporter | /56-FAM/TTATT/3IABKFQ/ | Cas12a FQ |
| | | reporter |
| N-RNA-F | gaaatTAATACGACTCACTATAgggA | Amplify N gene on |
| SEQ ID NO: 1 | TGTCTGATAATGGACCCCAAAAT | plasmids |
| N-RNA-R | gaaatTTAGGCCTGAGTTGAGTCA | Amplify N gene on |
| SEQ ID NO: 2 | GCACT | plasmids |
| CHNCDC-geneN-F | GGGGAACTTCTCCTGCTAGAAT | dPCR Primer for N |
| SEQ ID NO: 3 | | gene |
| CHNCDC-geneN-R | CAGACATTTTGCTCTCAAGCTG | dPCR Primer for N |
| SEQ ID NO: 4 | | gene |
| CHNCDC-geneN-P | /56- | dPCR Probe for N |
| SEQ ID NO: 5 | FAM/TTGCTGCTG/ZEN/CTTGACA | gene |
| | GATT/3IABKFQ/ | |
| N-N-RPA-F | AACTCCAGGCAGCAGTAGGGGAA | CRISPR Primer |
| SEQ ID NO: 6 | CTT | for N gene |
| N-N-RPA-R | CCTTTACCAGACATTTTGCTCTCA | CRISPR Primer |
| SEQ ID NO: 7 | AG | for N gene |
| N-N-crRNA-IVT | aatctgtcaagcagcagATCTACACTTA | crRNA template |
| SEQ ID NO: 8 | GTAGAAATTACCCTATAGTGAGTC | for N gene, pair |
| | GTATTAATTTC | with T7-3G IVT |
| | | primer, also used |
| | | as crRNA-dsDNA |
| | | template |
| N-AIOD-F | AGGCAGCAGTAGGGGAACTTCTC | RADICA Primer |
| SEQ ID NO: 9 | CTGCTAGAAT | for N gene, N1 |
| | | region* |
| N-AIOD-R | TTGGCCTTTACCAGACATTTTGCT | RADICA Primer |
| SEQ ID NO: 10 | CTCAAGCTG | for N gene, N1 |
| | | region* |
| N-AIOD-crRNA-IVT-F | ggctggcaatggcggtgatgATCTACACT | crRNA template |
| SEQ ID NO: 11 | TAGTAGAAATTACCCTATAGTGAG | for N gene, pair |
| | TCGTATTAATTTC | with T7-3G IVT |
| | | primer, also used |
| | | as crRNA-ssDNA1 |
| | | template |
| T7-3G IVT primer | GAAATTAATACGACTCACTATAGG | Universal primer |
| SEQ ID NO: 12 | G | used for crRNA |
| | | synthesis |

*Ding, X. et al., Nature Communications 11:4711 (2020).

### Methods:

Primer and crRNA Design for RADICA Targeting (SARS-CoV-2):

SARS-CoV-2 primers and crRNA were designed based on previously published papers [Ding, X. et al. Nature communications 11: 4711 (2020)] or 264 SARS-CoV-2 genome sequences from GISAID [Shu, Y. and McCauley, J. Eurosurveillance 22: 30494 (2017)]. Other human-related coronavirus sequences were downloaded from NCBI. UGENE software was used to analyze and align viral genomes (MUSCLE or Kalign). Consensus sequences (threshold: 90%) of 264 SARS-CoV-2 genomes, 328 SARS-CoV, 572 MERS-CoV, 70 Human-CoV-229E genomes, 48 Human-CoV-HKU1 genomes, 71 Human-CoV-NL63, and 178 Human-CoV-0C43 were exported separately from UGENE and used for specificity analysis.

### SARS-CoV-2 N Gene Quantification by Digital PCR:

The G-block dsDNA, plasmid, dsDNA and RNA concentrations were quantified by dPCR. Serial dilutions of targets were mixed with 500 nM CHNCDC-geneN-F, 500 nM CHNCDC-geneN-R, 250 nM CHNCDC-geneN-P, 1× TaqMann™ Fast Virus 1-Step Master Mix (for RNA, Applied Biosystems) or TaqMann™ Fast Advanced Master Mix (for DNA, Applied Biosystems), 1× Clarity™ JN solution (JN Medsys). For RNA samples, the reaction mixture was incubated at 55° C. 5 min before partitioning the reaction mix on Clarity™ autoloader. Then the reaction partitions were sealed with the Clarity™ Sealing Enhancer and 230 μL Clarity™ Sealing Fluid, followed by thermal cycling using the following parameters: 95° C. for 15 min (one cycle), 95° C. 50 s and 56° C. 90 s (40 cycles, ramp rate=1° C./s), 70° C. 5 min. The endpoint fluorescence of the partitions was detected using Clarity™ Reader and the final DNA copy numbers were analyzed by Clarity™ software.

### RADICA Quantification of SARS-CoV-2:

The RADICA reaction mix for SARS-CoV-2 DNA was prepared by mixing 300 nM forward primer (N-AIOD-F), 300 nM reverse primer (N-AIOD-R), 500 nM FQ reporter, 1× RPA rehydration buffer containing 1×RPA Pellet (TwistDx), 200 nM EnGen® Lba Cas12a (New England Biolabs), 200 nM crRNA (synthesized by N-AIOD-crRNA-IVT-F and T7-3G IVT primer), 1× Clarity™ JN solution (JN Medsys), followed by adding various amounts of DNA input, and 14 mM magnesium acetate. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### Results:

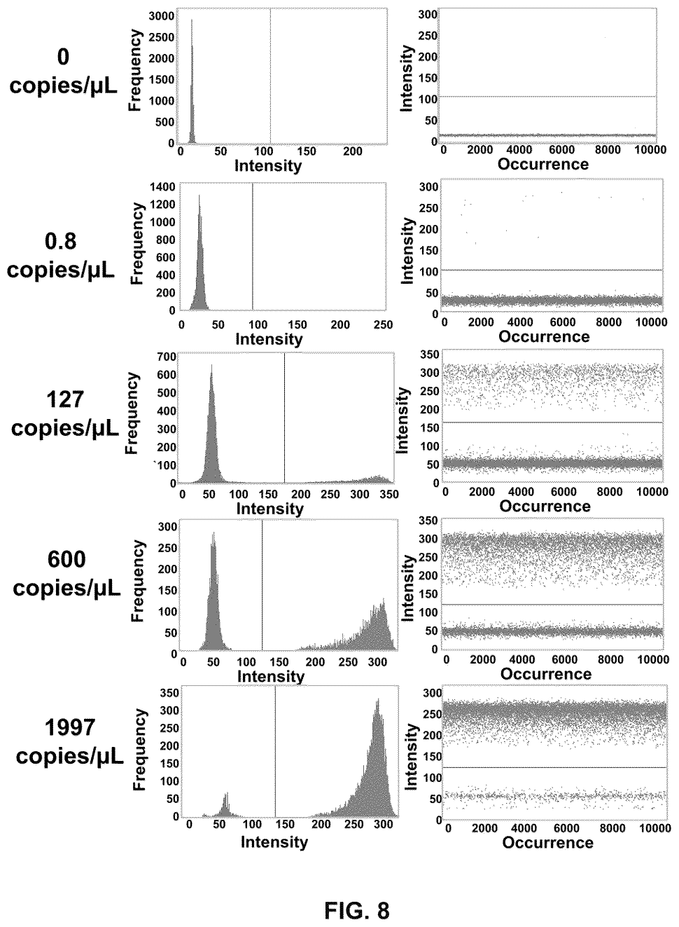

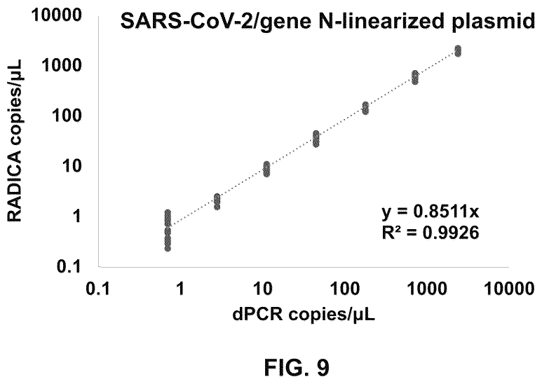

A proportional increase in the number of positive partitions with increasing concentrations of the target DNA was detected by RADICA (FIG. 8), indicating the analytical linearity of RADICA over three orders of magnitude. To test the robustness and reproducibility of RADICA, we performed at least ten independent RADICA reactions on different days. The inter-day coefficient of variation (CV) was ≤15% except for the lowest dilution (0.6 copies/μL), indicating the lower limit of quantification (LLoQ) of this method is 2.2 copies/μL of the viral genome (fulfils ≤15% CV criterion). The limit of blank (LoB) is 0.413 copies/μL, which is half of the calculated lower limit of detection (LLoD), 0.897 copies/μL. To assess the accuracy of nucleic acids detection for RADICA compared to dPCR, we plotted the DNA concentrations measured by RADICA against the corresponding DNA concentrations obtained by dPCR. Linear regression analysis revealed an R² value of above 0.99 across a dynamic range from 0.6 to 2027 copies/μL, indicating that RADICA showed strong linear correlations with dPCR (FIG. 9). These data highlight the high sensitivity, accuracy and precision of RADICA for the absolute quantification of nucleic acids.

### Example 5

### Accuracy Analysis of RADICA (Digital RPA-Cas12a)-Based Quantification on Circular Plasmid

### Methods:

The RADICA reaction mix for SARS-CoV-2 DNA was prepared by mixing 300 nM forward primer (N-AIOD-F), 300 nM reverse primer (N-AIOD-R), 500 nM FQ reporter, 1×RPA rehydration buffer containing 1×RPA Pellet (TwistDx), 200 nM EnGen® Lba Cas12a (New England Biolabs), 200 nM crRNA (synthesized by N-AIOD-crRNA-IVT-F and T7-3G IVT primer), 1× Clarity™ JN solution (JN Medsys), followed by adding various amounts of circular plasmid or linearized plasmid, and 14 mM magnesium acetate. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### Results:

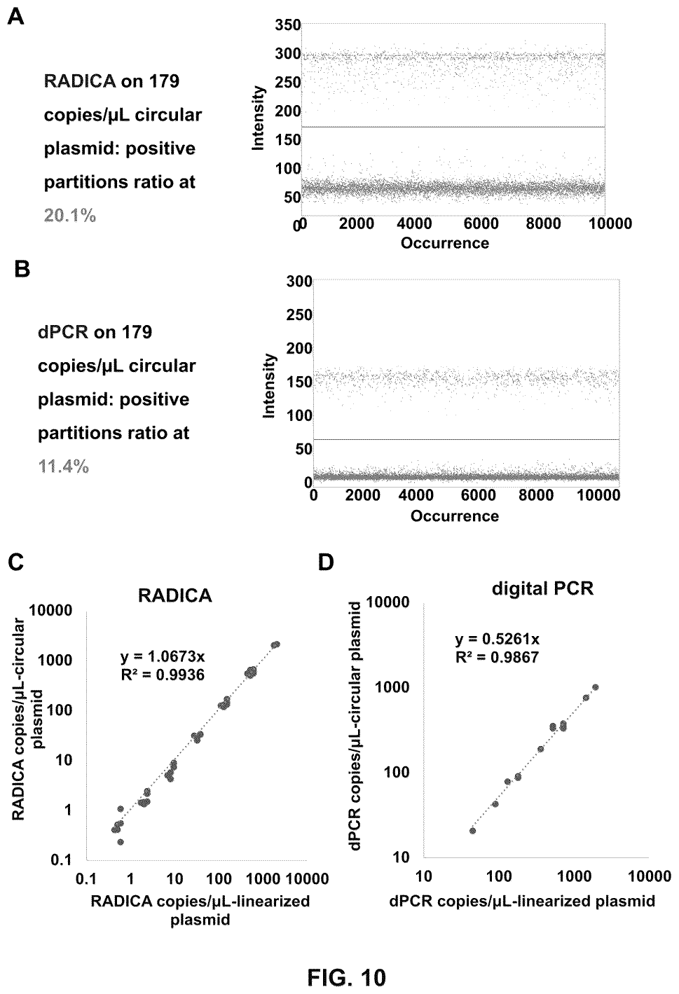

Plasmids are routinely used as reference DNA or standards; and conformational changes in supercoiled DNA can have a profound effect on PCR-based quantification. Single-molecule amplification of non-linearized plasmids was unsuccessful in a PCR-based study, resulting in an underestimation for circular plasmid quantification in some dPCR machines [Dong, L. et al. Scientific reports 5: 13174 (2015)].To test whether plasmid conformation affects the accuracy of RADICA, undigested plasmids containing SARS-CoV-2 N gene were serially diluted and used for digital PCR or RADICA reactions. Concentrations of non-linearized plasmids measured by dPCR were half of those detected for linearized plasmids (FIG. 10D), indicating that the accuracy of dPCR is influenced by plasmid conformation as previously reported [Dong, L. et al. Scientific reports 5: 13174 (2015)]. Compared to dPCR, RADICA showed a higher amplification efficiency of supercoiled plasmid DNA, as evidenced by the higher positive compartments' ratio (FIGS. 10A and 10B). RADICA concentrations of non-linearized plasmids were highly concordant with those of linearized plasmids (FIG. 10C), indicating that the accuracy of RADICA is not affected by plasmid conformation.

### Example 6

### Specificity Analysis of RADICA (Digital RPA-Cas12a)-Based Detection

### Methods:

The specificity of RADICA is analysed by 300 nM forward primer (N-AIOD-F), 300 nM reverse primer (N-AIOD-R), 500 nM FQ reporter, lx RPA rehydration buffer containing 1×RPA Pellet (TwistDx), 200 nM EnGen® Lba Cas12a (New England Biolabs), 200 nM crRNA (synthesized by N-AIOD-crRNA-IVT-F and T7-3G IVT primer), followed by adding plasmid containing the SARS-CoV-2 or SARS-CoV or MERS-CoV gene, and 14 mM magnesium acetate. Monitor the fluorescent signal of the reaction at 42° C.

### Results:

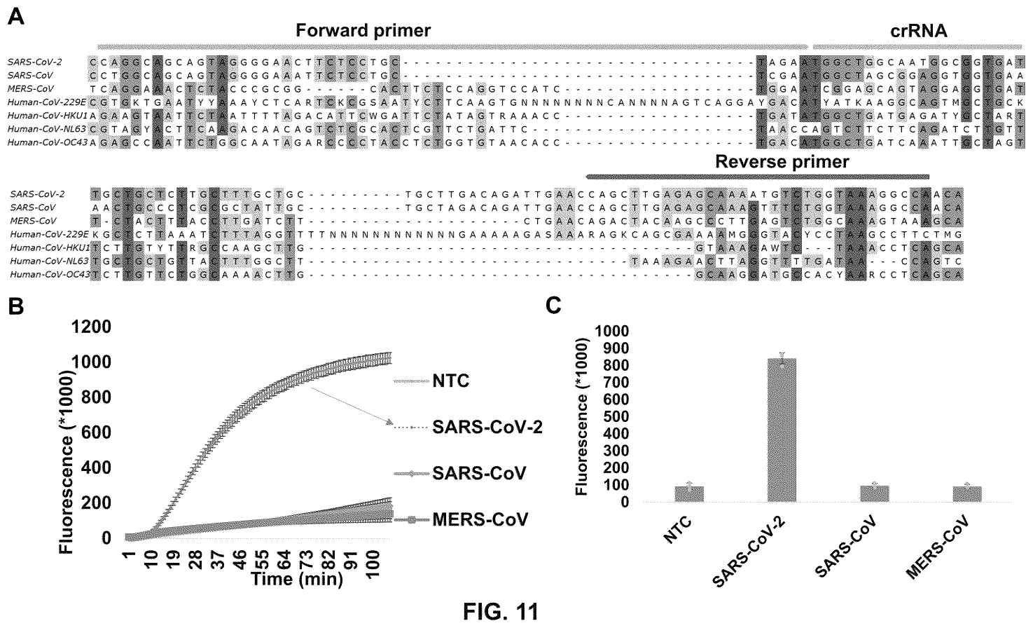

Primer and crRNA designs are key in determining the specificity of CRISPR-based nucleic acids detection. RPA tolerates up to nine nucleotide base-pair mismatches across primer and probe binding sites [Li, J. et al. The Analyst 144: 31-67 (2018)]. To specifically detect SARS-CoV-2 with RADICA, primers and crRNAs would have to specifically bind the SARS-CoV-2 target DNA but not the DNA of other related coronaviruses. We analyzed the binding sites of the primers and crRNAs that were originally designed based on the consensus sequence of the genomes of 264 SARS-CoV-2 strains, available on the GISAID database [Ding, X. et al. Nature communications 11: 4711 (2020); Shu, Y. and McCauley, J. Eurosurveillance 22: 30494 (2017)]. These consensus sequences were aligned with the corresponding regions of SARS-CoV-2-related beta coronaviruses, such as SARS-CoV, MERS-CoV, and human coronaviruses Human-CoV 229E/HKU1/NL63/OC43. No cross-binding regions were observed with the SARS-CoV-2-related coronaviruses analyzed (FIG. 11A). Although the five base-pair mismatches between the primers of SARS-CoV-2 and those of its closest relative SARS-CoV were below the nine variation tolerance threshold for RPA, the seven base-pair mismatches in crRNA regions could increase the specificity of the assay. We assayed the bulk RPA-Cas12a reaction targeting plasmids encoding the complete N gene from SARS-CoV-2, SARS-CoV, and MERS-CoV (FIGS. 11B and 11C). Positive fluorescence signals were observed with the SARS-CoV-2 plasmid but not the SARS-CoV or MERS-CoV plasmid (FIGS. 11B and 11C). The absence of cross-reactivity with other related coronaviruses tested validates the specificity of RADICA for SARS-CoV-2.

### Example 7

### Background Human DNA Tolerance Analysis of RADICA (Digital RPA-Cas12a)

Previous studies have reported that RPA reactions could be inhibited by high concentrations of background human DNA 33, 34. We therefore first tested the RPA-Cas12a bulk reaction under varying concentrations of background human DNA.

### Methods:

The RADICA reaction mix for SARS-CoV-2 DNA was prepared by mixing 300 nM forward primer (N-AIOD-F), 300 nM reverse primer (N-AIOD-R), 500 nM FQ reporter, 1×RPA rehydration buffer containing 1×RPA Pellet (TwistDx), 200 nM EnGen® Lba Cas12a (New England Biolabs), 200 nM crRNA (synthesized by N-AIOD-crRNA-IVT-F and T7-3G IVT primer), 1× Clarity™ JN solution (JN Medsys), followed by adding various amounts of DNA input with or without human genomic DNA, and 14 mM magnesium acetate. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### Results:

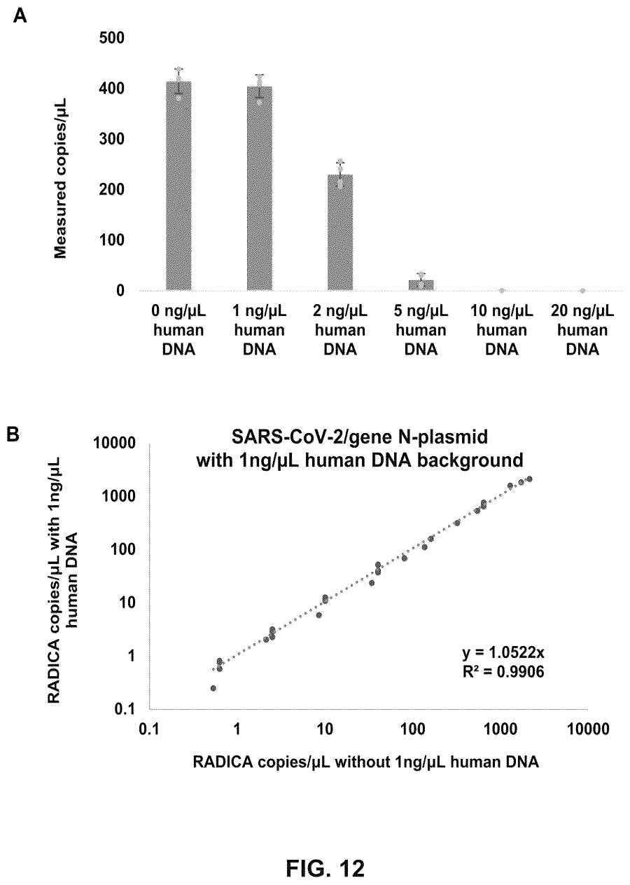

RPA reactions can be inhibited by high concentrations of background human DNA [Rohrman, B. and Richards-Kortum, R. Analytical chemistry 87: 1963-1967 (2015)]. We tested for possible inhibitory effects of background DNA on reactions carried out in small partitions. In an RPA-Cas12a reaction with 400 copies/μL of target DNA, 1 ng/μL of background human DNA (4350 human cells per reaction) did not affect the RADICA reaction (FIG. 12A). We observed inhibition of the reaction containing 2 ng/μL of background human DNA, and complete inhibition of the reaction containing >5 ng/μL of background human DNA (FIG. 12A). Since input DNA concentration used for RADICA-based detection is typically below 1 ng/μL, our findings support that background DNA does not inhibit the RADICA reaction of samples within the dynamic range to be used for testing.

We tested the effect of 1 ng/μL of background human DNA on RADICA reactions with various concentrations of target DNA (FIG. 12B). 1 ng/μL of background DNA did not affect reactions containing target DNA concentrations within the dynamic range of dPCR detection, i.e., 0.6 to 2027 copies/μL (FIG. 12B). Our findings confirm that background human DNA in the sample does not affect the absolute quantification of nucleic acids by RADICA.

### Example 8

### Quantitative Detection of SARS-CoV-2 RNA Using RADICA (Digital RPA-Cas12a)

As SARS-CoV-2 is an RNA virus, we tested whether RADICA could be combined with reverse transcription (RT) in a one-pot reaction for the absolute quantification of RNA. The method was optimized with the primers and crRNA shown in Table 3 for SARS-CoV-2 RNA detection.

TABLE 3
Primers and crRNA used in SARS-CoV-2 RNA detection

| Name | Sequence | Application |
|---|---|---|
| ssDNA-FQ reporter | /56-FAM/TTATT/3IABKFQ/ | Cas12a FQ |
| | | reporter |
| N-AIOD-F | AGGCAGCAGTAGGGGAACTTCTC | RADICA Primer |
| SEQ ID NO: 9 | CTGCTAGAAT | for N gene, N1 |
| | | region* |
| N-AIOD-R | TTGGCCTTTACCAGACATTTTGCT | RADICA Primer |
| SEQ ID NO: 10 | CTCAAGCTG | for N gene, N1 |
| | | region* |
| N-RPA-RR | AGCAGATTTCTTAGTGACAGTTTG | CRISPR Primer |
| SEQ ID NO: 13 | GCCTTGTTG | for N gene |
| N-AIOD-crRNA-IVT-F | ggctggcaatggcggtgatgATCTACACT | crRNA template |
| SEQ ID NO: 11 | TAGTAGAAATTACCCTATAGTGAG | for N gene, pair |
| | TCGTATTAATTTC | with T7-3G IVT |
| | | primer, also used |
| | | as crRNA-ssDNA1 |
| | | template |
| T7-3G IVT primer | GAAATTAATACGACTCACTATAGG | Universal primer |
| SEQ ID NO: 12 | G | used for crRNA |
| | | synthesis |
| T7-Cas12scaffold-F | gaaatTAATACGACTCACTATAGGT | Universal primer |
| SEQ ID NO: 14 | AATTTCTACTAAGTGTAGAT | used for crRNA |
| | | synthesis |
| NF-COV-F | CAACTTCCTCAAGGAACAACATT | RADICA Primer |
| SEQ ID NO: 15 | GCCAAAAGG | for N gene, NO |
| | | region |
| NF-COV-R | TGGAGTTGAATTTCTTGAACTGTT | RADICA Primer |
| SEQ ID NO: 16 | GCGACTAC | for N gene, NO |
| | | region |
| NF-COV-RR | GAGAAGTTCCCCTACTGCTG | RADICA Primer |
| SEQ ID NO: 17 | | for N gene, NO |
| | | region |
| NF-crRNA-1F | CTTCTACGCAGAAGGGAGCAatcta | crRNA template |
| SEQ ID NO: 18 | cacttagtagaaatta | for N gene, NO |
| | | region, pair with |
| | | T7-Cas12scaffold- |
| | | F |

*Ding, X. et al., Nature Communications 11:4711 (2020).

### Methods:

The RADICA reaction mix for SARS-CoV-2 DNA was prepared by mixing 300 nM forward primer (N-AIOD-F for N1 or NF-CoV-F for N0 region), 300 nM reverse primer (N-AIOD-R for N1 or NF-CoV-R for N0 region), 300 nM reverse primer2 (N-RPA-RR for N1 or NF-CoV-RR for N0 region), 500 nM FQ reporter, 1×RPA rehydration buffer containing 1× RPA Pellet (TwistDx), 200 nM EnGen® Lba Cas12a (New England Biolabs), 200 nM crRNA (synthesized by N-AIOD-crRNA-IVT-F and T7-3G IVT for N1; synthesized by NF-crRNA-1F and T7-Cas12scaffold-F for N0), 10 U/μL PhotoScript Reverse transcriptase (New England Biolabs) or 10 U/μL SuperScript™ IV Reverse Transcriptase (Invitrogen) and 0.5 U/μL RNase H (Invitrogen or New England Biolabs), 1× Clarity™ JN solution (JN Medsys), followed by adding various amounts of RNA target, and 14 mM magnesium acetate. All reactions were prepared on ice and samples were loaded within one minute to prevent premature target amplification. 15 μL of the mixture was loaded on the chip by a Clarity™ autoloader for sample partitioning. The reaction partitions were sealed with the Clarity™ Sealing Enhancer and 230 μL Clarity™ Sealing Fluid. The partitioned reactions were incubated in water baths or heat blocks at 42° C. for 1 hour, unless otherwise indicated. After incubation, a Clarity™ Reader was used to read the fluorescent signal in the partitions, and Clarity™ software was used to calculate input DNA copy numbers.

### Results:

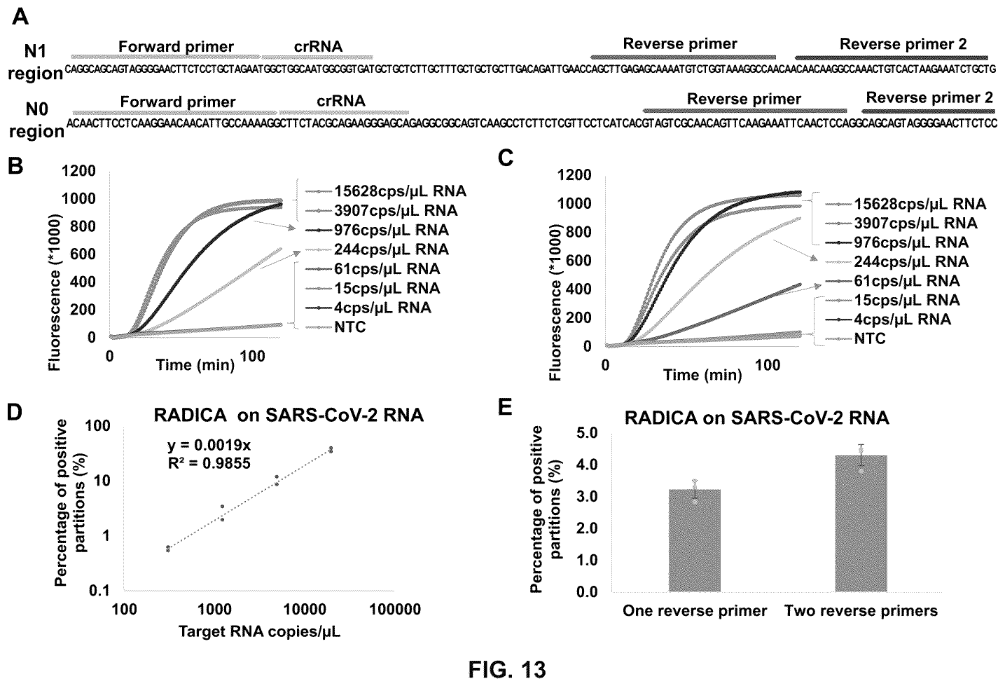

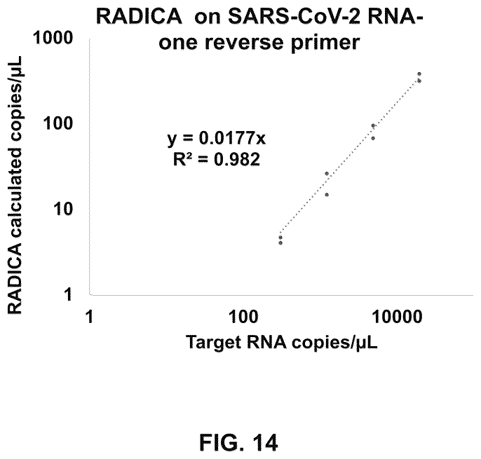

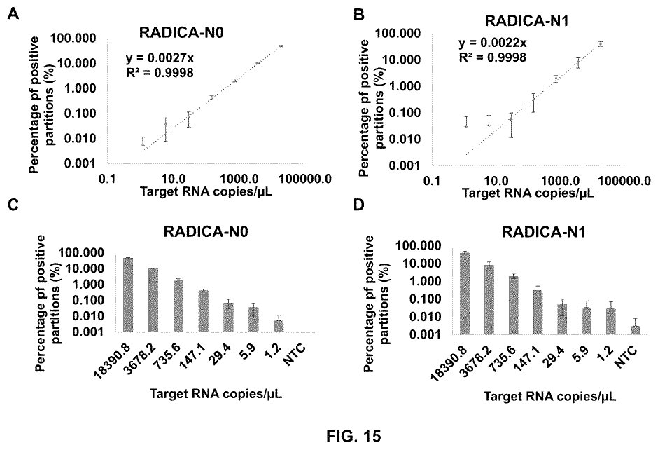

The sensitivity of the one-pot RT-RPA-Cas12a bulk reaction was lower-than-expected, with an LoD at 244 copies/μL, with an increased sensitivity (61 copies/μL) when two reverse primers were used (FIG. 13A-C). We then digitalized the one-pot RT-RPA-Cas12a reaction using digital chips and tested the results with various concentrations of RNA. Notably, we can see a good linear correlation between the target RNA concentration and the percentage of positive partitions (FIG. 13D). When Poisson distribution was used to calculate the copy number of RNA, 1 copy of input RNA resulted in an increase of only copies as calculated by RADICA, likely due to “molecular dropout” or low filling rate which was also observed in previous studies [Whale, A. S. et al. PloS one 8: e58177 (2013)](FIG. 14). We found that using two reverse primers instead of one reverse primer could increase the positive partition ratio for the same concentration of target RNA (FIG. 13E). Using the two-reverse-primer strategy, we designed two sets of primers/crRNA targeting different regions of N gene (N0 region: 478-620 bp, N1 region: 597-754 bp, FIG. 13A) and tested the behaviour of RADICA on serial dilutions of SARS-CoV-2 RNA in the background of 1 ng/μL human genomic DNA. Excellent linear relationships were observed between the RNA copy number and the positive partition ratio in both the two primers/crRNA sets (FIGS. 15A and 15B). Using both primer/crRNA sets, 1.2 copies/μL of RNA could be detected on the digital chip, which is much more sensitive than the bulk reaction (FIGS. 15C and 15D). These results support that RADICA can quantitatively detect RNA directly with better sensitivity than bulk reactions.

### Example 9

Absolute Quantification of Epstein-Barr Virus from Infected B Cells by RADICA (Digital RPA-Cas12a)

We tested the ability of RADICA to perform absolute quantification on Epstein-Barr virus (EBV), a member of the human herpesvirus (HHV4) reported as viral contamination in the biologic and cell manufacturing process [Barone, P. W. et al. Nature biotechnology 38: 563-572 (2020)]. The primers and crRNA shown in Table 4 were used for Epstein-Barr virus detection in infected B cells.

TABLE 4
Primers and crRNA used in EBV detection

| Name | Sequence | Application |
|---|---|---|
| ssDNA-FQ reporter | /56-FAM/TTATT/3IABKFQ/ | Cas12a FQ |
| | | reporter |
| T7-Cas12scaffold-F | gaaatTAATACGACTCACTATAGGT | Universal primer |
| SEQ ID NO: 14 | AATTTCTACTAAGTGTAGAT | used for crRNA |
| | | synthesis |
| EBNA-PCR-F | TCATCATCATCCGGGTCTCC | qPCR and dPCR |
| SEQ ID NO: 19 | | primer for EBV |
| | | clinical sample^{∧} |
| EBNA-PCR-R | GCTCACCATCTGGGCCAC | qPCR and dPCR |
| SEQ ID NO: 20 | | primer for EBV |
| | | clinical sample^{∧} |
| EBNA-P-FAM | /56-FAM/CC TCC AGG T/ZEN/A | qPCR and dPCR |
| SEQ ID NO: 21 | GAA GGC CAT TTT TCC ACC CTG | probe for EBV |
| | TAG/3IABKFQ/ | clinical sample^{∧} |
| BamHIW-PCR-44F | CCCAACACTCCACCACACC | qPCR and dPCR |
| SEQ ID NO: 22 | | primer for EBV |
| | | clinical sample# |
| BamHIW-PCR-119R | TCTTAGGAGCTGTCCGAGGG | qPCR and dPCR |
| SEQ ID NO: 23 | | primer for EBV |
| | | clinical sample# |
| BamHIW-P-HEX | /5HEX/CA CAC ACT A/ZEN/C ACA | qPCR and dPCR |
| SEQ ID NO: 24 | CAC CCA CCC GTC TC/3IABKFQ/ | probe for EBV |
| | | clinical sample# |
| EBV-EBNA1-F2 | GCCGGTGTGTTCGTATATGGAGG | RADICA forward |
| SEQ ID NO: 25 | TAGTAAGAC | primer for EBV |
| | | EBNA-1 |
| EBV-EBNA1-R2 | ATTCCAAAGGGGAGACGACTCAA | RADICA reverse |
| SEQ ID NO: 26 | TGGTGTAA | primer for EBV |
| | | EBNA-1 |
| EBNA-2R1-crRNAR | ACGACATTGTGGAAYAGCAAGGat | crRNA template |
| SEQ ID NO: 27 | ctacacttagtagaaatta | for EBV EBNA-1, |
| | | pair with T7- |
| | | Cas12scaffold-F |
| EBV-BamHIW-F3 | CTGCCCCTGGTATAAAGTGGTCC | RADICA forward |
| SEQ ID NO: 28 | TGCAGCTATT | primer for EBV |
| | | BamHI-W |
| EBV-BamHIW-R3 | GGCTAGGGAGAGGTAGAAGACC | RADICA reverse |
| SEQ ID NO: 29 | CCCTCTTACA | primer for EBV |
| | | BamHI-W |
| BamHIW-3F-crRNAR | TCTGGTCGCATCAGAGCGCCatcta | crRNA template |
| SEQ ID NO: 30 | cacttagtagaaatta | for EBV BamHI-W, |
| | | pair with T7- |
| | | Cas12scaffold-F |

^{∧}Vo, J. H. et al., Scientific Reports 6:13-13 (2016).
#Tay, J. K. et al., International Journal of Cancer 16L2923-2931 (2020).

### Methods:

Primer and crRNA Design for RADICA Targeting EBV:

Epstein-Barr virus primers and crRNA were designed based on consensus sequences of 16 virus genomes, including both type I and type II EBV (NCBI: AP015016.1, AY961628.3, HQ020558.1, JQ009376.2, KC207813.1, KC207814.1, KC440851.1, KC440852.1, KC617875.1, KF373730.1, KF717093.1, KP735248.1, LN827800.1, NC_007605.1, NC_009334.1, V01555.2).

### EBV Quantification by Digital PCR:

Serial dilutions of EBV DNA were used for dPCR quantification by Clarity™ Epstein-Barr Virus Quantification Kit (JN Medsys) or primers and probes from published papers [Tay, J. K. et al. International Journal of Cancer 146: 2923-2931 (2020); Vo, J. H. et al. Scientific reports 6: 13-13 (2016)] with TaqMan™ Fast Advanced Master Mix (Applied Biosystems), 1× Clarity™ JN solution (JN Medsys) according to the manufacturer's protocol.

Growing EBV-2 from Jijoye Cells:

Jijoye cells were treated with 4 mM sodium butyrate and 24 ng/ml tetradecanoyl phorbol acetate (TPA). Supernatants were harvested 4-5 days post-treatment by centrifugation at 4,000 g for 20 min and passing over a 0.45 μm filter to remove cellular debris. Viral particles were pelleted by ultracentrifugation at 20,000 rpm for 90 min and resuspended in 1/100 the initial volume using complete RPMI or PBS if viruses were to be further purified. Concentrated viruses were further purified using OptiPrep gradient density ultracentrifugation at 20,000 rpm for 120 min, and the virus interface band was collected and stored at −80° C. for downstream analysis.

Epstein-Barr Virus DNA Extraction from Jijoye Cells:

EBV DNA was extracted using the QIAamp DNA Mini Kit (QIAGEN) according to the manufacturer's protocol.

### RADICA Quantification of Epstein-Barr Virus DNA:

Each 15 μL RADICA reaction consisted of 300 nM forward primer (EBV-EBNA1-F2 or EBV-BamHIW-F3), 300 nM reverse primer (EBV-EBNA1-R2 or EBV-BamHIW-R3), 500 nM FQ reporter, 1×RPA rehydration buffer containing 1×RPA Pellet (TwistDx), 200 nM EnGen® Lba Cas12a (New England Biolabs), 200 nM EBNA-2R1-crRNA or BamHIW-3F-crRNA, 0.01 mg/mL BSA, 1× Clarity JN Solution (JN Medsys), different concentrations of DNA or controls, and 14 mM magnesium acetate. Each reaction mix was partitioned in the Clarity Digital PCR tube-strip (JN Medsys) followed by 42° C. incubation for 1 h in a water bath. After incubation, a Clarity™ Reader was used to read the fluorescent signal in the partitions, and Clarity™ software was used to calculate input DNA copy numbers.

### Results:

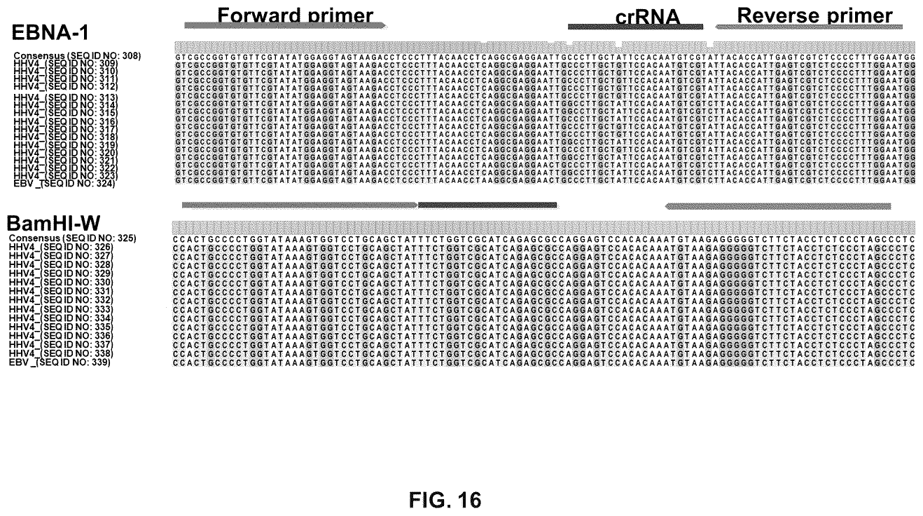

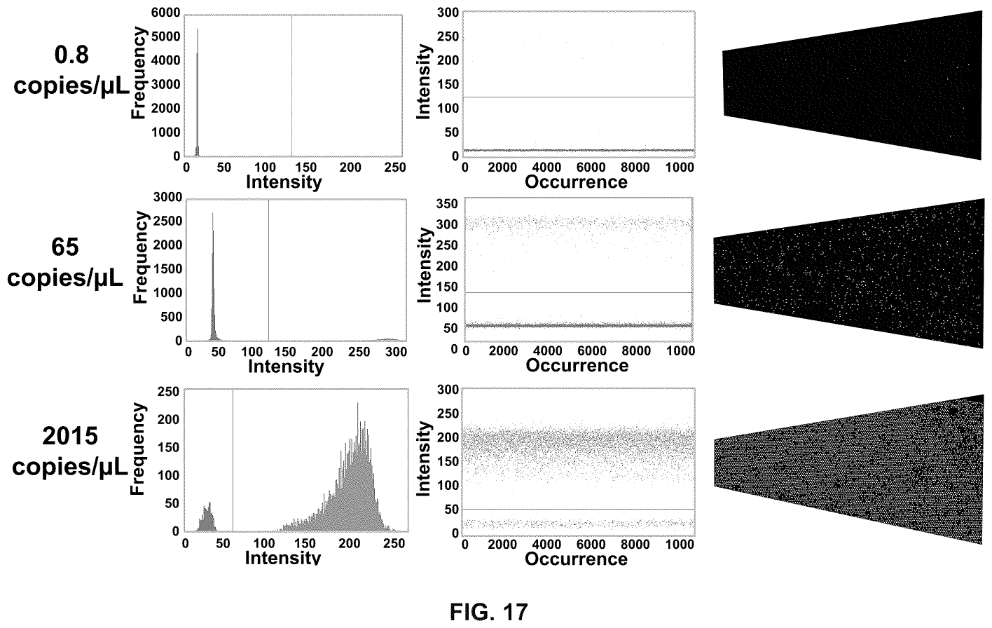

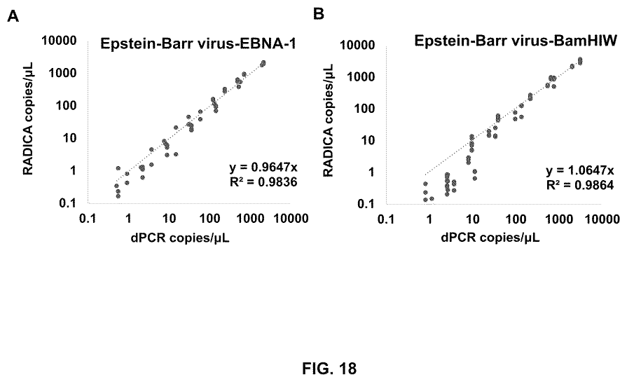

To design primers and crRNA that were universal to both type I and type II EBV, we analyzed the genomes of 16 EBV strains and identified the conserved regions across all 16 strains. A conserved DNA region within the Epstein-Barr nuclear antigen 1 (EBNA1) and repetitive BamHI-W sequences were used as the target sequences (FIG. 16). Viral DNA extracted from chemically-induced EBV-harboring human B cells, diluted to concentrations ranging from 0.5 to 2100 copies/μL, was used as the target DNA in both RADICA and dPCR reactions. For RADICA-based detection, samples loaded in the partition chip were incubated for 1 h at 42° C., followed by endpoint fluorescence detection and copy number determination. Notably, the positive partition signal increased with an increase in the concentration of input EBV DNA (FIG. 17). The copy numbers measured by RADICA are in full concordance (R² value>0.98) with those measured by dPCR (FIGS. 18A and 18B). Our findings validate the accuracy and sensitivity of RADICA for the absolute quantification of viral DNA within an hour in human samples, a four-fold reduction in reaction time compared to dPCR-based detection.

### Example 10

Clinical Validation of RADICA (Digital RPA-Cas12a) and Comparison with qPCR and dPCR

To validate RADICA in clinical samples, we compared RADICA with qPCR- and dPCR-based quantification methods to analyze the EBV load in 79 serum samples obtained from 39 nasopharyngeal cancer (NPC) patients and 40 healthy controls. NPC is an EBV-associated malignancy and the circulating EBV cell-free DNA is elevated in 53-96% of NPC patients [Tay, J. K. et al. International Journal of Cancer 146: 2923-2931 (2020)].

### Methods:

### Clinical Samples for Epstein-Barr Virus Detection:

Two sets of clinical samples from a serum bank of nasopharyngeal cancer patients and healthy controls were used in this study. The first set comprised 79 serum samples of which 39 were from nasopharyngeal cancer (NPC) patients and 40 were from healthy controls. The second set comprised of 66 serum samples taken from 22 NPC patients at three time points: at the time of initial diagnosis, one year after treatment and at the time of recurrence. All participants were recruited with informed consent, and the study was approved by the Institutional Review Board of the National Healthcare Group, Singapore (Approval numbers: 2006/00149, 2006/00409).

Epstein-Barr Virus DNA Extraction from Serum:

EBV cell-free DNA was extracted from 200 μL of serum using the ReliaPrep™ Blood gDNA Miniprep System (Promega) according to the manufacturer's protocol. The DNA was eluted using 50 μL of ddH₂O and further diluted using another 50 μL of ddH₂O, resulting in 100 μL of DNA solution.

qPCR Quantification of Epstein-Barr Virus in Serum Cell-Free DNA:

qPCR was performed using 400 nM EBNA-PCR-F, 400 nM EBNA-PCR-R, 200 nM EBNA-P-FAM, 200 nM BamHIW-PCR-44F, 200 nM BamHIW-PCR-119R, 100 nM BamHIW-P-HEX, 3 μL of serum DNA (after 1:1 dilution) or controls in 1× TaqMann™ Fast Advanced Master Mix (Applied Biosystems). Each reaction mix was incubated at 50° C. 2 min to allow UNG to degrade carry-over PCR products, followed by 1 cycle at 95° C. for 2 min, 40 cycles at 95° C. for 3 s and 59° C. for 30 s. A standard curve using DNA extracted from the Jijoye EBV positive cell line was run in parallel with each reaction to quantify the input DNA concentration.

dPCR Quantification of Epstein-Barr Virus in Serum Cell-Free DNA:

dPCR was performed using the Clarity Digital PCR System (JN Medsys). Each 15 μL dPCR reaction consisted of 400 nM EBNA-PCR-F, 400 nM EBNA-PCR-R, 200 nM EBNA-P-FAM, 200 nM BamHIW-PCR-44F, 200 nM BamHIW-PCR-119R, 100 nM BamHIW-P-HEX, 1× Clarity JN Solution (JN Medsys), 1× TaqMann™ Fast Advanced Master Mix (Applied Biosystems), and 3 μL of serum DNA or controls. Each reaction mix was partitioned in the Clarity Digital PCR tube-strip (JN Medsys) followed by 1 cycle at 95° C. for 10 min, 40 cycles at 95° C. for 50 s and 57° C. for 90 s and 1 cycle at 70° C. for 5 min. After thermal cycling, a Clarity™ Reader was used to read the fluorescent signal in the partitions, and Clarity™ software was used to calculate input DNA copy numbers.

### RADICA Quantification of Epstein-Barr Virus in Serum Cell-Free DNA:

Each 15 μL RADICA reaction consisted of 300 nM EBV-BamHIW-F3, 300 nM EBV-BamHIW-R3, 500 nM FQ reporter, 1×RPA rehydration buffer containing 1×RPA Pellet (TwistDx), 200 nM EnGen® Lba Cas12a (New England Biolabs), 200 nM BamHIW-3F-crRNA, 0.01 mg/mL BSA, 1× Clarity JN Solution (JN Medsys), 3 μL of serum DNA or controls, and 14 mM magnesium acetate. Each reaction mix was partitioned in the Clarity Digital PCR tube-strip (JN Medsys) followed by 42° C. incubation for 1 h in a water bath. After incubation, a Clarity™ Reader was used to read the fluorescent signal in the partitions, and Clarity™ software was used to calculate input DNA copy numbers.

### Results:

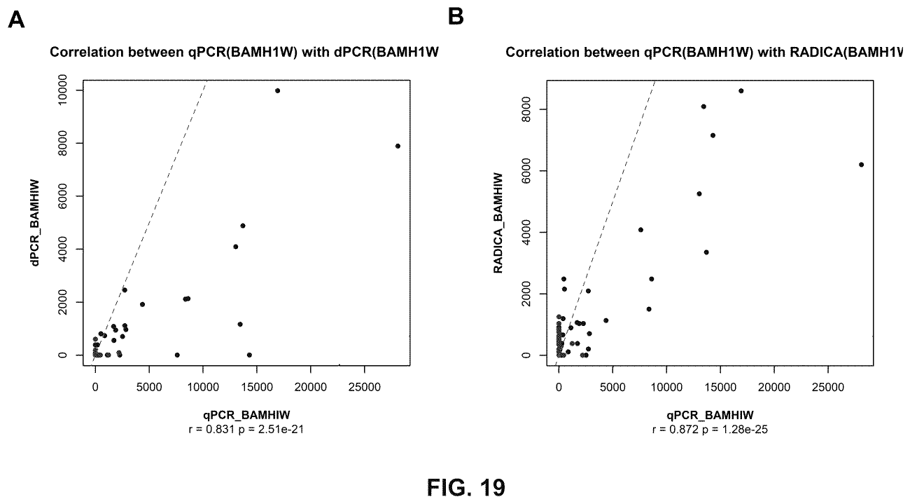

Cell-free DNA from 79 frozen serum samples were blinded and the viral load was quantified using the EBV BamHI-W target. First, to confirm the integrity of the frozen serum samples after long-term storage, qPCR-based quantification of the EBV load in each frozen serum sample was performed and found to be comparable with that obtained previously using the fresh serum. We next compared dPCR- and RADICA-based EBV quantification with that obtained from qPCR on the same serum samples, and found a high correlation between both methods and qPCR, with RADICA demonstrating a higher correlation with qPCR (r=0.872, p=1.28e-²⁵) compared to dPCR (r=0.831, p=2.51e-²¹) (FIG. 19). These results suggest the superior performance of RADICA over dPCR and the ability of RADICA to match the clinically used qPCR-based detection of EBV in serum samples.

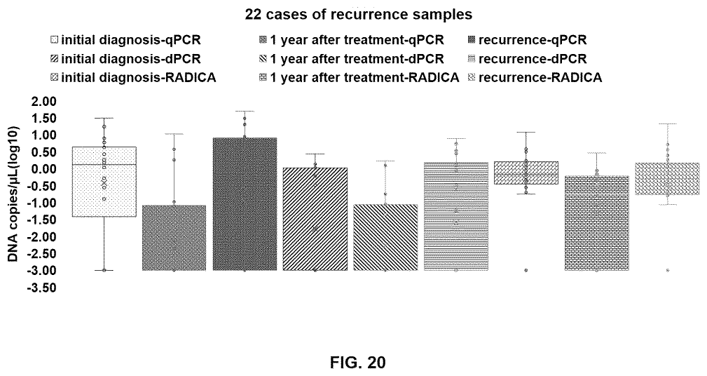

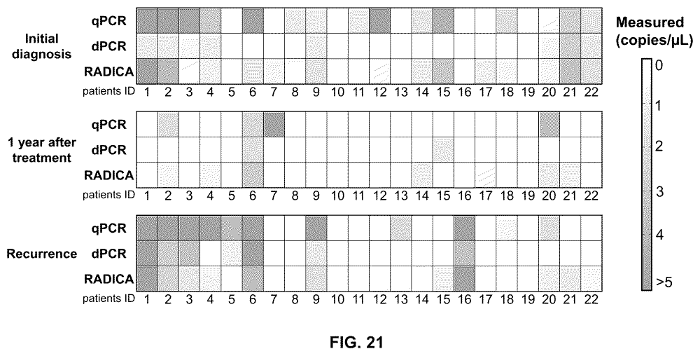

As EBV cell-free DNA is routinely used to monitor the virus load in NPC patients after treatment, frozen serum samples obtained from 22 NPC patients at their initial diagnoses, one year after treatment and the point of recurrence were blinded, and EBV loads were quantified by qPCR, dPCR and RADICA (FIGS. 20 and 21). Similar to qPCR and dPCR, RADICA-based EBV quantification on the 22 NPC patients showed that the viral load in the serum decreased after treatment and increased at the time of recurrence, suggesting that RADICA's ability to absolutely quantify nucleic acids can be used to monitor and compare viral load over time in NPC patients' following treatment (FIG. 20). When analyzing the DNA copy number in each of the patients, we noted that the low EBV load in serum cell-free DNA in NPC samples (most of them ˜1 copy/μL, equals to 12 copies per reaction), as well as the fact that EBV can be detected in healthy individuals further challenged the sensitivity and quantification ability of these three methods. Despite the low copy number nature of the cell-free DNA samples, RADICA is still in higher concordance with qPCR results than dPCR by detecting EBV DNA in patients 6 and 15 at initial diagnosis and patient 4 at the time of recurrence while dPCR cannot (FIG. 21). Together, these results demonstrate the ability of RADICA in the absolute quantification of viral load in clinical samples.

### Example 11

Human genomic DNA is usually used as a control target to indicate the success of the reaction. Thus, we designed primers and crRNAs targeting human RNAse P gene and primer, and screened these primers and crRNAs (Table 5) using bulk RPA-Cas12a assay.

TABLE 5
Primers and crRNA used in human detection

| Name | Sequence | Application |
|---|---|---|
| ssDNA-FQ reporter | /56-FAM/TTATT/3IABKFQ/ | Cas12a FQ |
| | | reporter |
| T7-Cas12scaffold-F | gaaatTAATACGACTCACTATAGGT | Universal primer |
| SEQ ID NO: 14 | AATTTCTACTAAGTGTAGAT | used for crRNA |
| | | synthesis |
| RPP30-4-F | aaattacatctggtctcttccttcactgcttca | RPA-RNAseP |
| SEQ ID NO: 31 | | primer |
| RPP30-4-R | taaattatttccaaagttggttcagtccgatgc | RPA-RNAseP |
| SEQ ID NO: 32 | | primer |
| RPP30-5-F | gccagatgtttgaatattttaagagcttctttcg | RPA-RNAseP |
| SEQ ID NO: 33 | | primer |
| RPP30-5-R | tgaagcagtgaaggaagagaccagatgtaattt | RPA-RNAseP |
| SEQ ID NO: 34 | | primer |
| RNAseP-RPA-5L | tgttttaagcttctttcatgtattcaaatcagca | RPA-RNAseP |
| SEQ ID NO: 35 | | primer |
| RNAseP-RPA-5R | catgtgtatcctctctccttccacaaattctat | RPA-RNAseP |
| SEQ ID NO: 36 | | primer |
| RNAseP-RPA-6L | tgcaatattaatgtaagggctctaaaacaatgg | RPA-RNAseP |
| SEQ ID NO: 37 | | primer |
| RNAseP-RPA-6R | aaaaattgtattttctccaacccgcagaacagt | RPA-RNAseP |
| SEQ ID NO: 38 | | primer |
| 30-CR4-F | tgcctacgtaaggtctttga | RNAseP Cas12a |
| SEQ ID NO: 39 | atctacacttagtagaaatta | crRNA template |
| 30-CR4-R | tttgctatttttaatacagc | RNAseP Cas12a |
| SEQ ID NO: 40 | atctacacttagtagaaatta | crRNA template |
| 30-CR5-F | aaagtttcttgttcatactc | RNAseP Cas12a |
| SEQ ID NO: 41 | atctacacttagtagaaatta | crRNA template |
| 30-CR5-R | tacatgtgtatcctctctcc | RNAseP Cas12a |
| SEQ ID NO: 42 | atctacacttagtagaaatta | crRNA template |
| P-CR5-F | tttttttctaagaaattgct | RNAseP Cas12a |
| SEQ ID NO: 43 | atctacacttagtagaaatta | crRNA template |
| P-CR5-R | agcaatttcttagaaaaaaa | RNAseP Cas12a |
| SEQ ID NO: 44 | atctacacttagtagaaatta | crRNA template |
| P-CR6-F | agtagagccagaggtataac | RNAseP Cas12a |
| SEQ ID NO: 45 | atctacacttagtagaaatta | crRNA template |
| P-CR6-R | tgtctttctcttgcttaaaa | RNAseP Cas12a |
| SEQ ID NO: 46 | atctacacttagtagaaatta | crRNA template |

### Methods:

Screen the primer and crRNA using RPA-Cas12a bulk assay: The one-pot reaction combining RPA-DNA amplification and Cas12a detection was performed as follows: 300 nM forward primer, 300 nM reverse primer, 500 nM FQ reporter, 1×RPA rehydration buffer containing 1×RPA Pellet (TwistDx), 200 nM EnGen® Lba Cas12a (New England Biolabs), 200 nM crRNA, were prepared followed by adding various amounts of DNA input, and 14 mM magnesium acetate. The reaction mixture was incubated at 42° C. unless otherwise indicated and fluorescence kinetics were monitored every 1 min.

RADICA quantification of human DNA: Each 15 μL RADICA reaction consisted of 300 nM forward primer (RNAseP-RPA-6L), 300 nM reverse primer (RNAseP-RPA-6R), 500 nM FQ reporter, 1×RPA rehydration buffer containing 1×RPA Pellet (TwistDx), 200 nM EnGen® Lba Cas12a (New England Biolabs), 200 nM P-CR6-F-crRNA, 0.01 mg/mL BSA, lx Clarity JN Solution (JN Medsys), different concentrations of DNA or controls, and 14 mM magnesium acetate. Each reaction mix was partitioned in the Clarity Digital PCR tube-strip (JN Medsys) followed by 42° C. incubation for 1 h in a water bath. After incubation, a Clarity™ Reader was used to read the fluorescent signal in the partitions, and Clarity™ software was used to calculate input DNA copy numbers.

### Results:

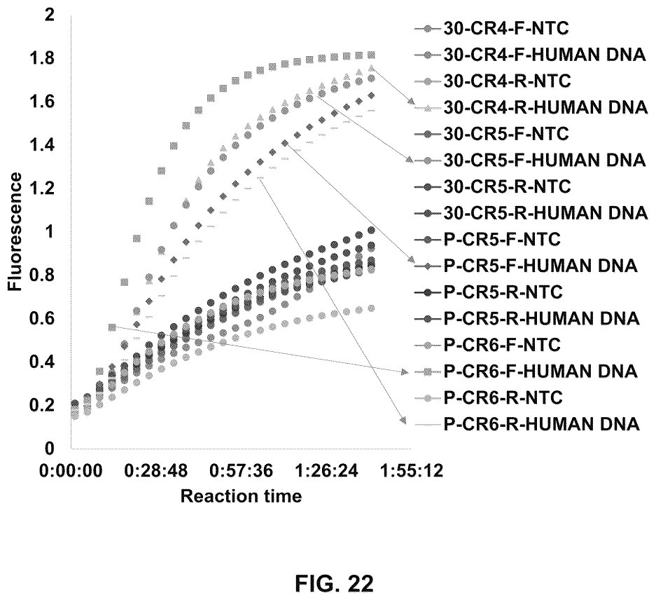

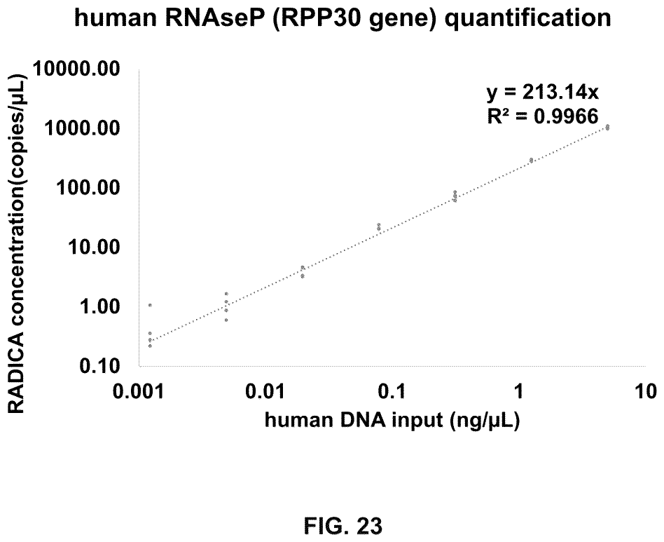

We designed primers and crRNAs targeting human RNAse P gene and primer, screened these primers and crRNAs using bulk RPA-Cas12a assay (FIG. 22), and crRNA set with the fast speed were used for RADICA. Human genomic DNA were serial diluted to different concentrations and tested using RADICA. For RADICA-based detection, samples loaded in the partition chip were incubated for 1 h at 42° C., followed by endpoint fluorescence detection and copy number determination. Based on the result, there is high linear relationship between the concentration of input human DNA and RADICA result (R²>0.99) (FIG. 23). The above results validate the detection and quantification ability of RADICA in detecting human genes.

### Example 12

### Further Example of RADICA Design: Digital RT-LAMP-Cas12b

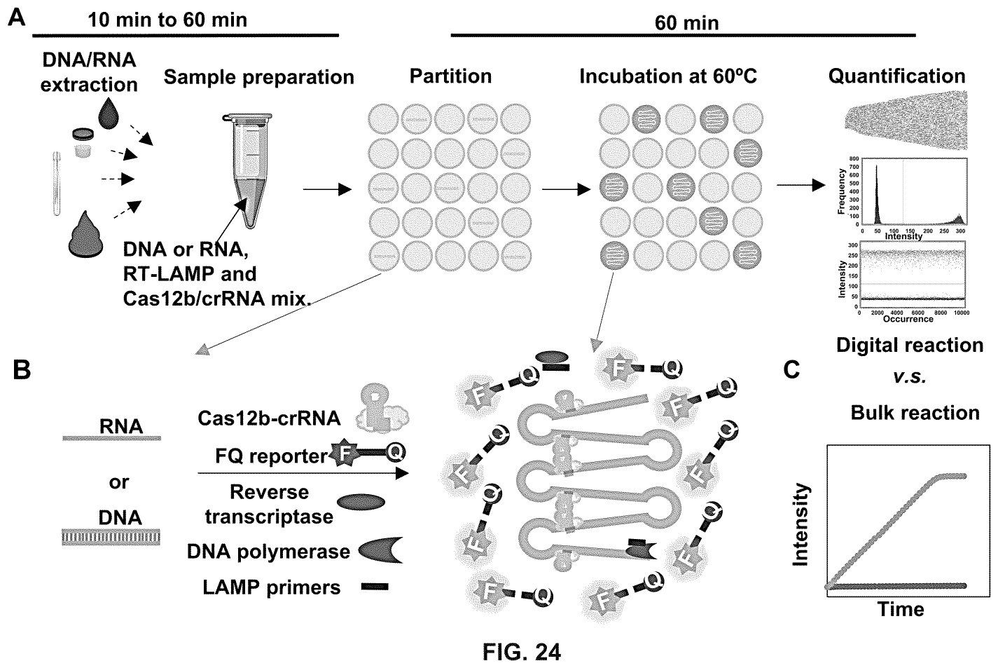

Another format of RADICA combines RT-LAMP and Cas12b in a one-pot reaction and digitalizes the reaction into thousands of nanoliter or sub-nanoliter reactions for quantitative results. The design principle of RADICA is illustrated in FIG. 24. First, the DNA or RNA is extracted from its source and assembled with WarmStart RTx reverse transcriptase (only needed for RNA sample), Bst 2.0 WarmStart DNA polymerase, LAMP primers, Cas12b/crRNA, and a quenched fluorescent (FQ) reporter. Then the reaction mixture is divided into thousands of partitions on a digital chip, followed by incubating in a 60° C. water bath. In each of the independent partitions containing the target, the RT-LAMP reaction starts to amplify the DNA/RNA exponentially. Simultaneously, the amplified DNA is identified by Cas12b/crRNA by sequence complementarity, which in turn triggers Cas12b, via its trans cleavage activity, to cut the FQ reporter and generate increased fluorescence (FIG. 24B). Thus, the partitions containing target DNA or RNA emit a positive fluorescent signal while the partitions without target DNA or RNA emit only the baseline signal (background).

As the RT-LAMP reaction is warm-start, the reaction will be inhibited at temperatures below 45° C. and will start only after the samples are partitioned and incubated at 60° C., which enables room-temperature reaction setup and increases the accuracy and consistency of the results. The Cas enzyme we used was the thermostable Cas12b from Alicyclobacillus acidiphilus (AapCas12b), which has been shown to be compatible with the one-pot RT-LAMP reaction with high sensitivity [Joung, J. et al. New England Journal of Medicine (2020)]. Compared to a bulk reaction (like the conventional SHERLOCK reaction) [Joung, J. et al. New England Journal of Medicine (2020)] (FIG. 24C), the digital reaction is quantified by assigning each positive and negative partition as “one” or “zero” and calculating the percentage of positive partitions. As the partitions are physically separated, the digital reaction eliminates interference among individual reaction wells.

### Example 13

### Optimization of the RT-LAMP-Cas12b Assay for RADICA (Digital RT-LAMP-Cas12b) Detection

Systematic studies of one-pot RT-LAMP-Cas12b reaction were conducted to optimize the assay performance. The primers and crRNAs used are shown in Table 6.

TABLE 6
Primers, crRNA for RADICA (digital RT-LAMP-Cas12b) optimization.

| Name | Sequence | Application |
|---|---|---|
| FQ5T | /56-FAM/TTTTT/3IABKFQ/ | FQ reporter |
| FQ8T | /56-FAM/TTTTTTTT/3IABKFQ/ | FQ reporter |
| FQ12T | /56-FAM/TTTTTTTTTTTT/3IABKFQ/ | FQ reporter |
| SEQ ID NO: | | |
| 47 | | |
| FQ16T | /56-FAM/TTTTTTTTTTTTTT/3IABKFQ/ | FQ reporter |
| SEQ ID NO: | | |
| 48 | | |
| FQ20T | /56- | FQ reporter |
| SEQ ID NO: | FAM/TTTTTTTTTTTTTTTTTTT/3IABKFQ/ | |
| 49 | | |
| FQ8A | /56-FAM/AAAAAAAA/3IABKFQ/ | FQ reporter |
| FQ8G | /56-FAM/GGGGGGGG/3IABKFQ/ | FQ reporter |
| FQ8C | /56-FAM/CCCCCCCC/3IABKFQ/ | FQ reporter |
| FQ8AT | /56-FAM/TTATTATT/3IABKFQ/ | FQ reporter |
| N2-LAMP- | GUCUAGAGGACAGAAUUUUUCAACGGG | SARS-CoV-2 |
| crRNA11 | UGUGCCAAUGGCCACUUUCCAGGUGG | crRNA for Cas12b* |
| SEQ ID NO: | CAAAGCCCGUUGAGCUUCUCAAAUCUG | |
| 50 | AGAAGUGGCACCGAAGAACGCUGAAGC | |
| | GCUG | |
| N2-WSLAMP- | GCTGCTGAGGCTTCTAAG | SARS-CoV-2 |
| F3 | | LAMP primers* |
| SEQ ID NO: | | |
| 51 | | |
| N2-WSLAMP- | GCGTCAATATGCTTATTCAGC | SARS-CoV-2 |
| B3 | | LAMP primers* |
| SEQ ID NO: | | |
| 52 | | |
| N2-WSLAMP- | GCGGCCAATGTTTGTAATCAGTAGACGT | SARS-CoV-2 |
| FIP | GGTCCAGAACAA | LAMP primers* |
| SEQ ID NO: | | |
| 53 | | |
| N2-WSLAMP- | TCAGCGTTCTTCGGAATGTCGCTGTGTA | SARS-CoV-2 |
| BIP | GGTCAACCACG | LAMP primers* |
| SEQ ID NO: | | |
| 54 | | |
| N2-WSLAMP- | CCTTGTCTGATTAGTTCCTGGT | SARS-CoV-2 |
| LoopF | | LAMP primers* |
| SEQ ID NO: | | |
| 55 | | |
| N2-WSLAMP- | TGGCATGGAAGTCACACC | SARS-CoV-2 |
| LoopB | | LAMP primers* |
| SEQ ID NO: | | |
| 56 | | |

*Joung, J. et al., New England Journal of Medicine (2020).

### Methods:

### Bulk RT-LAMP-Cas12b Reactions:

The DNA/RNA target samples were mixed with 1.6 μM FIP primers, 1.6 μM BIP primers, 0.2 μM F3 primers, 0.2 μM B3 primers, 0.4 μM LoopF primers, 0.4 μM LoopB primers, 1.4 mM dNTPs, 8 mM MgSO₄, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, and 50 nM crRNA in 1× isothermal amplification buffer, unless otherwise indicated. To detect the LAMP signal, 250 mM SYTO-82 fluorescent nucleic acid stain was added to the reaction. The reaction mixture was incubated at 60° C. and fluorescence kinetics were monitored for 1-2 h using Roche Light Cycler 96. The fluorescence values displayed in the results were the fluorescence levels determined by Roche Light Cycler 96 software. To mimic the complexity of real samples, 1 ng/μL human genomic DNA was also added to the reactions.

### RADICA Reactions by 01Acuity Digital Nanoplate:

The RADICA reaction was prepared by adding 250 nM Cyanine 680 succinimidyl ester (biotium) as reference dye to the RT-LAMP-Cas12b bulk reactions stated above and partitioned on QIAcuity digital nanoplates (˜0.91 nL partition volume, 26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

### Results:

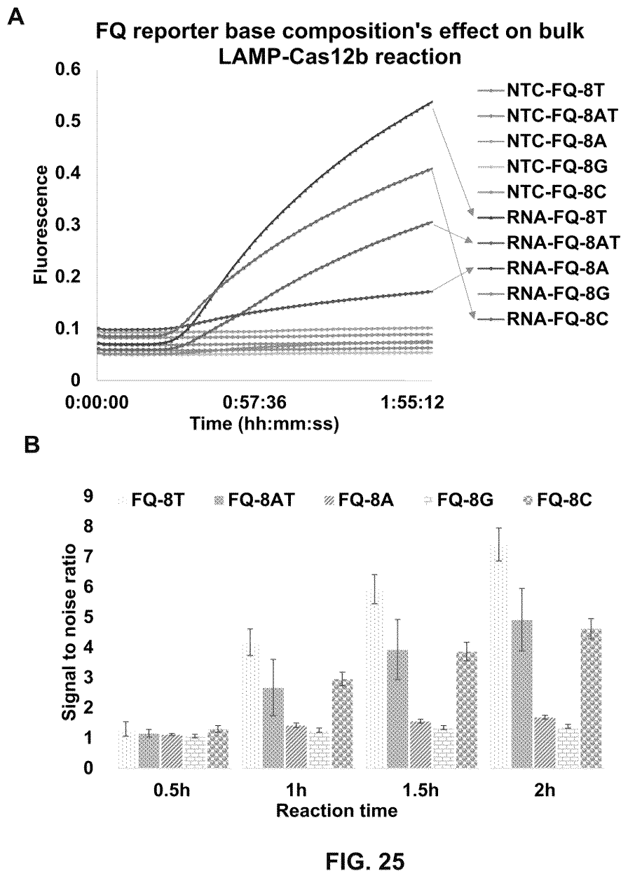

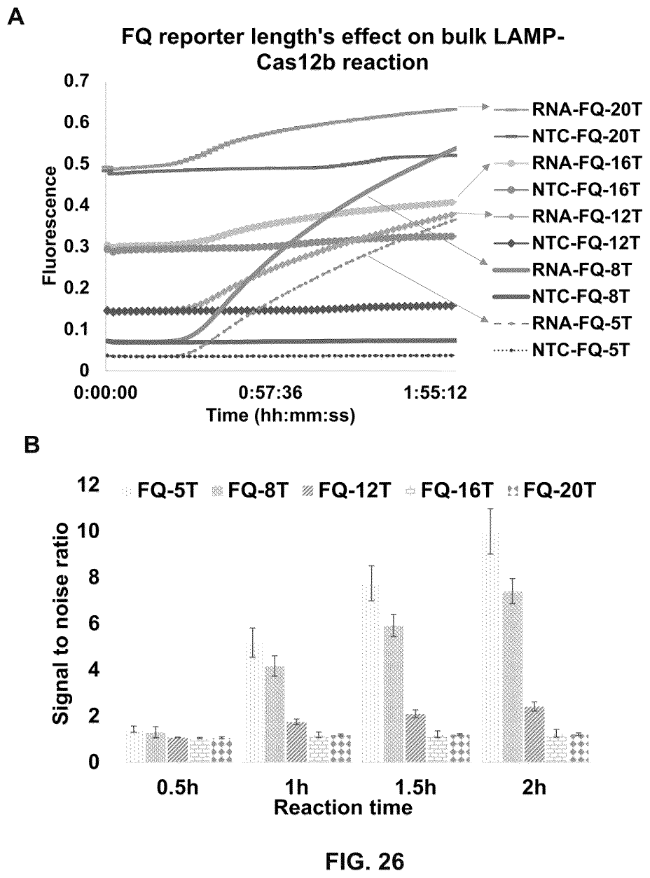

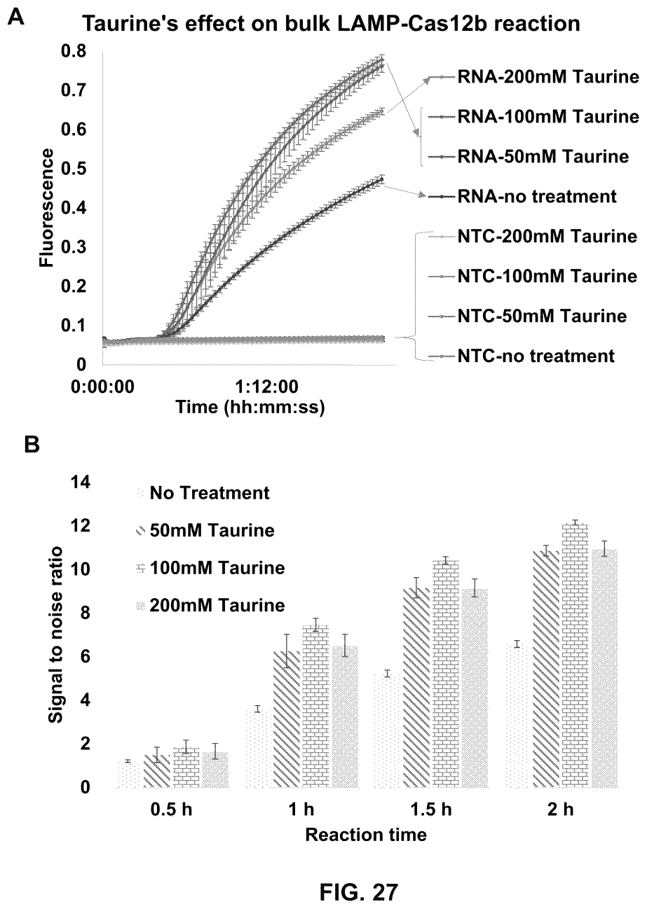

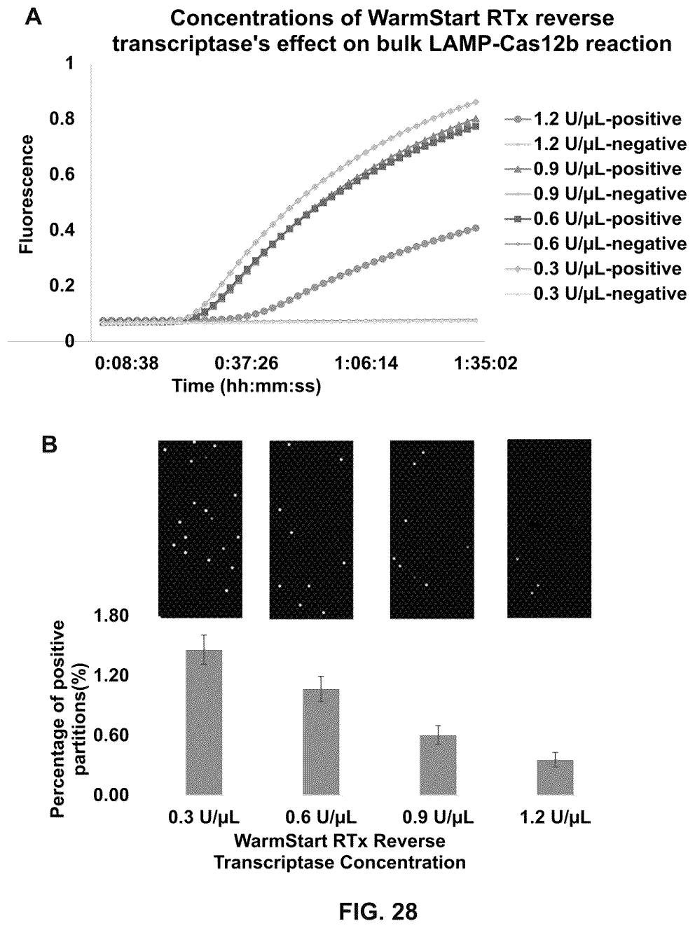

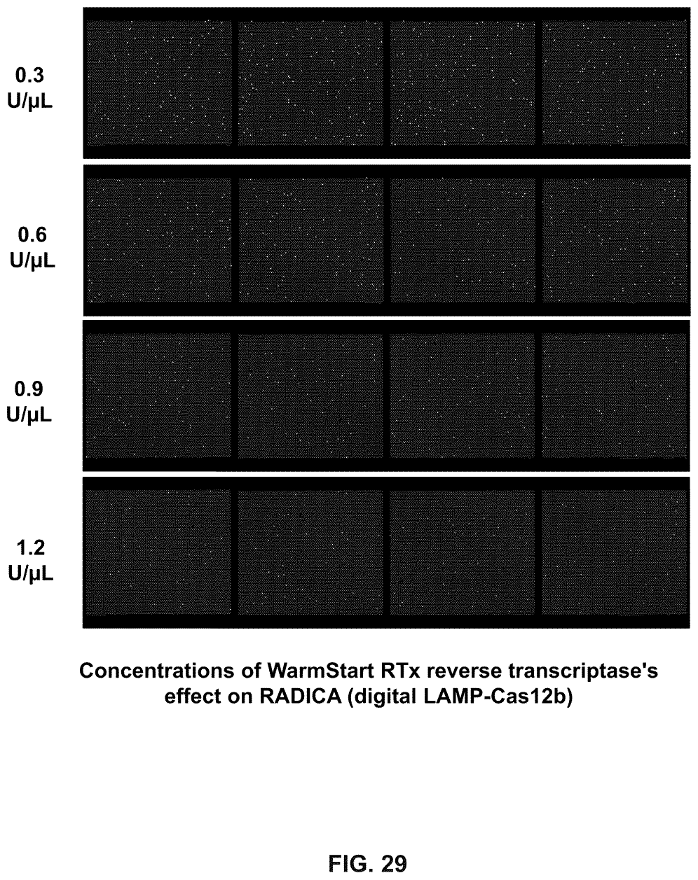

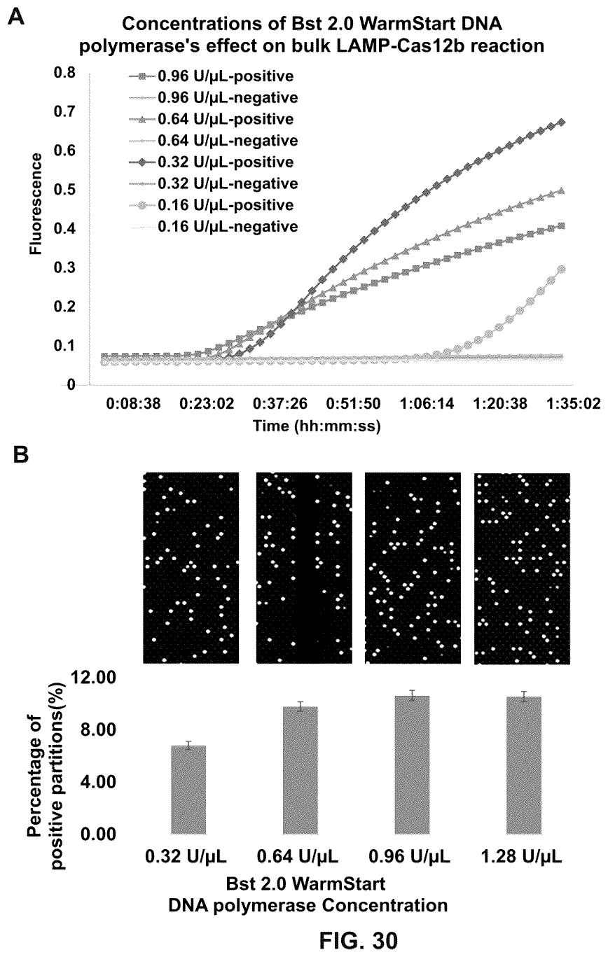

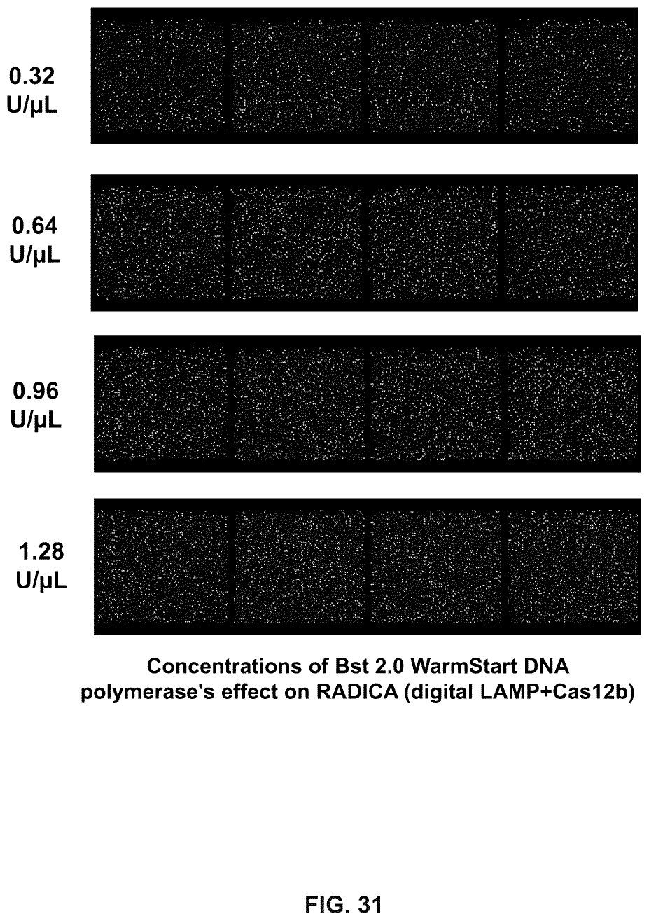

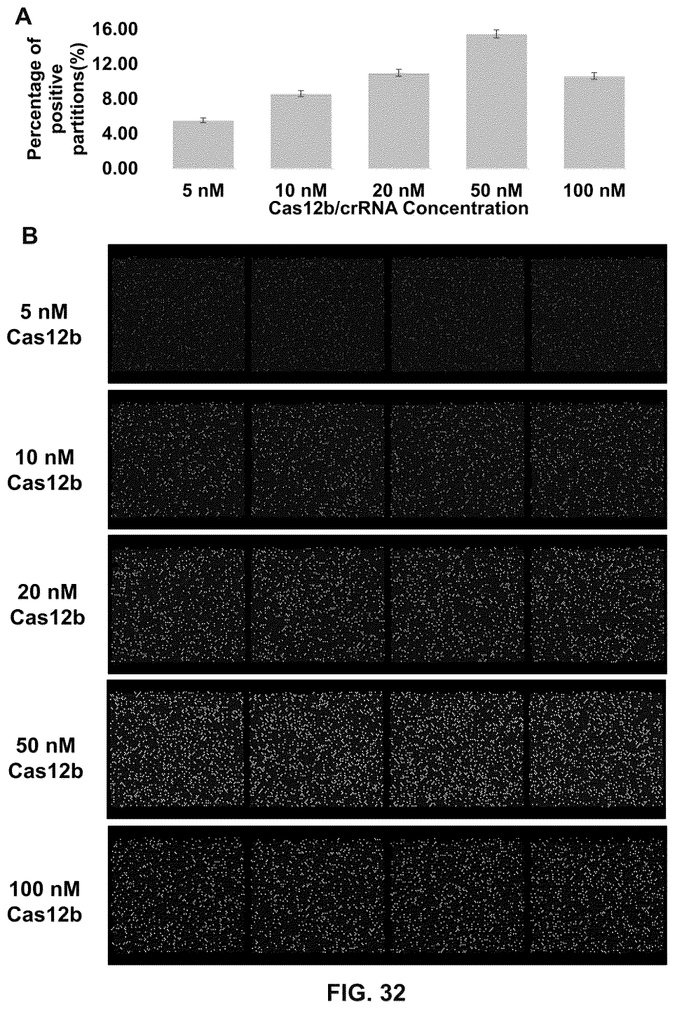

To increase the signal-to-noise ratio (S/N) and reduce the reaction time, the FQ reporter with 5 thymine (T) was chosen because it showed the highest S/N and reaction rate compared to those of other FQ reporters with different base compositions (poly-A, poly-T, poly C, poly G and poly AT, FIG. 25) or different lengths (5-20 nucleotides, FIG. 26) in bulk reaction. Taurine was also added to the reaction as it improved the reaction kinetics (FIG. 27), which is consistent with a previous study [Joung, J. et al. New England Journal of Medicine (2020)]. Also, the concentrations of the enzymes were optimized to get the highest positive partition ratio in the presence of the same concentrations of SARS-CoV-2 synthetic RNA or plasmid containing the SARS-CoV-2 N gene using RADICA. The resulting optimal concentrations for RADICA were 0.3 U/μL WarmStart RTx reverse transcriptase (FIGS. 28 and 29), 0.96 U/μL Bst 2.0 WarmStart DNA polymerase (FIGS. 30 and 31), and 50 nM Cas12b/crRNA (FIG. 32).

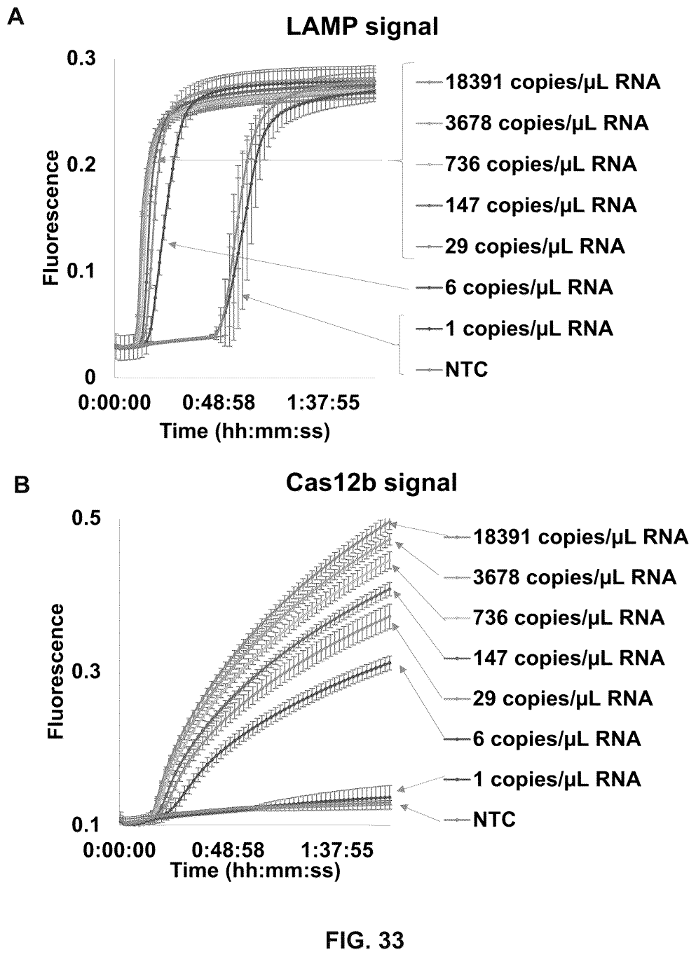

Using the optimized parameters above, we subsequently tested the real-time performance of bulk reactions at concentrations of RNA ranging from 1 to 18391 copies/μL. In the one-pot assay, RT-LAMP reactions were monitored using a SYTO-82 orange fluorescent nucleic acid stain (FIG. 33A), and Cas12b reactions were monitored using a green FQ reporter (FIG. 33B). From the result, the RT-LAMP-Cas12b can detect as little as 6 copies/μL RNA in the bulk reaction, while 1 copy/μL RNA showed no difference to non-targeted control (NTC). From the SYTO-82 fluorescent signal (FIG. 33A), the exponential amplification of target started at 9-15 min and reached a plateau at 25-35 min; the time needed to reach the plateau correlated with the concentration of target RNA. At the time of 47 min, the NTC started to have non-specific amplification signals, which are common in LAMP due to the interaction of multiple primers. Non-template amplification is hard to avoid with LAMP because six primers are involved; thus, an additional CRISPR/Cas-based detection is vital to the specificity of this assay. From the FAM fluorescent signal (FIG. 33B), the trans cleavage of Cas12b started 6 min later than LAMP activity, suggesting that a short time is needed for the amplified DNA to accumulate and for Cas12b to detect. No increased FAM fluorescent signal was detected in the NTC sample, which is different from the LAMP signal and indicates the specificity of the Cas12b reaction. Also, the Cas12b was still proceeding without reaching a plateau even after 2 h of reaction time, indicating that the end-point fluorescence of the bulk reaction may be used as a semi-quantitative marker. These results confirmed the short reaction time of the one-pot RT-LAMP-Cas12b reaction and the specificity of the reaction obtained by adding Cas12b/crRNA.

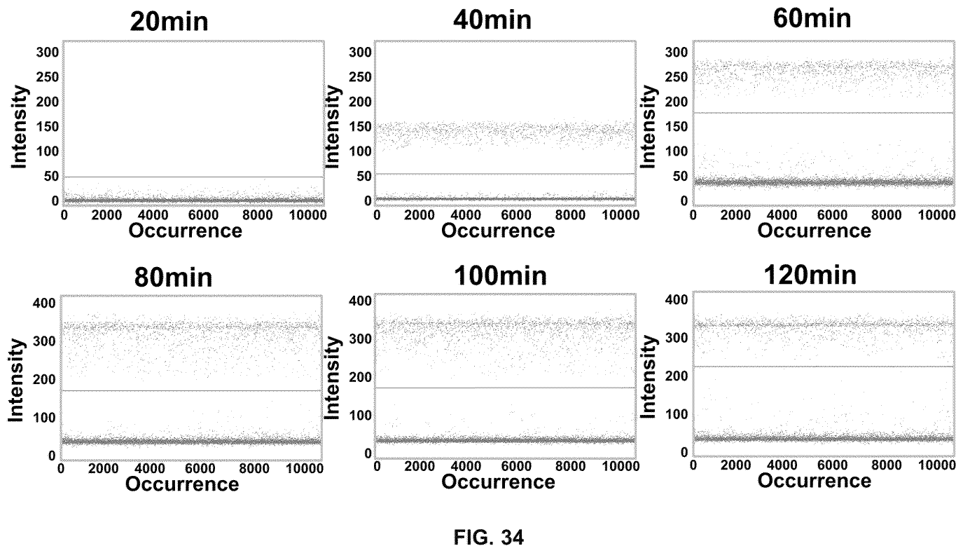

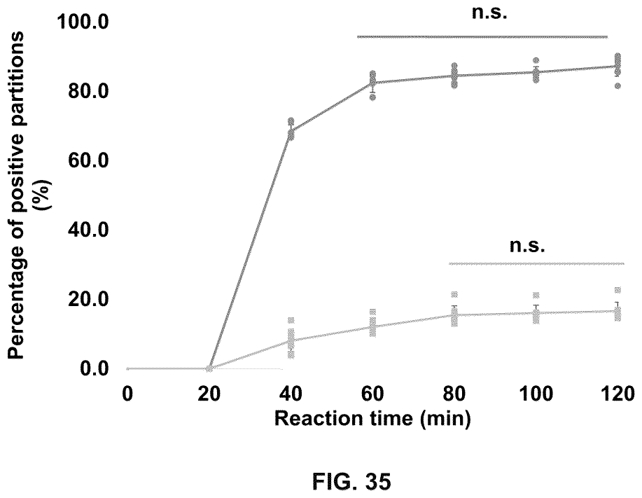

We then investigated the reaction time of the reaction on a digital device by measuring the percentages of positive partitions at different reaction times. From the end-point fluorescence results at different time (FIG. 34), most of the positive partitions could be detected at 40 min for the targets at different concentrations, indicating that 40 min was enough for qualitative detection, which is consistent with the bulk reaction. After 40 min, the fluorescent signals were increasing, which is similar to bulk reaction. To investigate the time-course performance of RADICA, fluorescent signals of the partitions at different target concentrations were measured and the percentage of positive partitions were calculated (FIG. 35). At 40 min, although most of the positive partitions were detected, some partitions containing the targets could not be distinguished from the background and were assigned as negative by the software due to the slightly low fluorescence level. As time went on, fluorescent signals increased in these partitions and the percentage of positive partitions plateaued at 60 min for high concentration sample and 80 min for low concentration sample. After 80 min, the reaction was still proceeding and the fluorescent signals in the positive partitions were still increasing (FIG. 34), while the ratio of positive partitions remained constant (FIG. 35). For the high concentration samples (around 85% of the partitions were positive), some of the positive partitions contain multiple targets based on Poisson distribution, thus making it faster to reach plateau. For low concentration samples (around 15% of the partitions were positive), most of the positive partitions contain one target, which may account for the relatively slow speed compared to high concentration sample. Above all, these results indicate that 40 min incubation is enough for qualitative detection and 60-80 min incubation is enough for quantitative detection by RADICA. As a tradeoff for rapid test, all subsequent experiments were performed for 60 min.

### Example 14

### Performance of RADICA (Digital RT-LAMP-Cas12b) and its Applications on Different Digital Devices

To investigate the performance of RADICA, the specificity, quantification capability, and applicability of this method were evaluated on different devices, using the primers and crRNA shown in Table 7.

TABLE 7
Primers, crRNA for RADICA (digital RT-LAMP-Cas12b) targeting SARS-CoV-2.

| Name | Sequence | Application |
|---|---|---|
| FQ5T | /56-FAM/TTTTT/3IABKFQ/ | FQ reporter |
| N2-LAMP- | GUCUAGAGGACAGAAUUUUUCAACGGG | SARS-CoV-2 |
| crRNA11 | UGUGCCAAUGGCCACUUUCCAGGUGG | crRNA for Cas12b* |
| SEQ ID NO: | CAAAGCCCGUUGAGCUUCUCAAAUCUG | |
| 50 | AGAAGUGGCACCGAAGAACGCUGAAGC | |
| | GCUG | |
| N2-WSLAMP- | GCTGCTGAGGCTTCTAAG | SARS-CoV-2 |
| F3 | | LAMP primers* |
| SEQ ID NO: | | |
| 51 | | |
| N2-WSLAMP- | GCGTCAATATGCTTATTCAGC | SARS-CoV-2 |
| B3 | | LAMP primers* |
| SEQ ID NO: | | |
| 52 | | |
| N2-WSLAMP- | GCGGCCAATGTTTGTAATCAGTAGACGT | SARS-CoV-2 |
| FIP | GGTCCAGAACAA | LAMP primers* |
| SEQ ID NO: | | |
| 653 | | |
| N2-WSLAMP- | TCAGCGTTCTTCGGAATGTCGCTGTGTA | SARS-CoV-2 |
| BIP | GGTCAACCACG | LAMP primers* |
| SEQ ID NO: | | |
| 54 | | |
| N2-WSLAMP- | CCTTGTCTGATTAGTTCCTGGT | SARS-CoV-2 |
| LoopF | | LAMP primers* |
| SEQ ID NO: | | |
| 55 | | |
| N2-WSLAMP- | TGGCATGGAAGTCACACC | SARS-CoV-2 |
| LoopB | | LAMP primers* |
| SEQ ID NO: | | |
| 56 | | |

*Joung, J. et al., New England Journal of Medicine (2020).

### Methods:

### Bulk RT-LAMP-Cas12b Reactions:

The DNA/RNA target samples were mixed with 1.6 μM FIP primers, 1.6 μM BIP primers, 0.2 μM F3 primers, 0.2 μM B3 primers, 0.4 μM LoopF primers, 0.4 μM LoopB primers, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, and 50 nM crRNA in 1× isothermal amplification buffer, unless otherwise indicated. To detect the LAMP signal, 250 mM SYTO-82 fluorescent nucleic acid stain was added to the reaction. The reaction mixture was incubated at 60° C. and fluorescence kinetics were monitored for 1-2 h using Roche Light Cycler 96. The fluorescence values displayed in the results were the fluorescence levels determined by Roche Light Cycler 96 software. To mimic the complexity of real samples, 1 ng/μL human genomic DNA was also added to the reactions.

### RADICA Reactions by Clarity Digital Chip:

RADICA reaction was prepared by adding 1× Clarity JN solution to the RT-LAMP-Cas12b bulk reactions described above and partitioned on Clarity digital chip (˜1.336 nL partition volume, ˜10,000 partitions per reaction). 15 μL reaction mixtures were loaded onto the digital chip followed by treatment with the Clarity sealing enhancer and sealing with 230 μL Clarity sealing fluid. The tube containing the digital chip was warmed in a water bath at for 1 hour, unless otherwise indicated. After incubation, the end-point fluorescence in the 10,000 partitions was detected by Clarity Reader. Using Clarity software, the threshold was determined based on the florescence distribution of the partitions and positive partition percentages and input nucleic acids concentration were then calculated based on the threshold.

### RADICA Reactions by QIAcuity Digital Nanoplate:

The RADICA reaction was prepared by adding 250 nM Cyanine 680 succinimidyl ester (biotium) as reference dye to the RT-LAMP-Cas12b bulk reactions stated above and partitioned on QIAcuity digital nanoplates (˜0.91 nL partition volume, ˜26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

### Results:

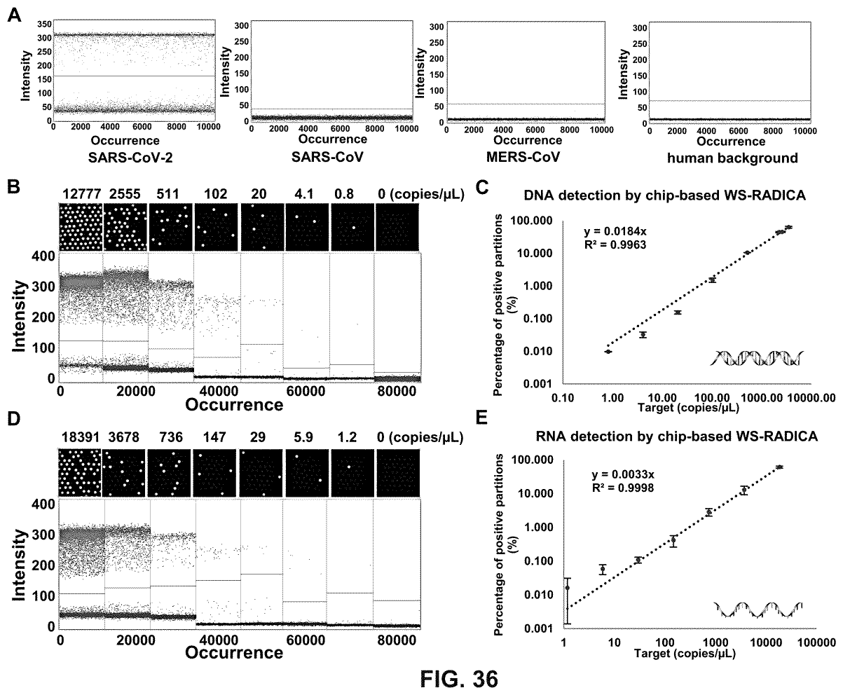

DNA or RNA containing the SARS-CoV-2 sequence was added to the reaction as the target and LAMP primers and crRNA targeting N gene of SARS-CoV-2 were used to evaluate the performance of the digital reaction on commercially available Clarity digital chips (1.336 nL partition volume, ˜10,000 partitions per reaction) [Joung, J. et al. New England Journal of Medicine (2020)]. First, the specificity of RADICA was tested by comparing results of SARS-CoV-2 DNA with SARS-CoV DNA, MERS-CoV DNA, and a human genomic DNA control. From the result (FIG. 36A), positive partitions were detected only in the target sample but not in the controls, indicating the high specificity of this method in distinguishing similar sequences.

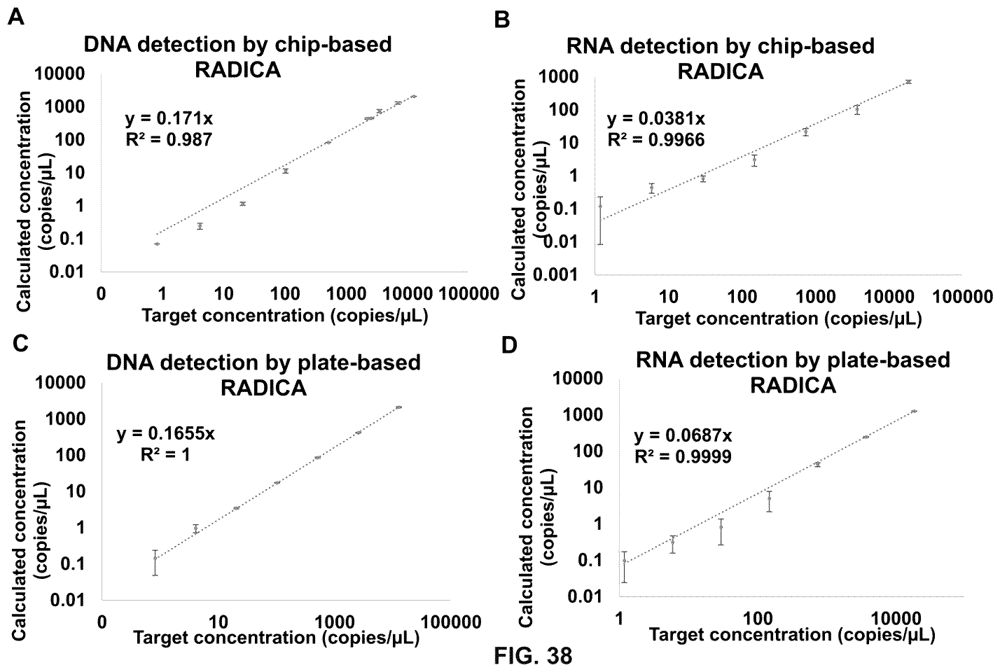

As it would be useful to quantify nucleic acids with this method as well as to detect their presence, RADICA was evaluated for its ability to distinguish different concentrations of SARS-CoV-2 DNA (12777 to 0.8 copies/μL) or RNA (18391 to 1.2 copies/μL). From the results of about 10,000 partitions (FIGS. 36B and 36D), positive partitions decreased in more diluted samples and no positive partitions were detected in NTC. There was a strong linear relationship (R²=0.99) between the target concentrations and the percentage of positive partitions in both DNA and RNA samples (FIGS. 36C and 36E). The calculated concentration of RADICA based on Poisson distribution was lower than the target concentration (FIG. 38), which can be attributed to “molecular dropout” or low filling rate of the microwells, as has been reported for dPCR, dRPA, and dLAMP reactions [Whale, A. S. et al. PloS one 8: e58177 (2013)]. Nevertheless, RADICA has a single reaction temperature and faster reaction time, which are important advantages. Also, the reactions noted above were observed in the presence of human DNA, indicating that RADICA's quantification capability was not affected by background nucleic acids.

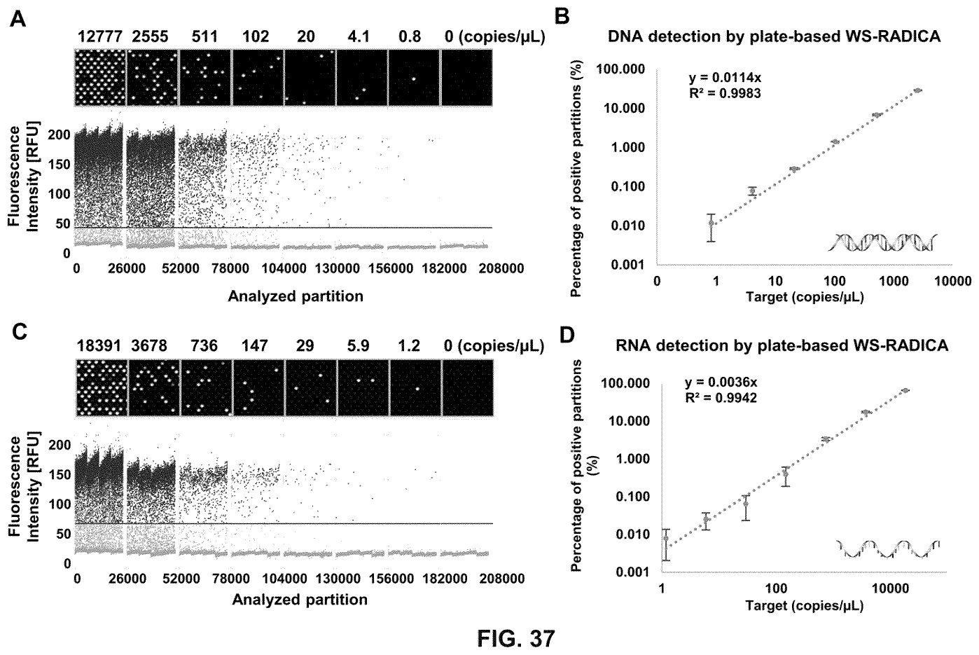

Having shown the specificity and quantification capability on the Clarity digital chip, we determined whether RADICA could be easily used with other digital devices which had been previously designed for digital PCR and which are in common use. A plate-based QIAcuity Digital System was tested for this purpose (˜0.91 nL partition volume, 26,000 partitions per reaction). The same one-pot reactions were digitalized using the sample partition system on QIAcuity to divide the samples into 26,000 individual wells in the plate followed by 60° C. incubation for 60 min. For both DNA (0.8-12777 copies/μL) and RNA (1.2-18391 copies/μL), the number of positive wells in the plate increased when more targets were present (FIGS. 37A and 37C). A strong linear relationship (R²=0.99) was also found between the target concentration and the positive well ratio (FIGS. 37B and 37D). Although the relationship between target concentration and positive partition percentage was slightly different for the two digital devices due to their different partition volumes, accurate quantification results were obtained based on the standard curve from the same device. Therefore, RADICA can be easily adapted to different digital devices, offering a faster alternative for those who already have digital PCR machines for nucleic acid quantification.

### Example 15

Comparison of RADICA (Digital RT-LAMP-Cas12b) with Other Detection Methods

To determine whether RADICA is competitive with other nucleic acid detection methods, such as RT-qPCR, RT-dPCR, and RT-LAMP-Cas12b bulk assay, we performed these methods and compared their detection of the same concentrations of SARS-CoV-2 RNA in the presence of a human genomic DNA background.

### Methods:

### RT-qPCR Reaction:

Serial dilutions of RNA targets were mixed with 1×USCDC N2 assay, 1× TaqMan™ Fast Virus 1-Step Master Mix and loaded on Roche Light Cycler. The reactions were incubated at 55° C. for 5 min followed by 95° C. for 20 s (one cycle), 95° C. 10 s and 60° C. 30 s (45 cycles). Fluorescent signals were monitored and Cq (Ct) values were calculated automatically using Roche Light Cycler 96 software.

DNA Quantification by dPCR:

Plasmids containing the N genes of SARS-CoV-2, SARS-CoV, and MERS-CoV were linearized with FastDigest Scal (Thermo Scientific) then mixed with 1×USCDC N2 assay, lx TaqMan™ Fast Advanced Master Mix (Applied Biosystems) and 1× Clarity™ JN solution (JN Medsys). The 15 μL reactions were loaded onto the Clarity digital chip using the method mentioned above, and the tubes containing the digital chip were transferred to a PCR machine using the following parameters (ramp rate=1° C./s): 95° C. for 15 min (one cycle), 95° C. for 50 s and 58° C. for 90 s (40 cycles), and 70° C. for 5 min. The endpoint fluorescence of the partitions was detected with a Clarity™ Reader, and the input DNA copy numbers were calculated by Clarity™ software.

### RNA Detection by RT-dPCR:

Serial dilutions of RNA targets were mixed with 1×USCDC N2 assay, 1× TaqMan™ Fast Virus 1-Step Master Mix (Applied Biosystems) and 1× Clarity™ JN solution (JN Medsys) followed by incubation at 55° C. 5 min. After incubation, the reactions were partitioned on the Clarity digital chip and transferred to a PCR thermocycler with the following parameters (ramp rate=1° C./s): 95° C. for 15 min (one cycle), 95° C. for 50 s and 58° C. for 90 s (40 cycles), and 70° C. for 5 min. The endpoint fluorescence of the partitions was detected using a Clarity™ Reader and the input RNA copy numbers were calculated by Clarity™ software.

### Inhibitor Tolerance Test of the Reactions:

To test the inhibitor tolerance, 2.5 U/mL heparin, 0.01% sodium dodecyl sulphate (SDS), 1 mM ethylenediaminetetraacetic acid (EDTA), or 5% ethanol was added to the reactions mentioned above. For bulk reactions (RT-qPCR and bulk RT-LAMP-Cas12b), the 1 h end-point fluorescent signals of reactions with inhibitors were divided by the reactions without inhibitors. For digital reactions (RT-dPCR and RADICA), the positive partition percentage of the reactions with inhibitors was divided by that of the reactions without inhibitors, and the corresponding ratio was taken as the percentage of efficiency.

### Results:

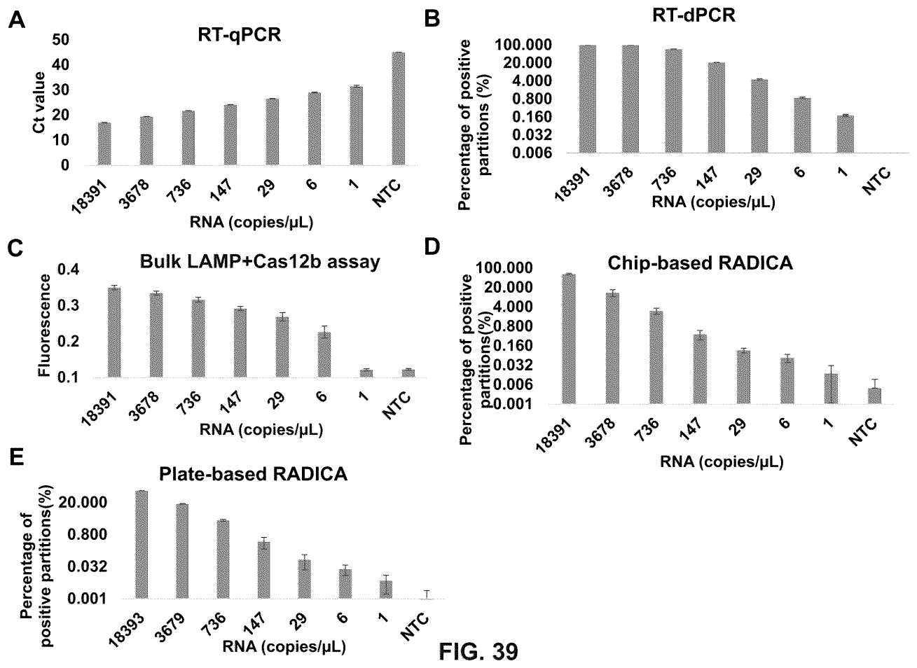

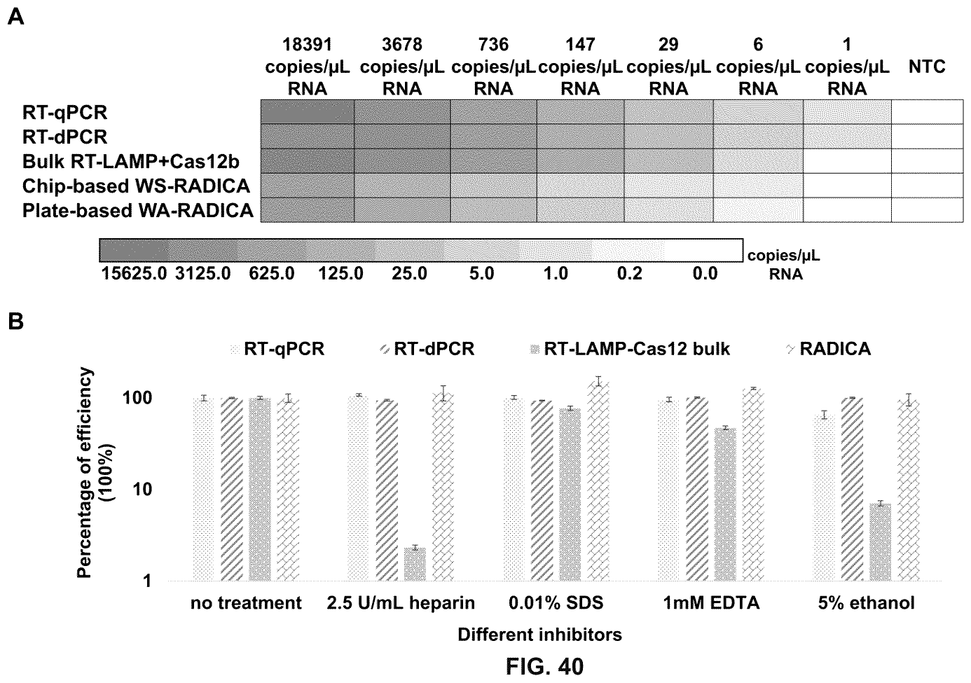

To determine whether RADICA is competitive with other nucleic acid detection methods, such as RT-qPCR, RT-dPCR, and RT-LAMP-Cas12b bulk assay, we performed the above four methods and compared their detection of the same concentrations of SARS-CoV-2 RNA (18391 to 1 copies/μL) in the presence of a human genomic DNA (1 ng/A) background (FIGS. 39 and 40). For PCR-based experiments (RT-qPCR and RT-dPCR), the FDA EUA-approved CDC assay targeting the SARS-CoV-2 N gene was used; and for the CRISPR based methods (RT-LAMP-Cas12b bulk reaction and RADICA), primers and crRNA targeting the same N gene region of SARS-CoV-2 were used [Joung, J. et al. New England Journal of Medicine (2020)]. For RT-qPCR, the serial dilutions of SARS-CoV-2 RNA gave threshold cycle numbers (Ct) ranging from 17.8 to 32.2, and there was a very strong linear relationship between Ct and the log of target concentration, as well as sensitivity corresponding to the detection of 1 copy/μL (FIG. 39). For RT-dPCR, the RNA concentration (18391 copies/μL) was far above the upper linear range and resulted in more than 99.6% of the partitions being positive. A good linear relationship between the target concentration and measured concentration was also observed for the RT-dPCR results, corresponding to the detection of a large range of concentrations, from 3678 copies/μL to 1 copy/μL of RNA (FIG. 39). However, for RT-dPCR the reaction took much longer (more than 3 h), which may hinder its widespread use.

Compared to PCR-based methods, RT-LAMP-Cas12b-based method is relatively simple, as isothermal amplification does not require thermal cyclers. As to sensitivity, the bulk reaction was slightly weaker in detecting low copy number samples, resulting in a detection sensitivity of 6 copies/μL RNA (FIG. 39). Although end-point fluorescence of the bulk reaction can be used as a semi-quantitative marker, the relatively weak linear relationship between signal and target suggests that the RT-LAMP-Cas12b bulk reaction is not suitable for quantification. With RADICA, the whole reaction is divided into thousands of independent reactions, calculating the percentage of positive partitions enables quantification. Also, the sensitivity of the reaction increased by taking the benefit of confinement effect on local concentration. One molecule confined in a 1 nL microwell equals to 1.66 fM local concentration, which also equals to 10,000 molecules in a 10 μL bulk reaction. Thus, we still see positive partitions in RADICA at 1 copy/μL but not in the bulk reactions (FIG. 39). Moreover, RADICA widened linear dynamic range for quantitative detection beyond that achieved with RT-dPCR and enabled the quantification of 18391 copies/μL RNA on the same digital device. Overall, RADICA has better sensitivity and quantitation ability than the bulk reaction and higher speed and wider dynamic range compared to RT-dPCR, which make it a promising alternative for nucleic acid quantification.

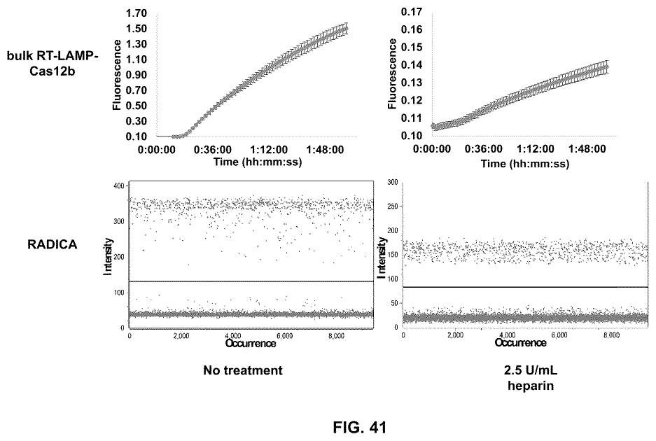

As inhibitors in the samples are likely to alter the reaction and might affect the accuracy of the result, we tested the effect of various inhibitors on the above four detection methods (FIG. 40B). Heparin, normally used as an anticoagulant in blood, serum, or plasma, was found in a previous study to act on DNA polymerase and thus inhibit the reaction. In our study, 2.5 U/mL heparin had no effect on RT-qPCR or RT-dPCR but greatly inhibited the bulk RT-LAMP-Cas12b reaction. In contrast, RADICA tolerated heparin, showing similar quantitative results in the reactions with or without heparin. SDS, an ionic detergent commonly used in sample lysis, was also reported as a DNA polymerase inhibitor. SDS slightly inhibited the bulk RT-LAMP-Cas12b reaction, whereas RADICA recovered the reaction. Similarly, EDTA, which chelates metal ions, such as Mg²⁺, was found to inhibit the bulk RT-LAMP-Cas12b reaction but did not inhibit RADICA. Also, ethanol, which is always used in nucleic acid purification, was tested for its effects on the four different methods. Both bulk RT-qPCR and the bulk RT-LAMP-Cas12b reaction were inhibited by ethanol to some degree. In contrast, the digital reactions (RT-dPCR and RADICA) were more tolerant of ethanol. In accordance with previous studies on digital PCR [Dingle, T. C. et al. Clinical chemistry 59: 1670-1672 (2013)], our experiments showed that digital reactions, not only RT-dPCR but also RADICA, are more robust and less susceptible to inhibitors than bulk reactions, possibly because the individual micro-reactions alleviate the effect of inhibitors as long as the fluorescent signal can be differentiated from background in the presence of inhibitory substances (FIG. 41) [Dingle, T. C. et al. Clinical chemistry 59: 1670-1672 (2013)]. Thus, the digital detection format of the CRISPR reaction offers great advantages not only in sensitivity but also in inhibitor tolerance.

### Example 16

### Performance of RADICA (Digital RT-LAMP-Cas12b) on Virus

After the validation of RADICA on SARS-CoV-2 sequence, we asked whether the method can be adapted to detect and quantify other targets. Biomanufacture for protein therapeutics, vaccines and cell therapy usually need cell culture, which is susceptible to contamination with viruses. Thus, a rapid quantification method is needed to monitor the virus contaminations in biomanufacturing processes. To make use of the speed and quantification ability of RADICA, we have designed RADICA assays to detect and quantify the most common viral contaminants in biomanufacture: human adenovirus and herpes simplex virus [Barone, P. W. et al. Nature biotechnology 38: 563-572 (2020)]. Different LAMP primer sets and crRNA were designed (Table 8) and screened by bulk LAMP-Cas12b reaction, and the combination of LAMP primer set and crRNA with the highest speed were selected for the following RADICA experiment.

TABLE 8
Primers, crRNA for RADICA (digital RT-LAMP-Cas12b) targeting HSV
and hAdV.

| Name | Sequence | Application |
|---|---|---|
| FQ5T | /56-FAM/TTTTT/3IABKFQ/ | FQ reporter |
| HSV1-UL30-F3 | rCCGACGTGTACTAyTACGA | HSV LAMP primer |
| SEQ ID NO: 57 | | |
| HSV1-UL30-B3 | GGyTGGGCyrGCGyGTT | HSV LAMP primer |
| SEQ ID NO: 58 | | |
| HSV1-UL30-FIP | sGGGCArAAGTTGTCGCACA- | HSV LAMP primer |
| SEQ ID NO: 59 | TACCGCGTCTwCGTsCG | |
| HSV1-UL30-BIP | TCArGAAGTACGAGGGKGGsGT- | HSV LAMP primer |
| SEQ ID NO: 60 | CGGTACCAGCCGAAGGT | |
| HSV1-UL30-LF | GTAsGyCAGCKCGCGCCCGCT | HSV LAMP primer |
| SEQ ID NO: 61 | | |
| HSV1-UL30-LB | CCGGTTyATCCTGGACAACC | HSV LAMP primer |
| SEQ ID NO: 62 | | |
| HSV2-UL30-F3 | CCmCCGTCACCGTCTTyCA | HSV LAMP primer |
| SEQ ID NO: 63 | | |
| HSV2-UL30-B3 | TCvACCTCCKCCTTGTTCA | HSV LAMP primer |
| SEQ ID NO: 64 | | |
| HSV2-UL30-FIP | AAyCGCKCGTGGArCTGGG- | HSV LAMP primer |
| SEQ ID NO: 65 | CGTGTAyGACATCCTGGAG | |
| HSV2-UL30-BIP | GGACCGTCATCACGCTyCTG- | HSV LAMP primer |
| SEQ ID NO: 66 | GCGTGCCGTArACGTGAAC | |
| HSV2-UL30-LF | CGCATGCyGTACGCGTG | HSV LAMP primer |
| SEQ ID NO: 67 | | |
| HSV2-UL30-LB | GGyCTGACyCCsGAAGGCCA | HSV LAMP primer |
| SEQ ID NO: 68 | | |
| HSV3-UL30-F3 | CGCGTCTwCGTsCGAAGC | HSV LAMP primer |
| SEQ ID NO: 69 | | |
| HSV3-UL30-B3 | GGyTGGGCyrGCGyGTT | HSV LAMP primer |
| SEQ ID NO: 70 | | |
| HSV3-UL30-FIP | GACsCCmCCCTCGTACTTCyTG- | HSV LAMP primer |
| SEQ ID NO: 71 | CGCGyGCTGkCsTACC | |
| HSV3-UL30-BIP | CGCCACCACCCGGTTyAT- | HSV LAMP primer |
| SEQ ID NO: 72 | GCCsGGyTTGAGrCGGTAC | |
| HSV3-UL30-LF | GGGCAGAAGTTGTCGCACA | HSV LAMP primer |
| SEQ ID NO: 73 | | |
| HSV3-UL30-LB | GGGTTyGTCACCTTCGGCTG | HSV LAMP primer |
| SEQ ID NO: 74 | | |
| HSV4-UL30-F3 | rCACGCGTACrGCATG | HSV LAMP primer |
| SEQ ID NO: 75 | | |
| HSV4-UL30-B3 | CGCTCGCAGAGATCKCG | HSV LAMP primer |
| SEQ ID NO: 76 | | |
| HSV4-UL30-FIP | AGrCCCAGrAGCGTGATGA- | HSV LAMP primer |
| SEQ ID NO: 77 | CACGmGCGrTTTATGGAC | |
| HSV4-UL30-BIP | SGCCGTTCACGTyTACGGCA- | HSV LAMP primer |
| SEQ ID NO: 78 | GCvCGGCAyTGyAGGTG | |
| HSV4-UL30-LF | GTCCCsGysGGyGTGAT | HSV LAMP primer |
| SEQ ID NO: 79 | | |
| HSV4-UL30-LB | GCGGCAGTACTTTTACATGAACAAG | HSV LAMP primer |
| SEQ ID NO: 80 | | |
| HSV5-UL30-F3 | CGTCTTyCACGTGTAyGACA | HSV LAMP primer |
| SEQ ID NO: 81 | | |
| HSV5-UL30-B3 | GGCvCGGCAyTGyAGG | HSV LAMP primer |
| SEQ ID NO: 82 | | |
| HSV5-UL30-FIP | CCATAAAyCGCKCGTGGArCT- | HSV LAMP primer |
| SEQ ID NO: 83 | CTGGAGmACGTGGArCAC | |
| HSV5-UL30-BIP | CTGACyCCsGAAGGCCAyC- | HSV LAMP primer |
| SEQ ID NO: 84 | CvACCTCCKCCTTGTTCATG | |
| HSV5-UL30-LF | GCGCGCATGCyGTACGC | HSV LAMP primer |
| SEQ ID NO: 925 | | |
| HSV5-UL30-LB | CGGCACGCGGCAGTACTTT | HSV LAMP primer |
| SEQ ID NO: 86 | | |
| HSV6-UL30-F3 | AGATGyTGTTGGCCTTCATG | HSV LAMP primer |
| SEQ ID NO: 87 | | |
| HSV6-UL30-B3 | AAGTGGCTCTGGCCKATG | HSV LAMP primer |
| SEQ ID NO: 88 | | |
| HSV6-UL30-FIP | CAGCTTGGyCAGsAyGAAGGG- | HSV LAMP primer |
| SEQ ID NO: 89 | AArCAGTACGGCCCCGAG | |
| HSV6-UL30-BIP | tgACGGAsATyTACAAGGTCCC- | HSV LAMP primer |
| SEQ ID NO: 90 | tCCCACACGCGrAACAC | |
| HSV6-UL30-LF | TGATGATGTTGTACCCGGTCACGA | HSV LAMP primer |
| SEQ ID NO: 91 | | |
| HSV6-UL30-LB | TACGGSCGCATGAACGGCCG | HSV LAMP primer |
| SEQ ID NO: 92 | | |
| HSV7-UL30-F3 | GGyGTGTTyCGCGTGTG | HSV LAMP primer |
| SEQ ID NO: 93 | | |
| HSV7-UL30-B3 | ATCACCCCGCGyTGCG | HSV LAMP primer |
| SEQ ID NO: 94 | | |
| HSV7-UL30-FIP | TGTTCACCATsCCGTTCACCTT- | HSV LAMP primer |
| SEQ ID NO: 95 | GGACATmGGCCAGAGCCAC | |
| HSV7-UL30-BIP | TCSAGCTACAAGCTSAACGCC- | HSV LAMP primer |
| SEQ ID NO: 96 | GCGGGGATGTCGCGrTAG | |
| HSV7-UL30-LF | TCTTGCTGCGCTTCTGrAA | HSV LAMP primer |
| SEQ ID NO: 97 | | |
| HSV7-UL30-LB | GTCyTGAAGGACAAGAAGAAGGA | HSV LAMP primer |
| SEQ ID NO: 98 | | |
| HSV8-UL30-F3 | GCCGTCyTGAAGGACAAGA | HSV LAMP primer |
| SEQ ID NO: 99 | | |
| HSV8-UL30-B3 | rATrCCCGCCArGCGC | HSV LAMP primer |
| SEQ ID NO: 100 | | |
| HSV8-UL30-FIP | TACTCGCCGATCACCCCGC- | HSV LAMP primer |
| SEQ ID NO: 101 | AGAAGGAyCTGAGCTAyCGC | |
| HSV8-UL30-BIP | CAGGAyTCsCTGCTGGTsGG- | HSV LAMP primer |
| SEQ ID NO: 102 | GGCSGArAGCTCCAGrTG | |
| HSV8-UL30-LF | GCGTAGTAGGCGGGGATGT | HSV LAMP primer |
| SEQ ID NO: 103 | | |
| HSV8-UL30-LB | CAGCTGTTTTTTAAGTTT | HSV LAMP primer |
| SEQ ID NO: 104 | | |
| HSV9-UL30-F3 | GCCTTCATGACCyTyGTsAA | HSV LAMP primer |
| SEQ ID NO: 105 | | |
| HSV9-UL30-B3 | GCTGCGCTTCTGrAAGTG | HSV LAMP primer |
| SEQ ID NO: 106 | | |
| HSV9-UL30-FIP | CAGCTTGGyCAGsAmGAAGGG- | HSV LAMP primer |
| SEQ ID NO: 107 | GGCCCCGAGTTCGTGA | |
| HSV9-UL30-BIP | ACGGASATyTACAAGGTCCCsC- | HSV LAMP primer |
| SEQ ID NO: 108 | TGGCCKATGTCCCACACG | |
| HSV9-UL30-LF | CCAGTCGAAGTTGATGATGTTGTAC | HSV LAMP primer |
| SEQ ID NO: 109 | | |
| HSV9-UL30-LB | GTACGGsCGCATGAACG | HSV LAMP primer |
| SEQ ID NO: 110 | | |
| HSV10-UL30-F3 | yCAGAAGCGCAGCAAGA | HSV LAMP primer |
| SEQ ID NO: 111 | | |
| HSV10-UL30-B3 | CsACCAGCAGsGArTCCT | HSV LAMP primer |
| SEQ ID NO: 112 | | |
| HSV10-UL30- | GGCGTTsAGCTTGTAGCTSGAG- | HSV LAMP primer |
| FIP | TGAACGGSATGGTGAACAT | |
| SEQ ID NO: 113 | | |
| HSV10-UL30- | yCTGAGCTAyCGCGACATCCC- | HSV LAMP primer |
| BIP | TACTCGCCGATCACCCCG | |
| SEQ ID NO: 114 | | |
| HSV10-UL30-LF | CGGTKATrATsCCGTACATGTCG | HSV LAMP primer |
| SEQ ID NO: 115 | | |
| HSV10-UL30-LB | CTACGCCKCCGGGCCCGCGCA | HSV LAMP primer |
| SEQ ID NO: 116 | | |
| HSV11-UL30-F3 | sCGACGGCTGTTCTTCG | HSV LAMP primer |
| SEQ ID NO: 117 | | |
| HSV11-UL30-B3 | CACyCCsGTGAACCCGT | HSV LAMP primer |
| SEQ ID NO: 118 | | |
| HSV11-UL30- | ttTCGCATGGCSAGCCAGTC- | HSV LAMP primer |
| FIP | SAAGGCyCACGTrCGmG | |
| SEQ ID NO: 119 | | |
| HSV11-UL30- | CGGATyCCCCAGAGCASCCC- | HSV LAMP primer |
| BIP | ACCGAGTTrCACACsACCTT | |
| SEQ ID NO: 120 | | |
| HSV11-UL30-LF | AGGATGCTSAGSAGGC | HSV LAMP primer |
| SEQ ID NO: 121 | | |
| HSV11-UL30-LB | AGGCCGTsCTCCTSGACAA | HSV LAMP primer |
| SEQ ID NO: 122 | | |
| HSV12-UL30-F3 | TCTTCGTsAAGGCyCACGT | HSV LAMP primer |
| SEQ ID NO: 123 | | |
| HSV12-UL30-B3 | TGAACCCGTACACCGAGTT | HSV LAMP primer |
| SEQ ID NO: 124 | | |
| HSV12-UL30- | AGCGGATCTGCTTTCGCATGG- | HSV LAMP primer |
| FIP | CGmGAGAGCCTsCTsAGC | |
| SEQ ID NO: 125 | | |
| HSV12-UL30- | CGGATyCCCCAGAGCAsCC- | HSV LAMP primer |
| BIP | CACSACCTTGATGGCGG | |
| SEQ ID NO: 126 | | |
| HSV12-UL30-LF | AGCCAGTCsCGCAGsAGGAT | HSV LAMP primer |
| SEQ ID NO: 127 | | |
| HSV12-UL30-LB | GCCGTsCTCCTSGACAAGCA | HSV LAMP primer |
| SEQ ID NO: 128 | | |
| HSV13-UL30-F3 | GGCTsGCCATGCGAAAG | HSV LAMP primer |
| SEQ ID NO: 129 | | |
| HSV13-UL30-B3 | GTCGCGAGSAGCATCTC | HSV LAMP primer |
| SEQ ID NO: 130 | | |
| HSV13-UL30- | TGCTTGTCSAGGAGSACGG- | HSV LAMP primer |
| FIP | CAGATCCGCTCGCGGATyC | |
| SEQ ID NO: 131 | | |
| HSV13-UL30- | sGTGTGyAACTCGGTGTACGGG- | HSV LAMP primer |
| BIP | TCACSGTsGCGGCmAC | |
| SEQ ID NO: 132 | | |
| HSV13-UL30-LF | TCCTCGGGGSTGCTCTGGG | HSV LAMP primer |
| SEQ ID NO: 133 | | |
| HSV13-UL30-LB | TTCACsGGrGTGCAGCA | HSV LAMP primer |
| SEQ ID NO: 134 | | |
| HSV14-UL30-F3 | GATGGCGAGCCACATCTC | HSV LAMP primer |
| SEQ ID NO: 135 | | |
| HSV14-UL30-B3 | GGTTGATrAACGCGCAGTTG | HSV LAMP primer |
| SEQ ID NO: 136 | | |
| HSV14-UL30- | GCGATbAGCAGCAGCTTGGT- | HSV LAMP primer |
| FIP | ctGTTyCTsCCCCCsATCAA | |
| SEQ ID NO: 137 | | |
| HSV14-UL30- | GTACATCGGCGTCATCTrCGGG- | HSV LAMP primer |
| BIP | TTTTGCGCACCAGrTCsAC | |
| SEQ ID NO: 138 | | |
| HSV14-UL30-LF | GAACGTyTTTTCGCACTCGA | HSV LAMP primer |
| SEQ ID NO: 139 | | |
| HSV14-UL30-LB | GGyAAGATGCTCATyAAGGGC | HSV LAMP primer |
| SEQ ID NO: 140 | | |
| HSV15-UL30-F3 | CCCACyTCCGGGTTTCAC | HSV LAMP primer |
| SEQ ID NO: 141 | | |
| HSV15-UL30-B3 | GTGrGCCTTsACGAAGAACA | HSV LAMP primer |
| SEQ ID NO: 142 | | |
| HSV15-UL30- | AGGTTGTGGGCCTGGATGATG- | HSV LAMP primer |
| FIP | ACCCCGTGGTGGTGTT | |
| SEQ ID NO: 143 | | |
| HSV15-UL30- | GTGCTTCAGyACGCTCTCCC- | HSV LAMP primer |
| BIP | CACCTCGATCTCCAGGTAGT | |
| SEQ ID NO: 144 | | |
| HSV15-UL30-LF | GGGGTACAGGCTGGCAAAGT | HSV LAMP primer |
| SEQ ID NO: 145 | | |
| HSV15-UL30-LB | GTSGCGCACCTGGAGGCGG | HSV LAMP primer |
| SEQ ID NO: 146 | | |
| HSV16-UL30-F3 | GTGACrTTCAAGGCCCTG | HSV LAMP primer |
| SEQ ID NO: 147 | | |
| HSV16-UL30-B3 | mGGGACATCAGCTTCGA | HSV LAMP primer |
| SEQ ID NO: 148 | | |
| HSV16-UL30- | GCCACryyTCGGGAATAAACC- | HSV LAMP primer |
| FIP | TTTGGrAATAACGCCAAGA | |
| SEQ ID NO: 149 | | |
| HSV16-UL30- | GCGCTACGGCGGAGGAAACT- | HSV LAMP primer |
| BIP | CTCATGCTAGAGTATCAAAGGCT | |
| SEQ ID NO: 150 | | |
| HSV16-UL30-LF | TTTAACAGACTCTCGGTGA | HSV LAMP primer |
| SEQ ID NO: 151 | | |
| HSV16-UL30-LB | CGTCGAATGTTGCATAG | HSV LAMP primer |
| SEQ ID NO: 152 | | |
| adv-hexon1-F3 | CARTGGKCDTACATGCA | hAdv LAMP primer |
| SEQ ID NO: 153 | | |
| adv-hexon1-B3 | GTGWASCGMRCYTTGT | hAdv LAMP primer |
| SEQ ID NO: 154 | | |
| adv-hexon1-FIP | AAGTASGTRTCKGTGGCRCGCAGGAY | hAdv LAMP primer |
| SEQ ID NO: 155 | GCYTCGGAGT | |
| adv-hexon1-BIP | ACSCACGATGTGACCACCGRTCMACG | hAdv LAMP primer |
| SEQ ID NO: 156 | GGSAYRAA | |
| adv-hexon1-LF | GCRAACTGCACCAGMCC | hAdv LAMP primer |
| SEQ ID NO: 157 | | |
| adv-hexon1-LB | CAGCGDCTGAYGCTGCG | hAdv LAMP primer |
| SEQ ID NO: 158 | | |
| adv-hexon2-F3 | CGATGMTGCCSCARTGG | hAdv LAMP primer |
| SEQ ID NO: 159 | | |
| adv-hexon2-B3 | RAASCGCAGCRTCAG | hAdv LAMP primer |
| SEQ ID NO: 160 | | |
| adv-hexon2-FIP | AACTGCACCAGMCCSGGRCCDTACAT | hAdv LAMP primer |
| SEQ ID NO: 161 | GCACATCKCSG | |
| adv-hexon2-BIP | CMGAYACSTACTTCARYCTGTCSGTG | hAdv LAMP primer |
| SEQ ID NO: 162 | GTCACATCGTG | |
| adv-hexon2-LF | AGGTACTCCGARGCRTCCTG | hAdv LAMP primer |
| SEQ ID NO: 163 | | |
| adv-hexon2-LB | AACAAGTTTAGRAACCCCA | hAdv LAMP primer |
| SEQ ID NO: 164 | | |
| adv-hexon3-F3 | GGCTGGTGCAGTTYGC | hAdv LAMP primer |
| SEQ ID NO: 165 | | |
| adv-hexon3-B3 | CACRCGGTTRTCRCCCAC | hAdv LAMP primer |
| SEQ ID NO: 166 | | |
| adv-hexon3-FIP | TCGTGCGTRGGYGCCACCGCGCCAC | hAdv LAMP primer |
| SEQ ID NO: 167 | CGAGASSTACT | |
| adv-hexon3-BIP | GGTCYCAGCGYYTGACGCTGCGGTRA | hAdv LAMP primer |
| SEQ ID NO: 168 | ACCGCGCYTTGT | |
| adv-hexon3-LF | GGGTTYCTAAACTTGTTAYTCAGGC | hAdv LAMP primer |
| SEQ ID NO: 169 | | |
| adv-hexon3-LB | GGTTYATCCCYGTGGACCG | hAdv LAMP primer |
| SEQ ID NO: 170 | | |
| adv-hexon4-F3 | TGGCKACCCCWTCGATGA | hAdv LAMP primer |
| SEQ ID NO: 171 | | |
| adv-hexon4-B3 | CGCTGRGACCGGTCKGT | hAdv LAMP primer |
| SEQ ID NO: 172 | | |
| adv-hexon4-FIP | AGCCCGGGGCTCAGGTACTCCCGCA | hAdv LAMP primer |
| SEQ ID NO: 173 | GTGGTCKTACATGC | |
| adv-hexon4-BIP | CGCGCCACCGAGASSTACTGGTYACR | hAdv LAMP primer |
| SEQ ID NO: 174 | TCGTGCGTRGG | |
| adv-hexon4-LF | CGAGGCGTCCTGGCCCGAGA | hAdv LAMP primer |
| SEQ ID NO: 175 | | |
| adv-hexon4-LB | GTTTAGRAACCCCACGGTGG | hAdv LAMP primer |
| SEQ ID NO: 176 | | |
| adv-hexon5-F3 | GACCTGGGYCARAACCT | hAdv LAMP primer |
| SEQ ID NO: 177 | | |
| adv-hexon5-B3 | GTTGCCRGCCGAGAAGG | hAdv LAMP primer |
| SEQ ID NO: 178 | | |
| adv-hexon5-FIP | CTCGTCCATGGGRTCSACCTCKCTCTA | hAdv LAMP primer |
| SEQ ID NO: 179 | YGCMAACTCCGCC | |
| adv-hexon5-BIP | TYGAAGTCTTTGACGTGGTCCGGGTA | hAdv LAMP primer |
| SEQ ID NO: 180 | CACGGTYTCGATGAC | |
| adv-hexon5-LF | AARGTCATRTCKAGCGCGTG | hAdv LAMP primer |
| SEQ ID NO: 181 | | |
| adv-hexon5-LB | GTSCACCAGCCGCACCGCGGC | hAdv LAMP primer |
| SEQ ID NO: 182 | | |
| adv-hexon6-F3 | ACGCACGAYGTRACCAC | hAdv LAMP primer |
| SEQ ID NO: 183 | | |
| adv-hexon6-B3 | GTGCCRGAGTAGGGCTTRAA | hAdv LAMP primer |
| SEQ ID NO: 184 | | |
| adv-hexon6-FIP | GCRGTRTCCTCRCGGTCCACGACCGG | hAdv LAMP primer |
| SEQ ID NO: 185 | TCYCAGCGYYTG | |
| adv-hexon6-BIP | TCGTACAARGCGCGGTTYACCCCGCC | hAdv LAMP primer |
| SEQ ID NO: 186 | GCGRATGTCAAAGT | |
| adv-hexon6-LF | RGGGATRAACCGCAGCGT | hAdv LAMP primer |
| SEQ ID NO: 187 | | |
| adv-hexon6-LB | GTGGGYGAYAACCGYGTGC | hAdv LAMP primer |
| SEQ ID NO: 188 | | |
| adv-hexon7-F3 | RTAYYTGCCHGACAAGCT | hAdv LAMP primer |
| SEQ ID NO: 189 | | |
| adv-hexon7-B3 | GAGCGRTARCGBAGGCC | hAdv LAMP primer |
| SEQ ID NO: 190 | | |
| adv-hexon7-FIP | ARTCYACHAYCCCVGGRGCCGTRRAA | hAdv LAMP primer |
| SEQ ID NO: 191 | ATHTCTSMYAACCC | |
| adv-hexon7-BIP | GCTACATYAACCTKGGVGCRCGMGCA | hAdv LAMP primer |
| SEQ ID NO: 192 | TTGCGGTGRTGGT | |
| adv-hexon7-LF | ACCACTCGCTTGTTCAT | hAdv LAMP primer |
| SEQ ID NO: 193 | | |
| adv-hexon7-LB | GACTAYATGGACAACGTYAAYCC | hAdv LAMP primer |
| SEQ ID NO: 194 | | |
| adv-hexon8-F3 | GTKGACGGRGCYAGCATYA | hAdv LAMP primer |
| SEQ ID NO: 195 | | |
| adv-hexon8-B3 | GGGATRGAKATGGGSACGT | hAdv LAMP primer |
| SEQ ID NO: 196 | | |
| adv-hexon8-FIP | AGCGTKGAGGCSGTGTTGTGGABAGC | hAdv LAMP primer |
| SEQ ID NO: 197 | ATYTGYCTYTACGC | |
| adv-hexon8-BIP | AGRAAYGACACCAACGACCAGTGGGK | hAdv LAMP primer |
| SEQ ID NO: 198 | ATRGGRTADAGCATGT | |
| adv-hexon8-LF | GGCCATSGGRAARAAGGTGG | hAdv LAMP primer |
| SEQ ID NO: 199 | | |
| adv-hexon8-LB | GACTAYCTHTCCGCCGCCA | hAdv LAMP primer |
| SEQ ID NO: 200 | | |
| adv9-F3 | CGCCTCTGCGTGAAGAC | hAdv LAMP primer |
| SEQ ID NO: 201 | | |
| adv9-B3 | CATYTCCAACGACCTVGC | hAdv LAMP primer |
| SEQ ID NO: 202 | | |
| adv9-FIP | AGGCCGCSGTCAAYGACACGGCCCG | hAdv LAMP primer |
| SEQ ID NO: 203 | GTGAGCTTGA | |
| adv9-BIP | CCTGCACGTCTCCYGAGTTGTCGGAC | hAdv LAMP primer |
| SEQ ID NO: 204 | GCGGAGAYCTCCAG | |
| adv9-LF | ATTCTGTCGAACTCTCTTTC | hAdv LAMP primer |
| SEQ ID NO: 205 | | |
| adv9-LB | GGCCATGAACTGCTCGATCTCT | hAdv LAMP primer |
| SEQ ID NO: 206 | | |
| adv10-F3 | CSGCBAGGTCGTTGGA | hAdv LAMP primer |
| SEQ ID NO: 207 | | |
| adv10-B3 | ACCACCCTYAACTACCTCTT | hAdv LAMP primer |
| SEQ ID NO: 208 | | |
| adv10-FIP | CAGCCGMGTCTGGAACGARATGCGS | hAdv LAMP primer |
| SEQ ID NO: 209 | GCCATGAGC | |
| adv10-BIP | CGCATGACCACCTGCGCGATGCGMAA | hAdv LAMP primer |
| SEQ ID NO: 210 | CTACGCCGTCT | |
| adv10-LF | MGGMCTCAACGCCTTCTCGC | hAdv LAMP primer |
| SEQ ID NO: 211 | | |
| adv10-LB | GRTTGAGCTCCACGTGCCG | hAdv LAMP primer |
| SEQ ID NO: 212 | | |
| adv11-F3 | GCGTTGAGKCCKCCCT | hAdv LAMP primer |
| SEQ ID NO: 213 | | |
| adv11-B3 | GCGVCGCTGGGTYATGTACT | hAdv LAMP primer |
| SEQ ID NO: 214 | | |
| adv11-FIP | TCGCGCAGGTGGTCATGCGGTTCCAG | hAdv LAMP primer |
| SEQ ID NO: 215 | ACKCGGCTGT | |
| adv11-BIP | AGACGGCGTAGTTKCGCAGGTCTTCG | hAdv LAMP primer |
| SEQ ID NO: 216 | TGGCMGARCACAC | |
| adv11-LF | CSARGGGGGCGTGGTCT | hAdv LAMP primer |
| SEQ ID NO: 217 | | |
| adv11-LB | GGTAGTTRAGGGTGGTGGCG | hAdv LAMP primer |
| SEQ ID NO: 218 | | |
| adv12-F3 | SGCGAAGACGGCGTAGT | hAdv LAMP primer |
| SEQ ID NO: 219 | | |
| adv12-B3 | TGTCGGCGCGCAACTC | hAdv LAMP primer |
| SEQ ID NO: 220 | | |
| adv12-FIP | TGCGVCGCTGGGTYATGTACTTCGCA | hAdv LAMP primer |
| SEQ ID NO: 221 | GGCGCTGRAAGAGG | |
| adv12-BIP | TTCGTTGATRTCCCCCAAGGCCTTCAA | hAdv LAMP primer |
| SEQ ID NO: 222 | CTTCGCCGTGGACT | |
| adv12-LF | ACCGCCACCACCCTYAACTA | hAdv LAMP primer |
| SEQ ID NO: 223 | | |
| adv12-LB | CGCTCCATGGCCTCGTA | hAdv LAMP primer |
| SEQ ID NO: 224 | | |
| adv13-F3 | YCTGGARGCGGTGGTSC | hAdv LAMP primer |
| SEQ ID NO: 225 | | |
| adv13-B3 | CCGGTCCARGTTGGTCTG | hAdv LAMP primer |
| SEQ ID NO: 226 | | |
| adv13-FIP | GGCCCTGTTYTCGGCCAGCGCKCRAA | hAdv LAMP primer |
| SEQ ID NO: 227 | CCCCACGCA | |
| adv13-BIP | SGAYGAGGCCGGSCTGGTSTACACGT | hAdv LAMP primer |
| SEQ ID NO: 228 | TGCYGCTGTTGT | |
| adv13-LF | TSGCCAGCACCTTCTCG | hAdv LAMP primer |
| SEQ ID NO: 229 | | |
| adv13-LB | GCTGCTKCAGCGCGTGGC | hAdv LAMP primer |
| SEQ ID NO: 230 | | |
| adv14-F3 | ATCCGSCCSGAYGAGG | hAdv LAMP primer |
| SEQ ID NO: 231 | | |
| adv14-B3 | TGGTGTAGTCCTCCTGYCC | hAdv LAMP primer |
| SEQ ID NO: 232 | | |
| adv14-FIP | CCGGTCCARGTTGGTCTGCACGTSTA | hAdv LAMP primer |
| SEQ ID NO: 233 | CGACGCGCTGCTK | |
| adv14-BIP | AGGGCAACCTGGGCTCCATTGGCSGG | hAdv LAMP primer |
| SEQ ID NO: 234 | CTGYGTRCTC | |
| adv14-LF | GTTGTARCGRGCCACGCG | hAdv LAMP primer |
| SEQ ID NO: 235 | | |
| adv14-LB | TGGTKGCRCTRAACGCCTTCCT | hAdv LAMP primer |
| SEQ ID NO: 236 | | |
| adv15-F3 | GGTKGCRCTRAACGCCTTCC | hAdv LAMP primer |
| SEQ ID NO: 237 | | |
| adv15-B3 | CCTGGCTCAGGTTYACSGT | hAdv LAMP primer |
| SEQ ID NO: 238 | | |
| adv15-FIP | GCAGYGCGCTCACAAAGTTGGTTGAG | hAdv LAMP primer |
| SEQ ID NO: 239 | YACRCAGCCSGCC | |
| adv15-BIP | RATGGTGACYGAGACMCCSCARTGCA | hAdv LAMP primer |
| SEQ ID NO: 240 | GGCCYTGTCTRCTGG | |
| adv15-LF | CTGYCCCCGCGGCACGTT | hAdv LAMP primer |
| SEQ ID NO: 241 | | |
| adv15-LB | ACCAGTCSGGGCCRGACTAYTT | hAdv LAMP primer |
| SEQ ID NO: 242 | | |
| adv16-F3 | CCAACTCGCGCCTGYTG | hAdv LAMP primer |
| SEQ ID NO: 243 | | |
| adv16-B3 | GTTGGTCAGCAGGTAGTTYA | hAdv LAMP primer |
| SEQ ID NO: 244 | | |
| adv16-FIP | TCKCGGTACAGKGTCAGCARGTTGCT | hAdv LAMP primer |
| SEQ ID NO: 245 | GCTRATMGCGCCSTT | |
| adv16-BIP | GCCATMGGKCAGGCGCAKGTGTGTCC | hAdv LAMP primer |
| SEQ ID NO: 246 | TCCTGYCCCA | |
| adv16-LF | AKGTGTCCCGGGACACGCT | hAdv LAMP primer |
| SEQ ID NO: 247 | | |
| adv16-LB | GGACGAGCAYACYTTCCAGGAGAT | hAdv LAMP primer |
| SEQ ID NO: 248 | | |
| Cas12b-crRNA- | gaaatTAATACGACTCACTATAGGGTCT | Cas12b-crRNA |
| universal | AGAGGACAGAATTTTTCAACGGGTGT | scaffold template |
| scaffold | GCCAATGGCCACTTTCCAGGTGGCAA | |
| SEQ ID NO: 249 | AGCCCGTTGAGCTTCTCAAA | |
| HSV-L15P1- | ACCACCACGGGGTYSACGTGGTGCCA | HSV crRNA |
| crRNA-R | CTTCTCAGATTTGAGAAGCTCAACGG | template |
| SEQ ID NO: 250 | GCT | |
| HSV-L15P2- | CACCACCACGGGGTYSACGTGTGCCA | HSV crRNA |
| crRNA-R | CTTCTCAGATTTGAGAAGCTCAACGG | template |
| SEQ ID NO: 251 | GCT | |
| HSV-L15P3- | GGGTACAGGCTGGCAAAGTCGTGCCA | HSV crRNA |
| crRNA-R | CTTCTCAGATTTGAGAAGCTCAACGG | template |
| SEQ ID NO: 252 | GCT | |
| HSV-L15P4- | ATGCTGGGGTACAGGCTGGCGTGCCA | HSV crRNA |
| crRNA-R | CTTCTCAGATTTGAGAAGCTCAACGG | template |
| SEQ ID NO: 253 | GCT | |
| HSV-L15P5- | GATGCTGGGGTACAGGCTGGGTGCCA | HSV crRNA |
| crRNA-R | CTTCTCAGATTTGAGAAGCTCAACGG | template |
| SEQ ID NO: 254 | GCT | |
| HSV-L15P6- | CCCAGCATCATCCAGGCCCAGTGCCA | HSV crRNA |
| crRNA-R | CTTCTCAGATTTGAGAAGCTCAACGG | template |
| SEQ ID NO: 255 | GCT | |
| HSV-L15P7- | GSCCKCAGGGAGAGCGTRCTGTGCCA | HSV crRNA |
| crRNA-R | CTTCTCAGATTTGAGAAGCTCAACGG | template |
| SEQ ID NO: 256 | GCT | |
| HSV-L15P8- | GGSCGACGGCTGTTCTTCGTGTGCCA | HSV crRNA |
| crRNA-R | CTTCTCAGATTTGAGAAGCTCAACGG | template |
| SEQ ID NO: 257 | GCT | |
| adv-L12P1- | TACCTCTTTCAGCGCCTGCGGTGCCA | hAdv crRNA |
| crRNA-R | CTTCTCAGATTTGAGAAGCTCAACGG | template |
| SEQ ID NO: 258 | GCT | |

| | | |
|---|---|---|
| adv-L12P2- | GAACACACCGCCACCACCCTGTGCCA | hAdv crRNA |
| crRNA-R | CTTCTCAGATTTGAGAAGCTCAACGG | template |
| SEQ ID NO: 259 | GCT | |
| adv-L12P3- | TTATGTACTTCTTCGTGGCAGTGCCAC | hAdv crRNA |
| crRNA-R | TTCTCAGATTTGAGAAGCTCAACGGG | template |
| SEQ ID NO: 260 | CT | |
| adv-L12P4- | GTGGCGGTGTGTTCTGCCACGTGCCA | hAdv crRNA |
| crRNA-R | CTTCTCAGATTTGAGAAGCTCAACGG | template |
| SEQ ID NO: 261 | GCT | |
| adv-L12P5- | GCGGTGTGTTCTGCCACGAAGTGCCA | hAdv crRNA |
| crRNA-R | CTTCTCAGATTTGAGAAGCTCAACGG | template |
| SEQ ID NO: 262 | GCT | |
| adv-L12P6- | TTCTGCCACGAAGAAGTACAGTGCCA | hAdv crRNA |
| crRNA-R | CTTCTCAGATTTGAGAAGCTCAACGG | template |
| SEQ ID NO: 263 | GCT | |
| adv-L12P7- | AAGTACATAACCCAGCGYCGGTGCCA | hAdv crRNA |
| crRNA-R | CTTCTCAGATTTGAGAAGCTCAACGG | template |
| SEQ ID NO: 264 | GCT | |
| adv-L12P8- | AGGCCTTGGGGGATATCAACGTGCCA | hAdv crRNA |
| crRNA-R | CTTCTCAGATTTGAGAAGCTCAACGG | template |
| SEQ ID NO: 265 | GCT | |
| adv-L12P9- | CTTGAGGCCTTGGGGGATATGTGCCA | hAdv crRNA |
| crRNA-R | CTTCTCAGATTTGAGAAGCTCAACGG | template |
| SEQ ID NO: 266 | GCT | |
| adv-L12P10- | GTGGATTCGTTGATATCCCCGTGCCA | hAdv crRNA |
| crRNA-R | CTTCTCAGATTTGAGAAGCTCAACGG | template |
| SEQ ID NO: 267 | GCT | |
| adv-L12P11- | GTTGATATCCCCCAAGGCCTGTGCCA | hAdv crRNA |
| crRNA-R | CTTCTCAGATTTGAGAAGCTCAACGG | template |
| SEQ ID NO: 268 | GCT | |
| adv-L12P12- | GCCTCGTAGAAGTCCACGGCGTGCCA | hAdv crRNA |
| crRNA-R | CTTCTCAGATTTGAGAAGCTCAACGG | template |
| SEQ ID NO: 269 | GCT | |
| adv-L12P13- | GCGCGCAACTCCCAGTTTTTGTGCCA | hAdv crRNA |
| crRNA-R | CTTCTCAGATTTGAGAAGCTCAACGG | template |
| SEQ ID NO: 270 | GCT | |
| adv-L12P14- | | hAdv crRNA |
| crRNA-R | TAGAAGTCCACGGCGAAGTTGTGCCA | template |
| SEQ ID NO: 271 | CTTCTCAGATTTGAGAAGCTCAACGG | |
| | GCT | |
| adv-L12P15- | AGAAGTCCACGGCGAAGTTGGTGCCA | hAdv crRNA |
| crRNA-R | CTTCTCAGATTTGAGAAGCTCAACGG | template |
| SEQ ID NO: 272 | GCT | |
| adv-L12P16- | GAAGTCCACGGCGAAGTTGAGTGCCA | hAdv crRNA |
| crRNA-R | CTTCTCAGATTTGAGAAGCTCAACGG | template |
| SEQ ID NO: 273 | GCT | |
| adv-L12P17- | AAGTCCACGGCGAAGTTGAAGTGCCA | hAdv crRNA |
| crRNA-R | CTTCTCAGATTTGAGAAGCTCAACGG | template |
| SEQ ID NO: 274 | GCT | |

### Methods:

### Virus Culture:

Human adenovirus 1 (ATCC VR-1) was propagated using A549 human lung epithelial cell line (ATCC CCL-185) grown in Ham's F-12K (Kaighn's) Medium (Thermo Fisher Scientific). Viruses were concentrated and purified using a chromatography-based Adeno-X Maxi Purification system (Takara Bio) according to the manufacturer's instructions. Herpes simplex virus 1 (ATCC VR-260) was propagated using Vero cell line (ATCC CCL-81) grown in Dulbecco's Modified Eagle Medium (Thermo Fisher Scientific). Viruses were concentrated by ultracentrifugation and purified using iodixanol density gradient ultracentrifugation.

### Virus DNA Extraction:

DNA was extracted using the QIAamp DNA Mini Kit (QIAGEN) according to the manufacturer's protocol.

### Bulk RT-LAMP-Cas12b Reactions:

The DNA/RNA target samples were mixed with 1.6 μM FIP primers, 1.6 μM BIP primers, 0.2 μM F3 primers, 0.2 μM B3 primers, 0.4 μM LoopF primers, 0.4 μM LoopB primers, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, and 50 nM crRNA in 1× isothermal amplification buffer, unless otherwise indicated. To detect the LAMP signal, 250 mM SYTO-82 fluorescent nucleic acid stain was added to the reaction. The reaction mixture was incubated at 60° C. and fluorescence kinetics were monitored for 1-2 h using Roche Light Cycler 96. The fluorescence values displayed in the results were the fluorescence levels determined by Roche Light Cycler 96 software. To mimic the complexity of real samples, 1 ng/μL human genomic DNA was also added to the reactions.

### RADICA Reactions by QIAcuity Digital Nanoplate:

The RADICA reaction was prepared by adding 250 nM Cyanine 680 succinimidyl ester (biotium) as reference dye to the RT-LAMP-Cas12b bulk reactions stated above and partitioned on QIAcuity digital nanoplates (˜0.91 nL partition volume, 26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

Results:

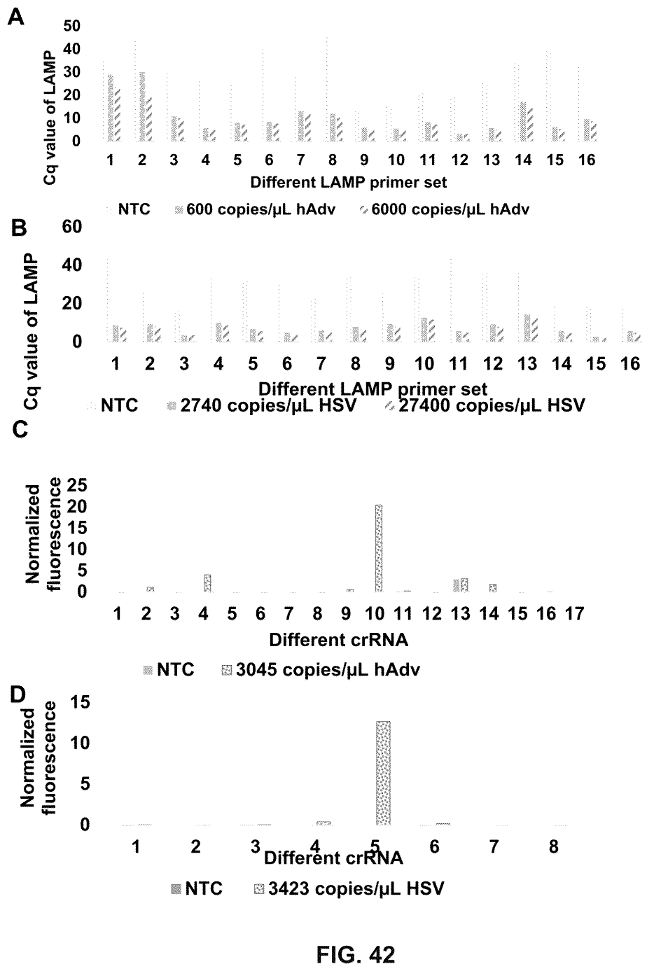

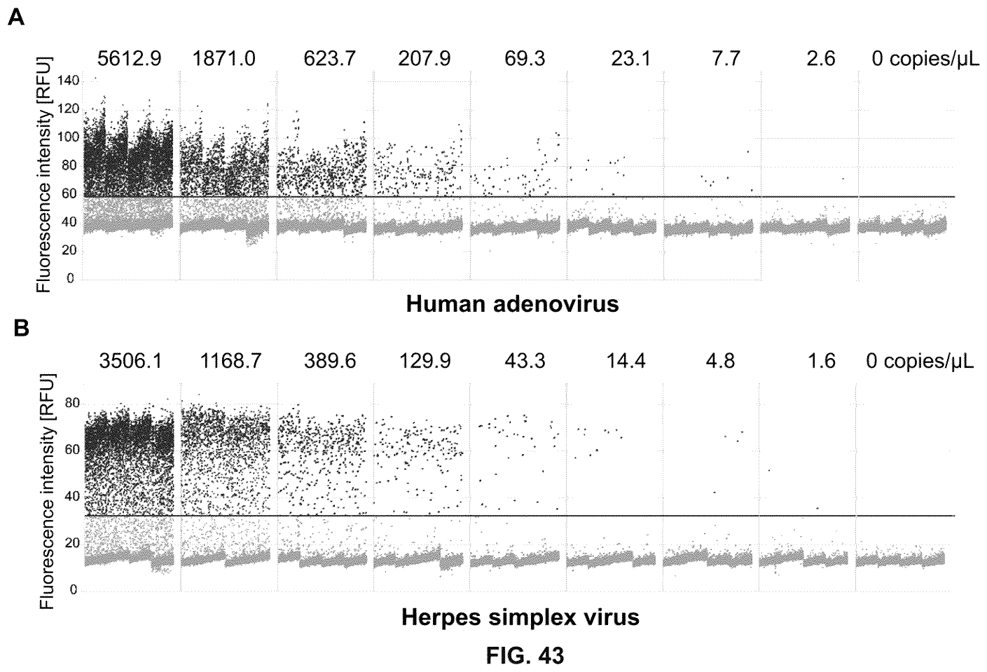

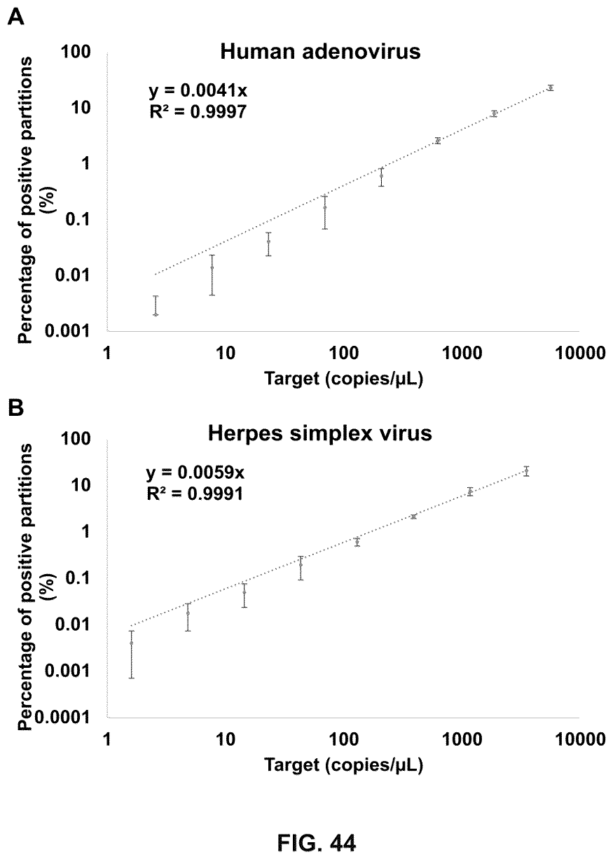

To make use of the speed and quantification ability of RADICA, we have designed RADICA assays to detect and quantify the most common viral contaminants in biomanufacture: human adenovirus and herpes simplex virus [Barone, P. W. et al. Nature biotechnology 38: 563-572 (2020)]. Different LAMP primer sets and crRNA were designed and screened by bulk LAMP-Cas12b reaction, and the combination of LAMP primer set and crRNA with the highest speed were selected for the following RADICA experiment (FIG. 42). DNA extracted from human adenovirus and herpes simplex virus were serial diluted to across a dynamic range of over 4 orders of magnitude (human adenovirus: 2.6 to 5612 copies/μL; herpes simplex virus: 1.6 to 3506 copies/μL) and tested by the respective RADICA assay. As we expected, the ratio of positive partitions increased with the increase of target DNA for both virus targets (FIG. 43). The positive partition percentage showed a high linear relationship with the input target concentration (both R² values>0.99) (FIG. 44), indicating the ability of RADICA to quantify real virus sample within one hour. The rapid speed and good quantification result of RADICA on human adenovirus and herpes simplex virus demonstrated the wide applications of RADICA in different areas.

### Example 17

In clinical settings, to confirm the success of sampling as well as the reaction, there is a need to test the presence of human control as well as the intended target in a same reaction. Thus, a multiplex assay is needed to test both the target and the human control. One strategy of multiplex RADICA is use different Cas effectors with different specificity on the reporter [Gootenberg, J. S. et al. Science 360: 439-444 (2018)], another strategy is couple RADICA with other probe-based isothermal amplification methods. Here we validated the multiplex RADICA method using the second strategy, with primers and crRNA shown in Table 9.

TABLE 9
Primers, crRNA for multiplex RADICA (digital RT-LAMP-Cas12b+ probe-based
LAMP) targeting SARS-CoV-2 and human control.

| Name | Sequence | Application |
|---|---|---|
| FQ5T | /56-FAM/TTTTT/3IABKFQ/ | FQ reporter |
| N2-LAMP- | GUCUAGAGGACAGAAUUUUUCAACGGG | SARS-CoV-2 |
| crRNA11 | UGUGCCAAUGGCCACUUUCCAGGUGG | crRNA for |
| SEQ ID NO: | CAAAGCCCGUUGAGCUUCUCAAAUCUG | Cas12b* |
| 50 | AGAAGUGGCACCGAAGAACGCUGAAGC | |
| | GCUG | |
| N2-WSLAMP- | GCTGCTGAGGCTTCTAAG | SARS-CoV-2 |
| F3 | | LAMP primers* |
| SEQ ID NO: | | |
| 51 | | |
| N2-WSLAMP- | GCGTCAATATGCTTATTCAGC | SARS-CoV-2 |
| B3 | | LAMP primers* |
| SEQ ID NO: | | |
| 52 | | |
| N2-WSLAMP- | GCGGCCAATGTTTGTAATCAGTAGACGT | SARS-CoV-2 |
| FIP | GGTCCAGAACAA | LAMP primers* |
| SEQ ID NO: | | |
| 53 | | |
| N2-WSLAMP- | TCAGCGTTCTTCGGAATGTCGCTGTGTA | SARS-CoV-2 |
| BIP | GGTCAACCACG | LAMP primers* |
| SEQ ID NO: | | |
| 54 | | |
| N2-WSLAMP- | CCTTGTCTGATTAGTTCCTGGT | SARS-CoV-2 |
| LoopF | | LAMP primers* |
| SEQ ID NO: | | |
| 55 | | |
| N2-WSLAMP- | TGGCATGGAAGTCACACC | SARS-CoV-2 |
| LoopB | | LAMP primers* |
| SEQ ID NO: | | |
| 56 | | |
| ACTB-F3 | AGTACCCCATCGAGCACG | human ACTB |
| SEQ ID NO: | | LAMP primer^{∧} |
| 275 | | |
| ACTB-B3 | AGCCTGGATAGCAACGTACA | human ACTB |
| SEQ ID NO: | | LAMP primer^{∧} |
| 276 | | |
| ACTB-FIP | GAGCCACACGCAGCTCATTGTATCACCA | human ACTB |
| SEQ ID NO: | ACTGGGACGACA | LAMP primer^{∧} |
| 277 | | |
| ACTB-BIP | CTGAACCCCAAGGCCAACCGGCTGGGG | human ACTB |
| SEQ ID NO: | TGTTGAAGGTC | LAMP primer^{∧} |
| 278 | | |
| ACTB-LF | TGTGGTGCCAGATTTTCTCCA | human ACTB |
| SEQ ID NO: | | LAMP primer^{∧} |
| 279 | | |
| ACTB-LB | CGAGAAGATGACCCAGATCATGT | human ACTB |
| SEQ ID NO: | | LAMP primer^{∧} |
| 280 | | |
| ACTB-FIP- | /56- | human ACTB |
| ROX | ROXN/GAGCCACACGCAGCTCATTGTAT | LAMP probe |
| SEQ ID NO: | CACCAACTGGGACGACA | |
| 281 | | |
| ACTB-F1-RQ | TACAATGAGCTGCGTGTGGCTC/3IAbRQ | human ACTB |
| SEQ ID NO: | Sp/ | LAMP probe |
| 282 | | |

*Joung, J. et al., New England Journal of Medicine (2020).
^{∧}Zhang, Y. et al. Biotechniques 69: 178-185 (2020)

### Methods:

### Multiplex RADICA to Detection SARS-CoV-2 N Gene and Human Background:

ACTB-probe were prepared by mixing equal molar of ACTB-FIP-ROX/ACTB-F1-RQ, followed by 95° C. 5 min followed by slowly cooling down (ramp rate=1° C./s) to room temperature. The DNA/RNA target samples were mixed with 1.6 μM FIP primers (N2-WSLAMP-FIP and ACTB-FIP), 1.6 μM BIP primers (N2-WSLAMP-BIP and ACTB-BIP), 0.2 μM F3 primers (N2-WSLAMP-F3 and ACTB-F3), 0.2 μM B3 primers (N2-WSLAMP-B3 and ACTB-B3), 0.4 μM LoopF primers (N2-WSLAMP-LoopF and ACTB-LF), 0.4 μM LoopB primers (N2-WSLAMP-LoopB and ACTB-LB), 0.4 μM ACTB-FIP-ROX/ACTB-F1-RQ duplex, 1.4 mM dNTPs, 8 mM MgSO₄, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, 50 nM crRNA and 250 nM Cyanine 680 succinimidyl ester (biotium) as reference dye in 1× isothermal amplification buffer, unless otherwise indicated. The reactions were partitioned on QIAcuity digital nanoplates (˜0.91 nL partition volume, ˜26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

### Results:

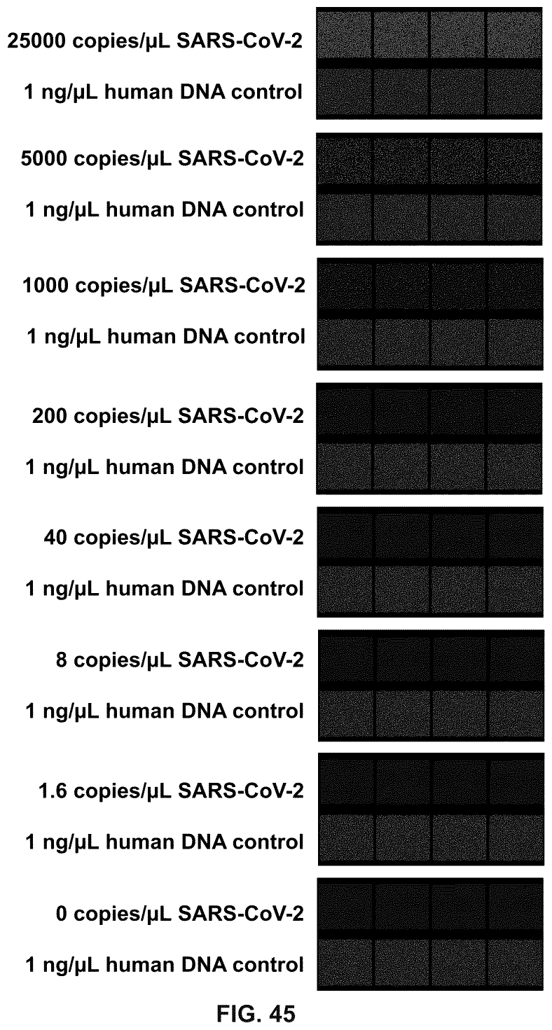

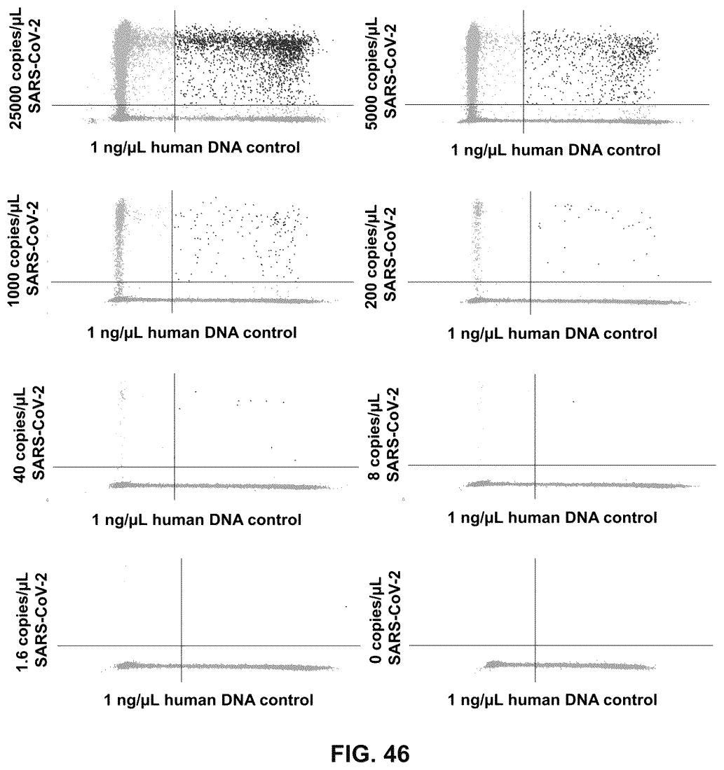

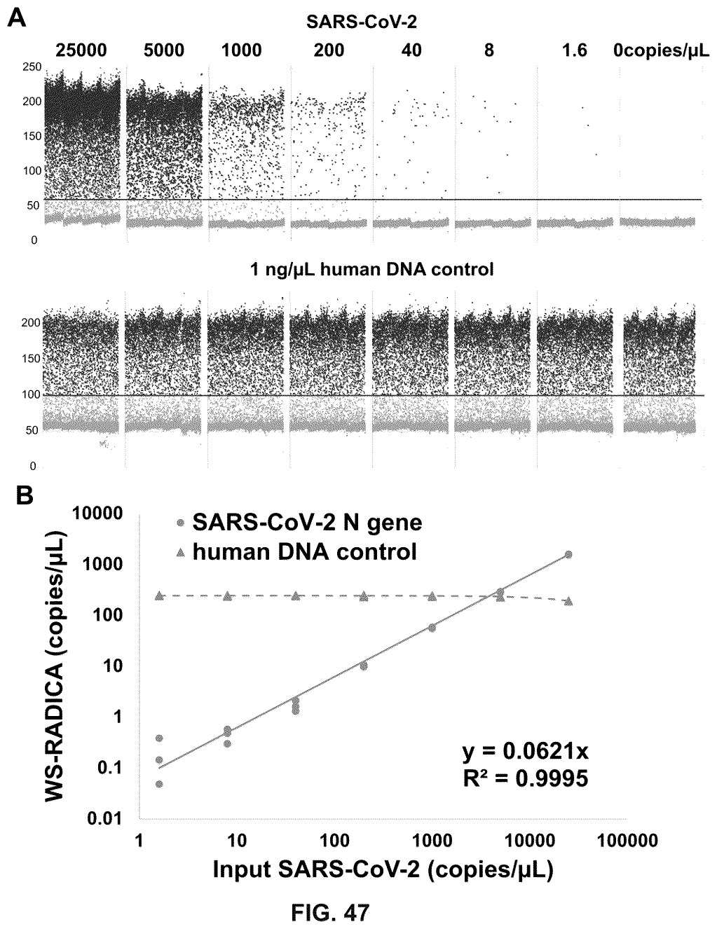

Here we validated a multiplex RADICA method. First, a multiplex method targeting two targets that combine RADICA (FAM fluorescent colour signal, targeting SARS-CoV-2 N gene) with probe-based LAMP reaction (ROX fluorescent colour signal, targeting human ACTB gene) were designed and tested [Zhang, Y. et al. Biotechniques 69: 178-185 (2020)]. SARS-CoV-2 N gene were detected by N gene-specific LAMP primer and crRNA, human ACTB gene were detected by ACTB gene-specific LAMP primer and probe. Different concentrations of SARS-CoV-2 RNA in a constant amount of human background were tested using this method. From the result, the two detection targets are compatible and do not interfere with each other in the same multiplex reaction. With the decrease of SARS-CoV-2 RNA (from 25000 to 0 copies/μL) in a constant human DNA (1 ng/μL), the result showed decreasing FAM-positive partitions and constant ROX-positive partitions in both the position plot and scatter plot (FIGS. 45 and 46). Also, the measured RADICA result on N gene showed a high linear relationship with the input SARS-CoV-2 RNA copy number (R²>0.99) (FIG. 47). The results above prove that RADICA can be used for multiplex detection and quantification.

### Example 18

### Multiplex RADICA (Digital RT-LAMP-Cas12b) in Detecting SARS-CoV-2 Wildtype and Mutant

As there is an emerging number of SARS-CoV-2 mutants with high transmission and infection activity, we tested whether multiplex RADICA could be used to detect both the wildtype and mutant using primers and crRNA shown in Table 10.

TABLE 10
Primers, crRNA for multiplex RADICA (digital RT-LAMP-Cas12b+ probe-
based LAMP) targeting SARS-CoV-2 wildtype and mutants.

| Name | Sequence | Application |
|---|---|---|
| FQ5T | /56-FAM/TTTTT/3IABKFQ/ | FQ reporter |
| N2-LAMP- | GUCUAGAGGACAGAAUUUUUCAACGGG | SARS-CoV-2 |
| crRNA11 | UGUGCCAAUGGCCACUUUCCAGGUGG | crRNA for |
| SEQ ID NO: | CAAAGCCCGUUGAGCUUCUCAAAUCUG | Cas12b* |
| 50 | AGAAGUGGCACCGAAGAACGCUGAAGC | |
| | GCUG | |
| N2-WSLAMP- | GCTGCTGAGGCTTCTAAG | SARS-CoV-2 |
| F3 | | LAMP primers* |
| SEQ ID NO: | | |
| 51 | | |
| N2-WSLAMP- | GCGTCAATATGCTTATTCAGC | SARS-CoV-2 |
| B3 | | LAMP primers* |
| SEQ ID NO: | | |
| 52 | | |
| N2-WSLAMP- | GCGGCCAATGTTTGTAATCAGTAGACGT | SARS-CoV-2 |
| FIP | GGTCCAGAACAA | LAMP primers* |
| SEQ ID NO: | | |
| 53 | | |
| N2-WSLAMP- | TCAGCGTTCTTCGGAATGTCGCTGTGTA | SARS-CoV-2 |
| BIP | GGTCAACCACG | LAMP primers* |
| SEQ ID NO: | | |
| 54 | | |
| N2-WSLAMP- | CCTTGTCTGATTAGTTCCTGGT | SARS-CoV-2 |
| LoopF | | LAMP primers* |
| SEQ ID NO: | | |
| 55 | | |
| N2-WSLAMP- | TGGCATGGAAGTCACACC | SARS-CoV-2 |
| LoopB | | LAMP primers* |
| SEQ ID NO: | | |
| 56 | | |
| S1-F3 | TCTTACCTTTCTTTTCCAATGT | SARS-CoV-2 S |
| SEQ ID NO: | | LAMP primer |
| 283 | | |
| S1-LF | AAATGGTAGGACAGGGTTA | SARS-CoV-2 S |
| SEQ ID NO: | | LAMP primer |
| 284 | | |
| S1-LB | AAGTCTGTGAATTTCAATTTTGTAA | SARS-CoV-2 S |
| SEQ ID NO: | | LAMP primer |
| 285 | | |
| S1-B3 | GAGAGACATATTCAAAAGTGC | SARS-CoV-2 S |
| SEQ ID NO: | | LAMP primer |
| 286 | | |
| S1-F1P-WT | TGGAAGCAAAATAAACACCATCATT | SARS-CoV-2 S |
| SEQ ID NO: | TCTCTGGGACCAATGKTAYT | LAMP primer |
| 287 | | |
| S1-B1P-WT | ATTGTTAATAACGCTACTAATGTTGTTAT | SARS-CoV-2 S |
| SEQ ID NO: | CAACTTTTGTTGTTTTTGTGG | LAMP primer |
| 288 | | |
| S1-F1P-alpha- | /5Cy5/ | SARS-CoV-2 |
| CY5-4bp | TGGAAGCAAAATAAACACCATCATT | alpha mutant S |
| SEQ ID NO: | TACTTGGTTCCATGCTATCTC | LAMP probe |
| 289 | | |
| S1-F1P-beta- | /5TEX615/TGGAAGCAAAATAAACACCAT | SARS-CoV-2 beta |
| TEX615- | CATT CAATGGTACTAAGAGGTTTCC | mutant S LAMP |
| 1 mismatch | | probe |
| SEQ ID NO: | | |
| 290 | | |
| S1-F1C-RQ | AATGATGGTGTTTATTTTGCTTCCA/ | SARS-CoV-2 S |
| SEQ ID NO: | 3IAbRQSp/ | LAMP probe |
| 291 | | |

*Joung, J. et al., New England Journal of Medicine (2020).

### Methods:

### Multiplex RADICA to Detection SARS-CoV-2 Wildtype and Mutant:

S gene-probe were prepared by mixing equal molar of S1-F1P-alpha-CY5-4 bp/S1-F1C-RQ or S1-F1P-beta-TEX615-1mismatch/S1-F1C-RQ, followed by 95° C. 5 min followed by slowly cooling down (ramp rate=1° C./s) to room temperature. The RNA target samples were mixed with 1.6 μM FIP primers (N2-WSLAMP-FIP and S1-FIP-WT), 1.6 μM BIP primers (N2-WSLAMP-BIP and S1-BIP-WT), 0.2 μM F3 primers (N2-WSLAMP-F3 and S1-F3), 0.2 μM B3 primers (N2-WSLAMP-B3 and S1-133), 0.4 μM LoopF primers (N2-WSLAMP-LoopF and S1-LF), 0.4 μM LoopB primers (N2-WSLAMP-LoopB and S1-LB), 0.1 μM S1-F1P-alpha-CY5-4 bp/S1-F1C-RQ and 0.1 μM S1-F1P-beta-TEX615-1mismatch/S1-F1C-RQ, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, 50 nM crRNA and 250 nM Cyanine 680 succinimidyl ester (biotium) as reference dye in 1× isothermal amplification buffer, unless otherwise indicated. The reactions were partitioned on QIAcuity digital nanoplates (˜0.91 nL partition volume, 26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal to noise ratio, exposure duration (200 ms to 800 ms) and gain (2 to 8) were adjusted for each experiment. Usually, a 600 ms/6 will be used for the sample reading. Positive partition percentages, as well as nucleic acid concentration, were calculated by QIAcuity software.

### Results:

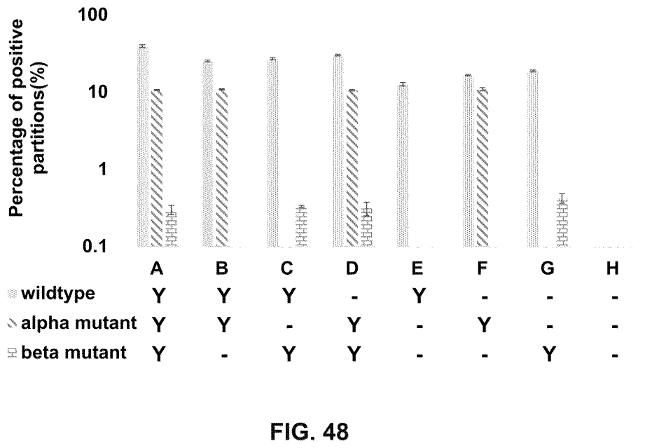

For multiplex detection of wildtype and mutant in one reaction, RADICA targeting N gene of SARS-CoV-2 (FAM fluorescent colour signal, could detect both wildtype, alpha and beta mutant) and probe-based digital RT-LAMP targeting mutant (CY5 fluorescent colour signal specific for alpha mutant detection and ROX fluorescent colour signal specific for beta mutant detection) were tested on different SARS-CoV-2 wildtype and mutant. N gene LAMP primer/crRNA were used to detect SARS-CoV-2 both wildtype and mutant. For the mutant-specific primer design, LAMP primers and probes specifically targeting SARS-CoV-2 mutant but not wildtype were designed by the principle of allele-specific LAMP [Gill, P. and Hadian Amree, A. Avicenna J Med Biotechnol 12: 2-8 (2020)]. The multiplexed assays were validated using a mixture of synthetic RNA of SARS-CoV-2 wildtype, alpha and beta mutant. From the result (FIG. 48), the either wildtype or alpha/beta mutant in the sample lead to a high FAM-positive partition ratio, meaning that N gene primer/crRNA we use is able to detect both SARS-Co-V wildtype and mutant. For the mutant-specific result, CY5-positive partitions only show up if there's alpha mutant in the sample and ROX-positive partitions only show up if there's beta mutant in the sample, indicating the specific LAMP S gene primer/probe could specifically detect mutant of SARS-CoV-2 but not wildtype. Above all, the results above indicate multiplex RADICA could be used to specifically detect nucleic acid wildtype and mutants.

### Example 19

### Detecting Three SARS-CoV-2 Genes and One Human Gene by Multiplex RADICA in One Reaction

As there is a need to detect more than 2 targets in many applications, we tested the performance of multiplex RADICA in detecting 4 targets simultaneously. Primers, probes and crRNA (Table 11) were designed to detect SARS-CoV-2 N gene (FAM channel), SARS-CoV-2 E gene (HEX channel), SARS-CoV-2 ORF1ab gene (CY5 channel) and human ACTB gene (ROX channel) in one reaction.

TABLE 11
Primers, crRNA for multiplex RADICA (digital RT-LAMP-Cas12b+ probe-
based LAMP) targeting three SARS-CoV-2 genes and one human gene in one
reaction.

| Name | Sequence | Application |
|---|---|---|
| FQ5T | /56-FAM/TTTTT/3IABKFQ/ | FQ reporter |
| N2-LAMP- | GUCUAGAGGACAGAAUUUUUCAACGGG | SARS-CoV-2 N |
| crRNA11 | UGUGCCAAUGGCCACUUUCCAGGUGG | gene crRNA for |
| SEQ ID NO: | CAAAGCCCGUUGAGCUUCUCAAAUCUG | Cas12b* |
| 50 | AGAAGUGGCACCGAAGAACGCUGAAGC | |
| | GCUG | |
| N2-WSLAMP- | GCTGCTGAGGCTTCTAAG | SARS-CoV-2 N |
| F3 | | gene LAMP |
| SEQ ID NO: | | primers* |
| 51 | | |
| N2-WSLAMP- | GCGTCAATATGCTTATTCAGC | SARS-CoV-2 N |
| B3 | | gene LAMP |
| SEQ ID NO: | | primers* |
| 52 | | |
| N2-WSLAMP- | GCGGCCAATGTTTGTAATCAGTAGACGT | SARS-CoV-2 N |
| FIP | GGTCCAGAACAA | gene LAMP |
| SEQ ID NO: | | primers* |
| 53 | | |
| N2-WSLAMP- | TCAGCGTTCTTCGGAATGTCGCTGTGTA | SARS-CoV-2 N |
| BIP | GGTCAACCACG | gene LAMP |
| SEQ ID NO: | | primers* |
| 54 | | |
| N2-WSLAMP- | CCTTGTCTGATTAGTTCCTGGT | SARS-CoV-2 N |
| LoopF | | gene LAMP |
| SEQ ID NO: | | primers* |
| 55 | | |
| N2-WSLAMP- | TGGCATGGAAGTCACACC | SARS-CoV-2 N |
| LoopB | | gene LAMP |
| SEQ ID NO: | | primers* |
| 56 | | |
| E1-F3 | TGAGTACGAACTTATGTACTCAT | SARS-CoV-2 E |
| SEQ ID NO: | | gene LAMP |
| 292 | | primers^{∧} |
| E1-B3 SEQ ID | TTCAGATTTTTAACACGAGAGT | SARS-CoV-2 E |
| NO: 293 | | gene LAMP |
| | | primers^{∧} |
| E1-FIP SEQ | ACCACGAAAGCAAGAAAAAGAAGTTCGT | SARS-CoV-2 E |
| ID NO: 294 | TTCGGAAGAGACAG | gene LAMP |
| | | primers^{∧} |
| E1-BIP SEQ | TTGCTAGTTACACTAGCCATCCTTAGGT | SARS-CoV-2 E |
| ID NO: 295 | TTTACAAGACTCACGT | gene LAMP |
| | | primers^{∧} |
| E1-LF SEQ ID | CGCTATTAACTATTAACG | SARS-CoV-2 E |
| NO: 296 | | gene LAMP |
| | | primers^{∧} |
| E1-LB SEQ ID | GCGCTTCGATTGTGTGCGT | SARS-CoV-2 E |
| NO: 297 | | gene LAMP |
| | | primers^{∧} |
| E1-FIP-HEX | /5HEX/ACCACGAAAGCAAGAAAAAGAAG | SARS-CoV-2 E |
| SEQ ID NO: | TTCGTTTCGGAAGAGACAG | gene LAMP probe |
| 298 | | |
| E1-F1-FQ | ACTTCTTTTTCTTGCTTTCGTGGT/ | SARS-CoV-2 E |
| SEQ ID NO: | 3IABKFQ/ | gene LAMP probe |
| 299 | | |
| ACTB-F3 | AGTACCCCATCGAGCACG | human ACTB |
| SEQ ID NO: | | LAMP primer^{∧} |
| 275 | | |
| ACTB-B3 | AGCCTGGATAGCAACGTACA | human ACTB |
| SEQ ID NO: | | LAMP primer^{∧} |
| 276 | | |
| ACTB-FIP | GAGCCACACGCAGCTCATTGTATCACCA | human ACTB |
| SEQ ID NO: | ACTGGGACGACA | LAMP primer^{∧} |
| 277 | | |
| ACTB-BIP | CTGAACCCCAAGGCCAACCGGCTGGGG | human ACTB |
| SEQ ID NO: | TGTTGAAGGTC | LAMP primer^{∧} |
| 278 | | |
| ACTB-LF | TGTGGTGCCAGATTTTCTCCA | human ACTB |
| SEQ ID NO: | | LAMP primer^{∧} |
| 279 | | |
| ACTB-LB | CGAGAAGATGACCCAGATCATGT | human ACTB |
| SEQ ID NO: | | LAMP primer^{∧} |
| 280 | | |
| ACTB-FIP- | /56- | human ACTB |
| ROX | ROXN/GAGCCACACGCAGCTCATTGTAT | LAMP probe |
| SEQ ID NO: | CACCAACTGGGACGACA | |
| 281 | | |
| ACTB-F1-RQ | TACAATGAGCTGCGTGTGGCTC/ | human ACTB |
| SEQ ID NO: | 3IAbRQSp/ | LAMP probe |
| 282 | | |
| Orf1a_F3 SEQ | CGGTGGACAAATTGTCAC | SARS-CoV-2 |
| ID NO: 300 | | ORF1ab gene |
| | | LAMP primers^{∧} |
| Orf1a_B3 SEQ | CTTCTCTGGATTTAACACACTT | SARS-CoV-2 |
| ID NO: 301 | | ORF1ab gene |
| | | LAMP primers^{∧} |
| Orf1a_LF SEQ | TTACAAGCTTAAAGAATGTCTGAACACT | SARS-CoV-2 |
| ID NO: 302 | | ORF1ab gene |
| | | LAMP primers^{∧} |
| Orf1a_LB SEQ | TTGAATTTAGGTGAAACATTTGTCACG | SARS-CoV-2 |
| ID NO: 303 | | ORF1ab gene |
| | | LAMP primers^{∧} |
| Orf1a_FIP | TCAGCACACAAAGCCAAAAATTTATCTG | SARS-CoV-2 |
| SEQ ID NO: | TGCAAAGGAAATTAAGGAG | ORF1ab gene |
| 304 | | LAMP primers^{∧} |
| Orf1a_BIP | TATTGGTGGAGCTAAACTTAAAGCCCTG | SARS-CoV-2 |
| SEQ ID NO: | TACAATCCCTTTGAGTG | ORF1ab gene |
| 305 | | LAMP primers^{∧} |
| Orf1a_FIP- | /5Cy5/ | SARS-CoV-2 |
| CY5 SEQ ID | TCAGCACACAAAGCCAAAAATTTAT | ORF1ab gene |
| NO: 306 | CTGTGCAAAGGAAATTAAGGAG | LAMP probe |
| Orf1a_F1C- | ATAAATTTTTGGCTTTGTGTGCTGA/3IAb | SARS-CoV-2 |
| RQ SEQ ID | RQSp/ | ORF1ab gene |
| NO: 307 | | LAMP probe |

*Joung, J. et al., New England Journal of Medicine (2020).
^{∧}Zhang, Y. et al. Biotechniques 69: 178-185 (2020)

### Methods:

### Multiplex RADICA to Detect Three SARS-CoV-2 Genes and Human Background:

LAMP probe set were prepared by mixing equal molar of E1-HP-HEX/E1-F1-1-PQ, or ACTB-FIP-ROX/ACTB-F1-RQ or Orf1a_FIP-CY5/001a_F1C-RQ, followed by 95° C. 5 min followed by slowly cooling down (ramp rate=1° C./s) to room temperature. 10×LAMP primer sets were prepared as follows: 1.6 μM FIP primers, 1.6 μM BIP primers, 0.2 μM F3 primers, μM B3 primers, 0.4 μM LoopF primers, 0.4 μM LoopB primers were premixed for each gene. The RNA target samples were mixed with 1×N LAMP primer set, 0.5×E LAMP primer set, 0.5×ACTB LAMP primer set, 0.5×ORF1ab LAMP primer set, 0.3 μM E1-FIP-HEX/E1-F1-FQ duplex, 0.1 μM ACTB-FIP-ROX/ACTB-F1-RQ duplex, 0.2 μMOrf1a_FIP-CY5/Orf1a_F1C-RQ duplex, 1.4 mM dNTPs, 8 mM MgSO4, 2 μM FQ-5T reporters, 0.96 U/μL Bst 2.0 WarmStart polymerase, 0.3 U/μL WarmStart RTx Reverse Transcriptase (for RNA), 1 U/μL RNase Inhibitor, 50 mM Taurine, 50 nM Cas12b, 50 nM crRNA and 250 nM Cyanine 680 succinimidyl ester (biotium) as reference dye in 1× isothermal amplification buffer, unless otherwise indicated. The reactions were partitioned on QIAcuity digital nanoplates (˜0.91 nL partition volume, ˜26,000 partitions per reaction). 40 μL reaction mixtures were loaded onto the QIAcuity digital nanoplate and into the QIAcuity Digital PCR System. In the QIAcuity machine, the reactions were automatically partitioned into 26,000 microwells followed by 60° C. incubation for 1 hour and end-point fluorescence detection. To get the best signal-to-noise ratio, exposure duration (200 ms to 800 ms) and gain (2 to 8) were adjusted for each experiment. Usually, a 600 ms/2 will be used for the sample reading. Positive partition percentages, as well as nucleic acid concentration, were calculated by QIAcuity software.

### Results:

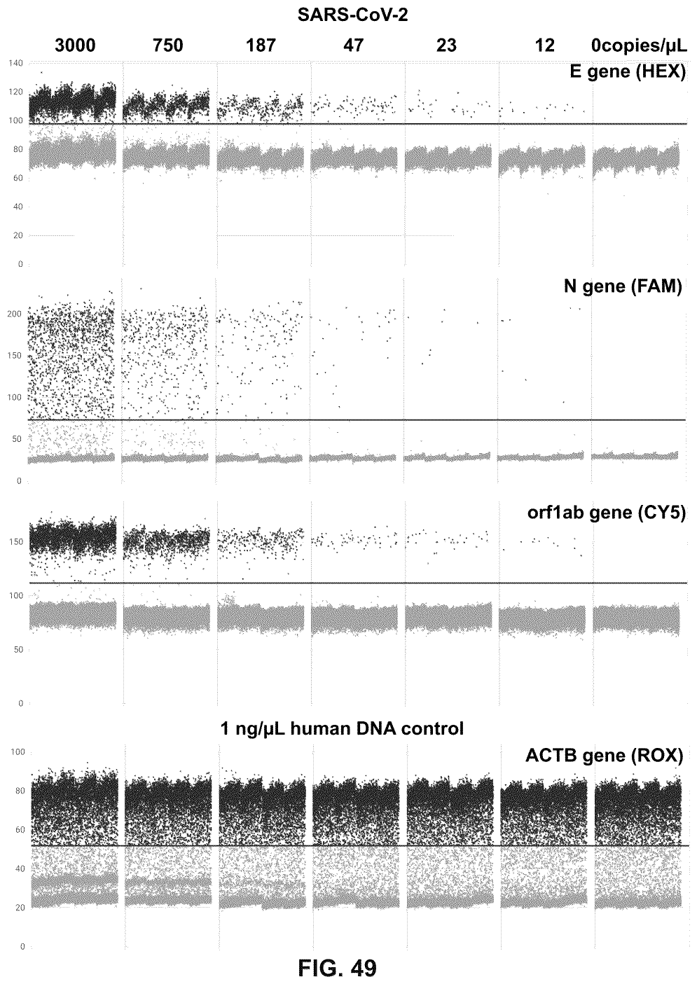

Serial dilutions of SARS-CoV-2 RNA in a constant human genomic DNA background were used to validate this multiplex reaction. From the scatter plot (FIG. 49), we can see the positive partitions for three SARS-CoV-2 genes decrease when less SARS-CoV-2 RNA was present in the sample, indicating multiplex RADICA is able to quantify the targets. Since the human DNA concentration was constant in all the samples (1 ng/μL), we can see similar levels of human ACTB gene-positive partitions in all the samples. And when we compare the relationship of the input RNA concentration and the measured concentration by RADICA, good linear relationships were shown in all the three SARS-CoV-2 targets, demonstrating the good performance of RADICA in multiplex detection and quantification.

### SUMMARY

RADICA can be easily extended to a variety of clinical, research, environmental, and biomanufacturing applications, such as liquid biopsy, rare mutation detection, gene expression analysis, gene editing detection, sequencing library quantification, environmental monitoring, cancer research, and cell therapies. RADICA offers a customizable solution that is amenable to many DNA isothermal amplification platforms such as recombinase polymerase amplification (RPA) [Piepenburg, O. et al. PLOS Biology 4: e204 (2006)], loop-mediated isothermal amplification (LAMP) [Notomi, T. et al. Nucleic acids research 28: E63-E63 (2000)], rolling circle amplification (RCA) [Lizardi, P. M. et al. Nature Genetics 19: 225-232 (1998)], strand displacement amplification (SDA) [Walker, G. T. et al. Nucleic acids research 20: 1691-1696 (1992)], or other isothermal amplifications, as well as the use of other Cas proteins, such as Cas13a, Cas12b, Cas14 for multiplex detection [Gootenberg, J. S. et al. Science 360: 439-444 (2018)]. Also, RADICA could be used on different microfluidic or droplet-based partition devices such as Clarity digital PCR system (JNMedsys), QuantStudio 3D Digital PCR System (Thermo Fisher), QIAcuity Digital PCR System (QIAGEN), Droplet Digital PCR System (Bio-Rad), Naica Crystal Digital PCR System (Stilla Technologies), RainDrop digital PCR system (RainDance Technologies, Bio-rad), BioMark digital PCR system (Fluidigm) and so on. Crude samples, without initial step of nucleic acid extraction, could also be used for single-cell detection. As RADICA only requires one temperature for the reaction, it can be integrated with a portable heater and smartphone-based fluorescence detection for point-of-care quantification. Based on the superior performance in sensitivity, speed, inhibitor resistance, and quantitative detection and the great potential for improvement and applicability, we anticipate that RADICA will be a promising quantitative molecular tool applicable to the clinical setting, as well as to research, biomanufacturing, and environmental and food industries.

### REFERENCES

1. ADDIN EN. REFLIST Alteri, C., Cento, V., Antonello, M., Colagrossi, L., Merli, M., Ughi, N., Renica, S., Matarazzo, E., Di Ruscio, F., Tartaglione, L., Colombo, J., Grimaldi, C., Carta, S., Nava, A., Costabile, V., Baiguera, C., Campisi, D., Fanti, D., Vismara, C., Fumagalli, R., Scaglione, F., Epis, O. M., Puoti, M., Perno, C. F., 2020. Detection and quantification of SARS-CoV-2 by droplet digital PCR in real-time PCR negative nasopharyngeal swabs from suspected COVID-19 patients. PloS one 15(9), e0236311.
2. Barone, P. W., Wiebe, M. E., Leung, J. C., Hussein, I. T. M., Keumurian, F. J., Bouressa, J., Brussel, A., Chen, D., Chong, M., Dehghani, H., Gerentes, L., Gilbert, J., Gold, D., Kiss, R., Kreil, T. R., Labatut, R., Li, Y., Mullberg, J., Mallet, L., Menzel, C., Moody, M., Monpoeho, S., Murphy, M., Playsic, M., Roth, N.J., Roush, D., Ruffing, M., Schicho, R., Snyder, R., Stark, D., Zhang, C., Wolfrum, J., Sinskey, A. J., Springs, S. L., 2020. Viral contamination in biologic manufacture and implications for emerging therapies. Nature biotechnology 38(5), 563-572.
3. Broughton, J. P., Deng, X., Yu, G., Fasching, C. L., Servellita, V., Singh, J., Miao, X., Streithorst, J. A., Granados, A., Sotomayor-Gonzalez, A., Zorn, K., Gopez, A., Hsu, E., Gu, W., Miller, S., Pan, C.-Y., Guevara, H., Wadford, D. A., Chen, J. S., Chiu, C. Y., 2020. CRISPR-Cas12-based detection of SARS-CoV-2. Nature biotechnology.
4. Chen, J. S., Ma, E., Harrington, L. B., Da Costa, M., Tian, X., Palefsky, J. M., Doudna, J. A., 2018. CRISPR-Cas12a target binding unleashes indiscriminate single-stranded DNase activity. Science 360(6387), 436-439.
5. Ding, X., Yin, K., Li, Z., Lalla, R. V., Ballesteros, E., Sfeir, M. M., Liu, C., 2020. Ultrasensitive and visual detection of SARS-CoV-2 using all-in-one dual CRISPR-Cas12a assay. Nature communications 11(1), 4711.
6. Dingle, T. C., Sedlak, R. H., Cook, L., Jerome, K. R., 2013. Tolerance of Droplet-Digital PCR vs Real-Time Quantitative PCR to Inhibitory Substances. Clinical chemistry 59(11), 1670-1672.
7. Dong, L., Meng, Y., Sui, Z., Wang, J., Wu, L., Fu, B., 2015. Comparison of four digital PCR platforms for accurate quantification of DNA copy number of a certified plasmid DNA reference material. Scientific reports 5(1), 13174.
8. Gill, P., Hadian Amree, A., 2020. AS-LAMP: A New and Alternative Method for Genotyping. Avicenna J Med Biotechnol 12(1), 2-8.
9. Gootenberg, J. S., Abudayyeh, O. O., Kellner, M. J., Joung, J., Collins, J. J., Zhang, F., 2018. Multiplexed and portable nucleic acid detection platform with Cas13, Cas12a, and Csm6. Science 360(6387), 439-444.
10. Gootenberg, J. S., Abudayyeh, O. O., Lee, J. W., Essletzbichler, P., Dy, A. J., Joung, J., Verdine, V., Donghia, N., Daringer, N. M., Freije, C. A., Myhrvold, C., Bhattacharyya, R. P., Livny, J., Regev, A., Koonin, E. V., Hung, D. T., Sabeti, P. C., Collins, J. J., Zhang, F., 2017. Nucleic acid detection with CRISPR-Cas13a/C2c2. Science 356(6336), 438-442.
11. Joung, J., Ladha, A., Saito, M., Kim, N.-G., Woolley, A. E., Segel, M., Barretto, R. P. J., Ranu, A., Macrae, R. K., Faure, G., loannidi, E. I., Krajeski, R. N., Bruneau, R., Huang, M.-L. W., Yu, X. G., Li, J. Z., Walker, B. D., Hung, D. T., Greninger, A. L., Jerome, K. R., Gootenberg, J. S., Abudayyeh, O. O., Zhang, F., 2020. Detection of SARS-CoV-2 with SHERLOCK One-Pot Testing. New England Journal of Medicine.
12. Kellner, M. J., Koob, J. G., Gootenberg, J. S., Abudayyeh, O. O., Zhang, F., 2019. SHERLOCK: nucleic acid detection with CRISPR nucleases. Nature protocols 14(10), 2986-3012.
13. Li, J., Macdonald, J., von Stetten, F., 2018. Review: a comprehensive summary of a decade development of the recombinase polymerase amplification. The Analyst 144(1), 31-67.
14. Li, S. Y., Cheng, Q. X., Liu, J. K., Nie, X. Q., Zhao, G. P., Wang, J., 2018. CRISPR-Cas12a has both cis- and trans-cleavage activities on single-stranded DNA. Cell research 28(4), 491-493.
15. Li, S. Y., Cheng, Q. X., Wang, J. M., Li, X. Y., Zhang, Z. L., Gao, S., Cao, R. B., Zhao, G. P., Wang, J., 2018. CRISPR-Cas12a-assisted nucleic acid detection. Cell discovery 4, 20.
16. Liu, X., Feng, J., Zhang, Q., Guo, D., Zhang, L., Suo, T., Hu, W., Guo, M., Wang, X., Huang, Z., Xiong, Y., Chen, G., Chen, Y., Lan, K., 2020. Analytical comparisons of SARS-COV-2 detection by qRT-PCR and ddPCR with multiple primer/probe sets. Emerging microbes & infections 9(1), 1175-1179.
17. Liu, Y., Ning, Z., Chen, Y., Guo, M., Liu, Y., Gali, N. K., Sun, L., Duan, Y., Cai, J., Westerdahl, D., Liu, X., Xu, K., Ho, K.-f., Kan, H., Fu, Q., Lan, K., 2020. Aerodynamic analysis of SARS-CoV-2 in two Wuhan hospitals. Nature 582(7813), 557-560.
18. Lizardi, P. M., Huang, X., Zhu, Z., Bray-Ward, P., Thomas, D.C., Ward, D. C., 1998. Mutation detection and single-molecule counting using isothermal rolling-circle amplification. Nature Genetics 19(3), 225-232.
19. Lucia, C., Federico, P.-B., Alejandra, G. C., 2020. An ultrasensitive, rapid, and portable coronavirus SARS-CoV-2 sequence detection method based on CRISPR-Cas12. bioRxiv, 2020.2002.2029.971127.
20. Notomi, T., Okayama, H., Masubuchi, H., Yonekawa, T., Watanabe, K., Amino, N., Hase, T., 2000. Loop-mediated isothermal amplification of DNA. Nucleic acids research 28(12), E63-E63.
21. Piepenburg, O., Williams, C. H., Stemple, D. L., Armes, N. A., 2006. DNA Detection Using Recombination Proteins. PLOS Biology 4(7), e204.
22. Rohrman, B., Richards-Kortum, R., 2015. Inhibition of recombinase polymerase amplification by background DNA: a lateral flow-based method for enriching target DNA. Analytical chemistry 87(3), 1963-1967.
23. Salipante, S. J., Jerome, K. R., 2020. Digital PCR—An Emerging Technology with Broad Applications in Microbiology. Clinical chemistry 66(1), 117-123.
24. Sedlak, R. H., Jerome, K. R., 2013. Viral diagnostics in the era of digital polymerase chain reaction. Diagnostic microbiology and infectious disease 75(1), 1-4.
25. Shu, Y., McCauley, J., 2017. GISAID: Global initiative on sharing all influenza data—from vision to reality. Eurosurveillance 22(13), 30494.
26. Suo, T., Liu, X., Feng, J., Guo, M., Hu, W., Guo, D., Ullah, H., Yang, Y., Zhang, Q., Wang, X., Sajid, M., Huang, Z., Deng, L., Chen, T., Liu, F., Xu, K., Liu, Y., Zhang, Q., Liu, Y., Xiong, Y., Chen, G., Lan, K., Chen, Y., 2020. ddPCR: a more sensitive and accurate tool for SARS-CoV-2 detection in low viral load specimens. Emerging microbes & infections 9(1), 1259-1268.
27. Tay, J. K., Siow, C. H., Goh, H. L., Lim, C. M., Hsu, P. P., Chan, S. H., Loh, K. S., 2020. A comparison of EBV serology and serum cell-free DNA as screening tools for nasopharyngeal cancer: Results of the Singapore NPC screening cohort. International Journal of Cancer 146(10), 2923-2931.
28. Tomita, N., Mori, Y., Kanda, H., Notomi, T., 2008. Loop-mediated isothermal amplification (LAMP) of gene sequences and simple visual detection of products. Nature protocols 3(5), 877-882.
29. Vo, J. H., Nei, W. L., Hu, M., Phyo, W. M., Wang, F., Fong, K. W., Tan, T., Soong, Y. L., Cheah, S. L., Sommat, K., Low, H., Ling, B., Ng, J., Tan, W. L., Chan, K. S., Oon, L., Ying, J. Y., Tan, M.-H., 2016. Comparison of Circulating Tumour Cells and Circulating Cell-Free Epstein-Barr Virus DNA in Patients with Nasopharyngeal Carcinoma Undergoing Radiotherapy. Scientific reports 6(1), 13-13.
30. Walker, G. T., Fraiser, M. S., Schram, J. L., Little, M. C., Nadeau, J. G., Malinowski, D. P., 1992. Strand displacement amplification—an isothermal, in vitro DNA amplification technique. Nucleic acids research 20(7), 1691-1696.
31. Whale, A. S., Cowen, S., Foy, C. A., Huggett, J. F., 2013. Methods for Applying Accurate Digital PCR Analysis on Low Copy DNA Samples. PloS one 8(3), e58177.
32. Whale, A. S., Huggett, J. F., Cowen, S., Speirs, V., Shaw, J., Ellison, S., Foy, C. A., Scott, D. J., 2012. Comparison of microfluidic digital PCR and conventional quantitative PCR for measuring copy number variation. Nucleic acids research 40(11), e82-e82.
33. Yao, X.-H., He, Z.-C., Li, T.-Y., Zhang, H.-R., Wang, Y., Mou, H., Guo, Q., Yu, S.-C., Ding, Y., Liu, X., Ping, Y.-F., Bian, X.-W., 2020. Pathological evidence for residual SARS-CoV-2 in pulmonary tissues of a ready-for-discharge patient. Cell research 30(6), 541-543.
34. Yu, F., Yan, L., Wang, N., Yang, S., Wang, L., Tang, Y., Gao, G., Wang, S., Ma, C., Xie, R., Wang, F., Tan, C., Zhu, L., Guo, Y., Zhang, F., 2020. Quantitative Detection and Viral Load Analysis of SARS-CoV-2 in Infected Patients. Clinical Infectious Diseases 71(15), 793-798.
35. Zhang, Y., Ren, G., Buss, J., Barry, A. J., Patton, G. C., Tanner, N. A., 2020. Enhancing colorimetric loop-mediated isothermal amplification speed and sensitivity with guanidine chloride. Biotechniques 69(3), 178-185.

1. A method for detecting and quantitating target nucleic acids in a sample comprising:
a) forming a mixture comprising sample nucleic acids;
isothermal amplification reaction reagents for amplifying one or more target nucleic acid sequences;
a Cas12a, Cas12b, Cas13b or Cas14 effector, or a derivative thereof;
at least one guide polynucleotide comprising a DNA-targeting sequence, and designed to form a complex with the Cas effector; and
a nucleic acids-based masking construct comprising a non-target sequence,
b) partitioning the mixture into a plurality of compartments;
c) incubating the partitioned mixture at a temperature for isothermal amplification and Cas effector cleavage of an amplified DNA strand,
wherein the Cas effector exhibits collateral nuclease activity and cleaves the non-target sequence of the nucleic acids-based masking construct once activated by the target sequences; and
d) detecting a signal from cleavage of the non-target sequence, thereby detecting the one or more target sequences in the sample, and
e) determining the copy number of the target nucleic acid based on a Poisson distribution of the proportion of positive-to-negative compartments.

2. The method of claim 1, wherein:
i) the Cas effector is Cas12a or Cas12b; and/or
ii) the method is used to detect and/or quantify a pathogen, gene expression, gene copy number variation or adventitious agents in a sample; and/or
iii) the at least one guide polynucleotide is crRNA.

3. (canceled)

4. (canceled)

5. The method of claim 1, wherein the amplification is selected from the group comprising nucleic-acid sequence-based amplification, recombinase polymerase amplification, loop-mediated isothermal amplification, strand-displacement amplification, exonuclease Ill-assisted signal amplification, hybridization chain reaction, helicase-dependent amplification, isothermal circular strand displacement polymerization, multiple displacement amplification, primase-based whole genome amplification, rolling circle amplification and whole genome amplification.

6. The method of claim 1, wherein the isothermal amplification is:
a) selected from the group comprising recombinase polymerase amplification, strand-displacement amplification, rolling circle amplification and multiple displacement amplification; the Cas effector is Cas12a, or
b) selected from the group comprising loop-mediated isothermal amplification, helicase-dependent amplification, strand-displacement amplification and rolling circle amplification; the Cas effector is Cas12b.

7. The method of claim 1, wherein the masking construct suppresses generation of a detectable positive signal until cleaved, or masks a detectable positive signal until the masking construct is cleaved.

8. The method of claim 1, wherein;
i) the masking construct comprises a quenched fluorescent nucleic acids probe, such as a ssDNA probe, dsDNA or RNA probe; and/or
ii) the target is DNA or RNA, such as virus DNA or RNA; and\or
iii) the virus is SARS-CoV-2 virus, human adenovirus (HAdV), herpes simplex virus (HSV), or Epstein-Barr virus (EBV); and/or
iv) the partitioning is microfluidics-based, droplets-based or membrane-based; and\or
v) the mixture is partitioned into at least 1,000 compartments.

9.-12. (canceled)

13. The method of claim 1, wherein the guide has a sequence comprising a mismatch to the one or more target sequences.

14. The method of claim 1, wherein the isothermal amplification is a warm-start LAMP or RT-LAMP reaction and/or a multiplex reaction.

15. The method of claim 1, wherein:
the target nucleic acids are SARS-CoV-2, HAdV, herpes simplex virus (HSV), or Epstein-Barr virus nucleic acids;
the isothermal amplification is:
a) recombinase polymerase amplification; the Cas effector is Cas12a, or
b) LAMP or RT-LAMP; the Cas effector is Cas12b;
the at least one guide polynucleotide is crRNA;
the mixture is partitioned into at least 1,000 compartments and is chip-based; and
the masking construct comprises a quenched fluorescent ssDNA probe.

16. A method for detecting the presence and/or severity of a disease in a subject, comprising the steps of:
a) forming a mixture comprising a sample comprising nucleic acids from the subject;
isothermal amplification reaction reagents for amplifying one or more target disease nucleic acid sequences;
a Cas12a, Cas12b, Cas13b or Cas14 effector, or a variant thereof;
at least one guide polynucleotide comprising a DNA-targeting sequence, and designed to form a complex with the Cas effector; and
a nucleic acids-based masking construct comprising a non-target sequence,
b) partitioning the mixture into compartments;
c) incubating the partitioned mixture at a temperature for isothermal amplification and Cas effector cleavage of an amplified DNA strand,
wherein the Cas effector exhibits collateral nuclease activity and cleaves the non-target sequence of the nucleic acids-based masking construct once activated by the target sequences;
d) detecting a signal from cleavage of the non-target sequence, thereby detecting the one or more target sequences in the sample;
e) determining the copy number of the target nucleic acid based on a Poisson distribution of the proportion of positive-to-negative compartments and comparing the number to a control value;
wherein positive compartments indicate the presence of disease in said subject, and wherein the copy number of the target nucleic acid indicates the severity of the disease in said subject.

17. The method of claim 16, wherein;
i) the disease is a pathogen infection, such as a virus infection; and\or
ii) the Cas effector is Cas12a or Cas12b; and\or
iii) the method is used to detect and/or quantify a pathogen, gene expression or gene copy number variation; and\or;
iv) the at least one guide polynucleotide is crRNA.

18.-20. (canceled)

21. The method of claim 16, wherein the amplification is selected from the group comprising nucleic-acid sequence-based amplification, recombinase polymerase amplification, loop-mediated isothermal amplification, strand-displacement amplification, exonuclease Ill-assisted signal amplification, hybridization chain reaction, helicase-dependent amplification, isothermal circular strand displacement polymerization, multiple displacement amplification, primase-based whole genome amplification, rolling circle amplification and whole genome amplification.

22. The method of claim 16, wherein the isothermal amplification is:
a) selected from the group comprising recombinase polymerase amplification, strand-displacement amplification, rolling circle amplification and multiple displacement amplification; the Cas effector is Cas12a, or
b) selected from the group comprising loop-mediated isothermal amplification, helicase-dependent amplification, strand-displacement amplification and rolling circle amplification; the Cas effector is Cas12b.

23. The method of claim 16, wherein the masking construct suppresses generation of a detectable positive signal until cleaved, or masks a detectable positive signal until the masking construct is cleaved.

24. The method of claim 23, wherein the masking construct comprises a quenched fluorescent nucleic acids probe.

25. The method of claim 16, wherein the target is DNA or RNA, such as virus DNA or RNA.

26. The method of claim 25, wherein the virus is SARS-CoV-2, human adenovirus (HAdV), herpes simplex virus (HSV), or Epstein-Barr virus.

27. The method of claim 16, wherein the partitioning is microfluidics-based, droplets-based or membrane-based.

28. The method of claim 16, wherein the mixture is partitioned into at least 1,000 compartments.

29. The method of claim 16, wherein the isothermal amplification is a warm-start RT-LAMP reaction and/or a multiplex reaction.

30. The method of claim 16, further comprising administering a treatment that is efficacious for the severity of the disease in said subject.

31. A kit to quantitate target nucleic acids in a sample comprising:
a) isothermal amplification reaction reagents for amplifying one or more target nucleic acid sequences;
b) a Cas12a, Cas12b, Cas13b or Cas14 effector, or a variant thereof;
c) at least one guide polynucleotide comprising a DNA-targeting sequence, and designed to form a complex with the Cas effector;
d) a nucleic acids-based masking construct comprising a non-target sequence, and
e) a partitioning device or substrate.

32. The kit of claim 31, wherein the target nucleic acids are SARS-CoV-2, human adenovirus (HAdV), herpes simplex virus (HSV), or Epstein-Barr virus nucleic acids; the isothermal amplification reaction reagents are:
a) recombinase polymerase amplification reaction reagents; the Cas effector is Cas12a; or
b) warm-start RT-LAMP amplification reaction reagents; the Cas effector is Cas12b;
the at least one guide polynucleotide is crRNA; the partitioning device or substrate comprises at least 1,000 compartments and is chip-based; and the nucleic acids-based masking construct comprises at least one quenched fluorescent ssDNA probe.


SEQUENCE LISTING (339 sequences)

SEQ ID NO: 1 - DNA, 49 bp, Artificial Sequence
  N-RNA forward primer
gaaattaatacgactcactatagggatgtctgataatggaccccaaaat

SEQ ID NO: 2 - DNA, 29 bp, Artificial Sequence
  R-RNA reverse primer
gaaatttaggcctgagttgagtcagcact

SEQ ID NO: 3 - DNA, 22 bp, Artificial Sequence
  dPCR Primer for N gene
ggggaacttctcctgctagaat

SEQ ID NO: 4 - DNA, 22 bp, Artificial Sequence
  dPCR Primer for N gene
cagacattttgctctcaagctg

SEQ ID NO: 5 - DNA, 20 bp, Artificial Sequence
  dPCR Probe for N gene
ttgctgctgcttgacagatt

SEQ ID NO: 6 - DNA, 26 bp, Artificial Sequence
  CRISPR Primer for N gene
aactccaggcagcagtaggggaactt

SEQ ID NO: 7 - DNA, 26 bp, Artificial Sequence
  CRISPR Primer for N gene
cctttaccagacattttgctctcaag

SEQ ID NO: 8 - DNA, 63 bp, Artificial Sequence
  crRNA template for N gene, pair with T7-3G IVT primer
aatctgtcaagcagcagatctacacttagtagaaattaccctatagtgagtcgtattaatttc

SEQ ID NO: 9 - DNA, 33 bp, Artificial Sequence
  RADICA Primer for N gene, N1 region
aggcagcagtaggggaacttctcctgctagaat

SEQ ID NO: 10 - DNA, 33 bp, Artificial Sequence
  RADICA Primer for N gene, N1 region
ttggcctttaccagacattttgctctcaagctg

SEQ ID NO: 11 - DNA, 66 bp, Artificial Sequence
  crRNA template for N gene, pair with T7-3G IVT primer
ggctggcaatggcggtgatgatctacacttagtagaaattaccctatagtgagtcgtattaatttc

SEQ ID NO: 12 - DNA, 25 bp, Artificial Sequence
  Universal primer used for crRNA synthesis
gaaattaatacgactcactataggg

SEQ ID NO: 13 - DNA, 33 bp, Artificial Sequence
  CRISPR Primer for N gene
agcagatttcttagtgacagtttggccttgttg

SEQ ID NO: 14 - DNA, 45 bp, Artificial Sequence
  T7-Cas12scaffold forward primer
gaaattaatacgactcactataggtaatttctactaagtgtagat

SEQ ID NO: 15 - DNA, 32 bp, Artificial Sequence
  RADICA Primer for N gene, N0 region
caacttcctcaaggaacaacattgccaaaagg

SEQ ID NO: 16 - DNA, 32 bp, Artificial Sequence
  RADICA Primer for N gene, N0 region
tggagttgaatttcttgaactgttgcgactac

SEQ ID NO: 17 - DNA, 32 bp, Artificial Sequence; identical to SEQ ID NO: 16
  RADICA Primer for N gene, N0 region

SEQ ID NO: 18 - DNA, 41 bp, Artificial Sequence
  crRNA template for N gene, , N0 region, pair with T7-Cas12scaffold-F
cttctacgcagaagggagcaatctacacttagtagaaatta

SEQ ID NO: 19 - DNA, 20 bp, Artificial Sequence
  qPCR and dPCR  primer for EBV clinical sample
tcatcatcatccgggtctcc

SEQ ID NO: 20 - DNA, 18 bp, Artificial Sequence
  qPCR and dPCR  primer for EBV clinical sample
gctcaccatctgggccac

SEQ ID NO: 21 - DNA, 34 bp, Artificial Sequence
  qPCR and dPCR  probe for EBV clinical sample
cctccaggtagaaggccatttttccaccctgtag

SEQ ID NO: 22 - DNA, 19 bp, Artificial Sequence
  qPCR and dPCR  primer for EBV clinical sample
cccaacactccaccacacc

SEQ ID NO: 23 - DNA, 20 bp, Artificial Sequence
  qPCR and dPCR  primer for EBV clinical sample
tcttaggagctgtccgaggg

SEQ ID NO: 24 - DNA, 27 bp, Artificial Sequence
  qPCR and dPCR  probe for EBV clinical sample
cacacactacacacacccacccgtctc

SEQ ID NO: 25 - DNA, 32 bp, Artificial Sequence
  RADICA forward primer for EBV EBNA-1
gccggtgtgttcgtatatggaggtagtaagac

SEQ ID NO: 26 - DNA, 31 bp, Artificial Sequence
  RADICA reverse primer for EBV EBNA-1
attccaaaggggagacgactcaatggtgtaa

SEQ ID NO: 27 - DNA, 43 bp, Artificial Sequence
  crRNA template for EBV EBNA-1, pair with T7-Cas12scaffold-F
acgacattgtggaayagcaaggatctacacttagtagaaatta

SEQ ID NO: 28 - DNA, 33 bp, Artificial Sequence
  RADICA forward primer for EBV BamHI-W
ctgcccctggtataaagtggtcctgcagctatt

SEQ ID NO: 29 - DNA, 32 bp, Artificial Sequence
  RADICA reverse primer for EBV BamHI-W
ggctagggagaggtagaagaccccctcttaca

SEQ ID NO: 30 - skipped

SEQ ID NO: 31 - DNA, 33 bp, Artificial Sequence
  RPA-RNAseP primer
aaattacatctggtctcttccttcactgcttca

SEQ ID NO: 32 - DNA, 33 bp, Artificial Sequence
  RPA-RNAseP primer
taaattatttccaaagttggttcagtccgatgc

SEQ ID NO: 33 - DNA, 34 bp, Artificial Sequence
  RPA-RNAseP primer
gccagatgtttgaatattttaagagcttctttcg

SEQ ID NO: 34 - DNA, 33 bp, Artificial Sequence
  RPA-RNAseP primer
tgaagcagtgaaggaagagaccagatgtaattt

SEQ ID NO: 35 - DNA, 34 bp, Artificial Sequence
  RPA-RNAseP primer
tgttttaagcttctttcatgtattcaaatcagca

SEQ ID NO: 36 - DNA, 33 bp, Artificial Sequence
  RPA-RNAseP primer
catgtgtatcctctctccttccacaaattctat

SEQ ID NO: 37 - DNA, 33 bp, Artificial Sequence
  RPA-RNAseP primer
tgcaatattaatgtaagggctctaaaacaatgg

SEQ ID NO: 38 - DNA, 33 bp, Artificial Sequence
  RPA-RNAseP primer
aaaaattgtattttctccaacccgcagaacagt

SEQ ID NO: 39 - DNA, 41 bp, Artificial Sequence
  RNAseP Cas12a crRNA template
tgcctacgtaaggtctttgaatctacacttagtagaaatta

SEQ ID NO: 40 - DNA, 41 bp, Artificial Sequence
  RNAseP Cas12a crRNA template
tttgctatttttaatacagcatctacacttagtagaaatta

SEQ ID NO: 41 - DNA, 41 bp, Artificial Sequence
  RNAseP Cas12a crRNA template
aaagtttcttgttcatactcatctacacttagtagaaatta

SEQ ID NO: 42 - DNA, 41 bp, Artificial Sequence
  RNAseP Cas12a crRNA template
tacatgtgtatcctctctccatctacacttagtagaaatta

SEQ ID NO: 43 - DNA, 41 bp, Artificial Sequence
  RNAseP Cas12a crRNA template
tttttttctaagaaattgctatctacacttagtagaaatta

SEQ ID NO: 44 - DNA, 41 bp, Artificial Sequence
  RNAseP Cas12a crRNA template
agcaatttcttagaaaaaaaatctacacttagtagaaatta

SEQ ID NO: 45 - DNA, 41 bp, Artificial Sequence
  RNAseP Cas12a crRNA template
agtagagccagaggtataacatctacacttagtagaaatta

SEQ ID NO: 46 - DNA, 41 bp, Artificial Sequence
  RNAseP Cas12a crRNA template
tgtctttctcttgcttaaaaatctacacttagtagaaatta

SEQ ID NO: 47 - DNA, 12 bp, Artificial Sequence
  FQ reporter
tttttttttttt

SEQ ID NO: 48 - DNA, 16 bp, Artificial Sequence
  FQ reporter
tttttttttttttttt

SEQ ID NO: 49 - DNA, 20 bp, Artificial Sequence
  FQ reporter
tttttttttttttttttttt

SEQ ID NO: 50 - DNA, 111 bp, Artificial Sequence
  SARS-CoV-2 crRNA for Cas12b
gucuagaggacagaauuuuucaacgggugugccaauggccacuuuccagguggcaaagcccguugagcuucucaaaucugagaaguggcaccgaagaacgcugaagcgcug

SEQ ID NO: 51 - DNA, 18 bp, Artificial Sequence
  SARS-CoV-2 LAMP primer
gctgctgaggcttctaag

SEQ ID NO: 52 - DNA, 21 bp, Artificial Sequence
  SARS-CoV-2 LAMP primer
gcgtcaatatgcttattcagc

SEQ ID NO: 53 - DNA, 40 bp, Artificial Sequence
  SARS-CoV-2 LAMP primer
gcggccaatgtttgtaatcagtagacgtggtccagaacaa

SEQ ID NO: 54 - DNA, 39 bp, Artificial Sequence
  SARS-CoV-2 LAMP primer
tcagcgttcttcggaatgtcgctgtgtaggtcaaccacg

SEQ ID NO: 55 - DNA, 22 bp, Artificial Sequence
  SARS-CoV-2 LAMP primer
ccttgtctgattagttcctggt

SEQ ID NO: 56 - DNA, 18 bp, Artificial Sequence
  SARS-CoV-2 LAMP primer
tggcatggaagtcacacc

SEQ ID NO: 57 - DNA, 20 bp, Artificial Sequence
  HSV LAMP primer
rccgacgtgtactaytacga

SEQ ID NO: 58 - DNA, 17 bp, Artificial Sequence
  HSV LAMP primer
ggytgggcyrgcgygtt

SEQ ID NO: 59 - DNA, 37 bp, Artificial Sequence
  HSV LAMP primer
sgggcaraagttgtcgcacataccgcgtctwcgtscg

SEQ ID NO: 60 - DNA, 39 bp, Artificial Sequence
  HSV LAMP primer
tcargaagtacgagggkggsgtcggtaccagccgaaggt

SEQ ID NO: 61 - DNA, 21 bp, Artificial Sequence
  HSV LAMP primer
gtasgycagckcgcgcccgct

SEQ ID NO: 62 - DNA, 20 bp, Artificial Sequence
  HSV LAMP primer
ccggttyatcctggacaacc

SEQ ID NO: 63 - DNA, 19 bp, Artificial Sequence
  HSV LAMP primer
ccmccgtcaccgtcttyca

SEQ ID NO: 64 - DNA, 19 bp, Artificial Sequence
  HSV LAMP primer
tcvacctcckccttgttca

SEQ ID NO: 65 - DNA, 38 bp, Artificial Sequence
  HSV LAMP primer
aaycgckcgtggarctgggcgtgtaygacatcctggag

SEQ ID NO: 66 - DNA, 39 bp, Artificial Sequence
  HSV LAMP primer
ggaccgtcatcacgctyctggcgtgccgtaracgtgaac

SEQ ID NO: 67 - DNA, 17 bp, Artificial Sequence
  HSV LAMP primer
cgcatgcygtacgcgtg

SEQ ID NO: 68 - DNA, 20 bp, Artificial Sequence
  HSV LAMP primer
ggyctgacyccsgaaggcca

SEQ ID NO: 69 - DNA, 18 bp, Artificial Sequence
  HSV LAMP primer
cgcgtctwcgtscgaagc

SEQ ID NO: 70 - DNA, 17 bp, Artificial Sequence
  HSV LAMP primer
ggytgggcyrgcgygtt

SEQ ID NO: 71 - DNA, 38 bp, Artificial Sequence
  HSV LAMP primer
gacsccmccctcgtacttcytgcgcgygctgkcstacc

SEQ ID NO: 72 - DNA, 37 bp, Artificial Sequence
  HSV LAMP primer
cgccaccacccggttyatgccsggyttgagrcggtac

SEQ ID NO: 73 - DNA, 19 bp, Artificial Sequence
  HSV LAMP primer
gggcagaagttgtcgcaca

SEQ ID NO: 74 - DNA, 20 bp, Artificial Sequence
  HSV LAMP primer
gggttygtcaccttcggctg

SEQ ID NO: 75 - DNA, 16 bp, Artificial Sequence
  HSV LAMP primer
rcacgcgtacrgcatg

SEQ ID NO: 76 - DNA, 17 bp, Artificial Sequence
  HSV LAMP primer
cgctcgcagagatckcg

SEQ ID NO: 77 - DNA, 37 bp, Artificial Sequence
  HSV LAMP primer
agrcccagragcgtgatgacacgmgcgrtttatggac

SEQ ID NO: 78 - DNA, 37 bp, Artificial Sequence
  HSV LAMP primer
sgccgttcacgtytacggcagcvcggcaytgyaggtg

SEQ ID NO: 79 - DNA, 17 bp, Artificial Sequence
  HSV LAMP primer
gtcccsgysggygtgat

SEQ ID NO: 80 - DNA, 25 bp, Artificial Sequence
  HSV LAMP primer
gcggcagtacttttacatgaacaag

SEQ ID NO: 81 - DNA, 20 bp, Artificial Sequence
  HSV LAMP primer
cgtcttycacgtgtaygaca

SEQ ID NO: 82 - DNA, 16 bp, Artificial Sequence
  HSV LAMP primer
ggcvcggcaytgyagg

SEQ ID NO: 83 - DNA, 39 bp, Artificial Sequence
  HSV LAMP primer
ccataaaycgckcgtggarctctggagmacgtggarcac

SEQ ID NO: 84 - DNA, 39 bp, Artificial Sequence
  HSV LAMP primer
ctgacyccsgaaggccayccvacctcckccttgttcatg

SEQ ID NO: 85 - DNA, 17 bp, Artificial Sequence
  HSV LAMP primer
gcgcgcatgcygtacgc

SEQ ID NO: 86 - DNA, 19 bp, Artificial Sequence
  HSV LAMP primer
cggcacgcggcagtacttt

SEQ ID NO: 87 - DNA, 20 bp, Artificial Sequence
  HSV LAMP primer
agatgytgttggccttcatg

SEQ ID NO: 88 - DNA, 18 bp, Artificial Sequence
  HSV LAMP primer
aagtggctctggcckatg

SEQ ID NO: 89 - DNA, 39 bp, Artificial Sequence
  HSV LAMP primer
cagcttggycagsaygaagggaarcagtacggccccgag

SEQ ID NO: 90 - DNA, 39 bp, Artificial Sequence
  HSV LAMP primer
tgacggasatytacaaggtccctcccacacgcgraacac

SEQ ID NO: 91 - DNA, 24 bp, Artificial Sequence
  HSV LAMP primer
tgatgatgttgtacccggtcacga

SEQ ID NO: 92 - DNA, 20 bp, Artificial Sequence
  HSV LAMP primer
tacggscgcatgaacggccg

SEQ ID NO: 93 - DNA, 17 bp, Artificial Sequence
  HSV LAMP primer
ggygtgttycgcgtgtg

SEQ ID NO: 94 - DNA, 16 bp, Artificial Sequence
  HSV LAMP primer
atcaccccgcgytgcg

SEQ ID NO: 95 - DNA, 41 bp, Artificial Sequence
  HSV LAMP primer
tgttcaccatsccgttcaccttggacatmggccagagccac

SEQ ID NO: 96 - DNA, 39 bp, Artificial Sequence
  HSV LAMP primer
tcsagctacaagctsaacgccgcggggatgtcgcgrtag

SEQ ID NO: 97 - DNA, 19 bp, Artificial Sequence
  HSV LAMP primer
tcttgctgcgcttctgraa

SEQ ID NO: 98 - DNA, 23 bp, Artificial Sequence
  HSV LAMP primer
gtcytgaaggacaagaagaagga

SEQ ID NO: 99 - DNA, 19 bp, Artificial Sequence
  HSV LAMP primer
gccgtcytgaaggacaaga

SEQ ID NO: 100 - DNA, 16 bp, Artificial Sequence
  HSV LAMP primer
ratrcccgccargcgc

SEQ ID NO: 101 - DNA, 39 bp, Artificial Sequence
  HSV LAMP primer
tactcgccgatcaccccgcagaaggayctgagctaycgc

SEQ ID NO: 102 - DNA, 38 bp, Artificial Sequence
  HSV LAMP primer
caggaytcsctgctggtsggggcsgaragctccagrtg

SEQ ID NO: 103 - DNA, 19 bp, Artificial Sequence
  HSV LAMP primer
gcgtagtaggcggggatgt

SEQ ID NO: 104 - DNA, 18 bp, Artificial Sequence
  HSV LAMP primer
cagctgttttttaagttt

SEQ ID NO: 105 - DNA, 20 bp, Artificial Sequence
  HSV LAMP primer
gccttcatgaccytygtsaa

SEQ ID NO: 106 - DNA, 18 bp, Artificial Sequence
  HSV LAMP primer
gctgcgcttctgraagtg

SEQ ID NO: 107 - DNA, 37 bp, Artificial Sequence
  HSV LAMP primer
cagcttggycagsamgaagggggccccgagttcgtga

SEQ ID NO: 108 - DNA, 40 bp, Artificial Sequence
  HSV LAMP primer
acggasatytacaaggtcccsctggcckatgtcccacacg

SEQ ID NO: 109 - DNA, 25 bp, Artificial Sequence
  HSV LAMP primer
ccagtcgaagttgatgatgttgtac

SEQ ID NO: 110 - DNA, 25 bp, Artificial Sequence
  HSV LAMP primer
ccagtcgaagttgatgatgttgtac

SEQ ID NO: 111 - DNA, 17 bp, Artificial Sequence
  HSV LAMP primer
ycagaagcgcagcaaga

SEQ ID NO: 112 - DNA, 18 bp, Artificial Sequence
  HSV LAMP primer
csaccagcagsgartcct

SEQ ID NO: 113 - DNA, 41 bp, Artificial Sequence
  HSV LAMP primer
ggcgttsagcttgtagctsgagtgaacggsatggtgaacat

SEQ ID NO: 114 - DNA, 39 bp, Artificial Sequence
  HSV LAMP primer
yctgagctaycgcgacatccctactcgccgatcaccccg

SEQ ID NO: 115 - DNA, 23 bp, Artificial Sequence
  HSV LAMP primer
cggtkatratsccgtacatgtcg

SEQ ID NO: 116 - DNA, 21 bp, Artificial Sequence
  HSV LAMP primer
ctacgcckccgggcccgcgca

SEQ ID NO: 117 - DNA, 17 bp, Artificial Sequence
  HSV LAMP primer
scgacggctgttcttcg

SEQ ID NO: 118 - DNA, 17 bp, Artificial Sequence
  HSV LAMP primer
cacyccsgtgaacccgt

SEQ ID NO: 119 - DNA, 37 bp, Artificial Sequence
  HSV LAMP primer
tttcgcatggcsagccagtcsaaggcycacgtrcgmg

SEQ ID NO: 120 - DNA, 40 bp, Artificial Sequence
  HSV LAMP primer
cggatyccccagagcascccaccgagttrcacacsacctt

SEQ ID NO: 121 - DNA, 16 bp, Artificial Sequence
  HSV LAMP primer
aggatgctsagsaggc

SEQ ID NO: 122 - DNA, 19 bp, Artificial Sequence
  HSV LAMP primer
aggccgtsctcctsgacaa

SEQ ID NO: 123 - DNA, 19 bp, Artificial Sequence
  HSV LAMP primer
tcttcgtsaaggcycacgt

SEQ ID NO: 124 - DNA, 19 bp, Artificial Sequence
  HSV LAMP primer
tgaacccgtacaccgagtt

SEQ ID NO: 125 - DNA, 39 bp, Artificial Sequence
  HSV LAMP primer
agcggatctgctttcgcatggcgmgagagcctsctsagc

SEQ ID NO: 126 - DNA, 36 bp, Artificial Sequence
  HSV LAMP primer
cggatyccccagagcascccacsaccttgatggcgg

SEQ ID NO: 127 - DNA, 20 bp, Artificial Sequence
  HSV LAMP primer\
agccagtcscgcagsaggat

SEQ ID NO: 128 - DNA, 20 bp, Artificial Sequence
  HSV LAMP primer
gccgtsctcctsgacaagca

SEQ ID NO: 129 - DNA, 17 bp, Artificial Sequence
  HSV LAMP primer
ggctsgccatgcgaaag

SEQ ID NO: 130 - DNA, 17 bp, Artificial Sequence
  HSV LAMP primer
gtcgcgagsagcatctc

SEQ ID NO: 131 - DNA, 38 bp, Artificial Sequence
  HSV LAMP primer
tgcttgtcsaggagsacggcagatccgctcgcggatyc

SEQ ID NO: 132 - DNA, 38 bp, Artificial Sequence
  HSV LAMP primer
sgtgtgyaactcggtgtacgggtcacsgtsgcggcmac

SEQ ID NO: 133 - DNA, 19 bp, Artificial Sequence
  HSV LAMP primer
tcctcggggstgctctggg

SEQ ID NO: 134 - DNA, 17 bp, Artificial Sequence
  HSV LAMP primer
ttcacsggrgtgcagca

SEQ ID NO: 135 - DNA, 18 bp, Artificial Sequence
  HSV LAMP primer
gatggcgagccacatctc

SEQ ID NO: 136 - DNA, 20 bp, Artificial Sequence
  HSV LAMP primer
ggttgatraacgcgcagttg

SEQ ID NO: 137 - DNA, 40 bp, Artificial Sequence
  HSV LAMP primer
gcgatbagcagcagcttggtctgttyctscccccsatcaa

SEQ ID NO: 138 - DNA, 41 bp, Artificial Sequence
  HSV LAMP primer
gtacatcggcgtcatctrcgggttttgcgcaccagrtcsac

SEQ ID NO: 139 - DNA, 20 bp, Artificial Sequence
  HSV LAMP primer
gaacgtyttttcgcactcga

SEQ ID NO: 140 - DNA, 21 bp, Artificial Sequence
  HSV LAMP primer
ggyaagatgctcatyaagggc

SEQ ID NO: 141 - DNA, 18 bp, Artificial Sequence
  HSV LAMP primer
cccacytccgggtttcac

SEQ ID NO: 142 - DNA, 25 bp, Artificial Sequence
  HSV LAMP primer
gtgrgccttsacgaagaacavvvvv

SEQ ID NO: 143 - DNA, 41 bp, Artificial Sequence
  HSV LAMP primer
vvvvaggttgtgggcctggatgatgaccccgtggtggtgtt

SEQ ID NO: 144 - DNA, 40 bp, Artificial Sequence
  HSV LAMP primer
gtgcttcagyacgctctccccacctcgatctccaggtagt

SEQ ID NO: 145 - DNA, 20 bp, Artificial Sequence
  HSV LAMP primer
ggggtacaggctggcaaagt

SEQ ID NO: 146 - DNA, 19 bp, Artificial Sequence
  HSV LAMP primer
gtsgcgcacctggaggcgg

SEQ ID NO: 147 - DNA, 18 bp, Artificial Sequence
  HSV LAMP primer
gtgacrttcaaggccctg

SEQ ID NO: 148 - DNA, 17 bp, Artificial Sequence
  HSV LAMP primer
mgggacatcagcttcga

SEQ ID NO: 149 - DNA, 40 bp, Artificial Sequence
  HSV LAMP primer
gccacryytcgggaataaacctttggraataacgccaaga

SEQ ID NO: 150 - DNA, 43 bp, Artificial Sequence
  HSV LAMP primer
gcgctacggcggaggaaactctcatgctagagtatcaaaggct

SEQ ID NO: 151 - DNA, 19 bp, Artificial Sequence
  HSV LAMP primer
tttaacagactctcggtga

SEQ ID NO: 152 - DNA, 17 bp, Artificial Sequence
  HSV LAMP primer
cgtcgaatgttgcatag

SEQ ID NO: 153 - DNA, 17 bp, Artificial Sequence
  hAdv LAMP primer
cartggkcdtacatgca

SEQ ID NO: 154 - DNA, 16 bp, Artificial Sequence
  hAdv LAMP primer
gtgwascgmrcyttgt

SEQ ID NO: 155 - DNA, 36 bp, Artificial Sequence
  hAdv LAMP primer
aagtasgtrtckgtggcrcgcaggaygcytcggagt

SEQ ID NO: 156 - DNA, 34 bp, Artificial Sequence
  hAdv LAMP primer
acscacgatgtgaccaccgrtcmacgggsayraa

SEQ ID NO: 157 - DNA, 17 bp, Artificial Sequence
  hAdv LAMP primer
gcraactgcaccagmcc

SEQ ID NO: 158 - DNA, 17 bp, Artificial Sequence
  hAdv LAMP primer
cagcgdctgaygctgcg

SEQ ID NO: 159 - DNA, 17 bp, Artificial Sequence
  hAdv LAMP primer
cgatgmtgccscartgg

SEQ ID NO: 160 - DNA, 15 bp, Artificial Sequence
  hAdv LAMP primer
raascgcagcrtcag

SEQ ID NO: 161 - DNA, 37 bp, Artificial Sequence
  hAdv LAMP primer
aactgcaccagmccsggrccdtacatgcacatckcsg

SEQ ID NO: 162 - DNA, 37 bp, Artificial Sequence
  hAdv LAMP primer
cmgayacstacttcaryctgtcsgtggtcacatcgtg

SEQ ID NO: 163 - DNA, 20 bp, Artificial Sequence
  hAdv LAMP primer
aggtactccgargcrtcctg

SEQ ID NO: 164 - DNA, 19 bp, Artificial Sequence
  hAdv LAMP primer
aacaagtttagraacccca

SEQ ID NO: 165 - DNA, 16 bp, Artificial Sequence
  hAdv LAMP primer
ggctggtgcagttygc

SEQ ID NO: 166 - DNA, 18 bp, Artificial Sequence
  hAdv LAMP primer
cacrcggttrtcrcccac

SEQ ID NO: 167 - DNA, 36 bp, Artificial Sequence
  hAdv LAMP primer
tcgtgcgtrggygccaccgcgccaccgagasstact

SEQ ID NO: 168 - DNA, 38 bp, Artificial Sequence
  hAdv LAMP primer
ggtcycagcgyytgacgctgcggtraaccgcgcyttgt

SEQ ID NO: 169 - DNA, 25 bp, Artificial Sequence
  hAdv LAMP primer
gggttyctaaacttgttaytcaggc

SEQ ID NO: 170 - DNA, 19 bp, Artificial Sequence
  hAdv LAMP primer
ggttyatcccygtggaccg

SEQ ID NO: 171 - DNA, 18 bp, Artificial Sequence
  hAdv LAMP primer
tggckaccccwtcgatga

SEQ ID NO: 172 - DNA, 17 bp, Artificial Sequence
  hAdv LAMP primer
cgctgrgaccggtckgt

SEQ ID NO: 173 - DNA, 39 bp, Artificial Sequence
  hAdv LAMP primer
agcccggggctcaggtactcccgcagtggtcktacatgc

SEQ ID NO: 174 - DNA, 37 bp, Artificial Sequence
  hAdv LAMP primer
cgcgccaccgagasstactggtyacrtcgtgcgtrgg

SEQ ID NO: 175 - DNA, 20 bp, Artificial Sequence
  hAdv LAMP primer
cgaggcgtcctggcccgaga

SEQ ID NO: 176 - DNA, 20 bp, Artificial Sequence
  hAdv LAMP primer
gtttagraaccccacggtgg

SEQ ID NO: 177 - DNA, 17 bp, Artificial Sequence
  hAdv LAMP primer
gacctgggycaraacct

SEQ ID NO: 178 - DNA, 17 bp, Artificial Sequence
  hAdv LAMP primer
gttgccrgccgagaagg

SEQ ID NO: 179 - DNA, 40 bp, Artificial Sequence
  hAdv LAMP primer
ctcgtccatgggrtcsacctckctctaygcmaactccgcc

SEQ ID NO: 180 - DNA, 41 bp, Artificial Sequence
  hAdv LAMP primer
tygaagtctttgacgtggtccgggtacacggtytcgatgac

SEQ ID NO: 181 - DNA, 20 bp, Artificial Sequence
  hAdv LAMP primer
aargtcatrtckagcgcgtg

SEQ ID NO: 182 - DNA, 21 bp, Artificial Sequence
  hAdv LAMP primer
gtscaccagccgcaccgcggc

SEQ ID NO: 183 - DNA, 17 bp, Artificial Sequence
  hAdv LAMP primer
acgcacgaygtraccac

SEQ ID NO: 184 - DNA, 20 bp, Artificial Sequence
  hAdv LAMP primer
gtgccrgagtagggcttraa

SEQ ID NO: 185 - DNA, 38 bp, Artificial Sequence
  hAdv LAMP primer
gcrgtrtcctcrcggtccacgaccggtcycagcgyytg

SEQ ID NO: 186 - DNA, 40 bp, Artificial Sequence
  hAdv LAMP primer
tcgtacaargcgcggttyaccccgccgcgratgtcaaagt

SEQ ID NO: 187 - DNA, 18 bp, Artificial Sequence
  hAdv LAMP primer
rgggatraaccgcagcgt

SEQ ID NO: 188 - DNA, 19 bp, Artificial Sequence
  hAdv LAMP primer
gtgggygayaaccgygtgc

SEQ ID NO: 189 - DNA, 18 bp, Artificial Sequence
  hAdv LAMP primer
rtayytgcchgacaagct

SEQ ID NO: 190 - DNA, 17 bp, Artificial Sequence
  hAdv LAMP primer
gagcgrtarcgbaggcc

SEQ ID NO: 191 - DNA, 40 bp, Artificial Sequence
  hAdv LAMP primer
artcyachaycccvggrgccgtrraaathtctsmyaaccc

SEQ ID NO: 192 - DNA, 39 bp, Artificial Sequence
  hAdv LAMP primer
gctacatyaacctkggvgcrcgmgcattgcggtgrtggt

SEQ ID NO: 193 - DNA, 17 bp, Artificial Sequence
  hAdv LAMP primer
accactcgcttgttcat

SEQ ID NO: 194 - DNA, 23 bp, Artificial Sequence
  hAdv LAMP primer
gactayatggacaacgtyaaycc

SEQ ID NO: 195 - DNA, 19 bp, Artificial Sequence
  hAdv LAMP primer
gtkgacggrgcyagcatya

SEQ ID NO: 196 - DNA, 19 bp, Artificial Sequence
  hAdv LAMP primer
gggatrgakatgggsacgt

SEQ ID NO: 197 - DNA, 40 bp, Artificial Sequence
  hAdv LAMP primer
agcgtkgaggcsgtgttgtggabagcatytgyctytacgc

SEQ ID NO: 198 - DNA, 42 bp, Artificial Sequence
  hAdv LAMP primer
agraaygacaccaacgaccagtgggkatrggrtadagcatgt

SEQ ID NO: 199 - DNA, 20 bp, Artificial Sequence
  hAdv LAMP primer
ggccatsggraaraaggtgg

SEQ ID NO: 200 - DNA, 19 bp, Artificial Sequence
  V
gactaycthtccgccgcca

SEQ ID NO: 201 - DNA, 17 bp, Artificial Sequence
  hAdv LAMP primer
cgcctctgcgtgaagac

SEQ ID NO: 202 - DNA, 18 bp, Artificial Sequence
  hAdv LAMP primer
catytccaacgacctvgc

SEQ ID NO: 203 - DNA, 35 bp, Artificial Sequence
  hAdv LAMP primer
aggccgcsgtcaaygacacggcccggtgagcttga

SEQ ID NO: 204 - DNA, 40 bp, Artificial Sequence
  hAdv LAMP primer
cctgcacgtctccygagttgtcggacgcggagayctccag

SEQ ID NO: 205 - DNA, 20 bp, Artificial Sequence
  hAdv LAMP primer
attctgtcgaactctctttc

SEQ ID NO: 206 - DNA, 22 bp, Artificial Sequence
  hAdv LAMP primer
ggccatgaactgctcgatctct

SEQ ID NO: 207 - DNA, 16 bp, Artificial Sequence
  hAdv LAMP primer
csgcbaggtcgttgga

SEQ ID NO: 208 - DNA, 20 bp, Artificial Sequence
  hAdv LAMP primer
accaccctyaactacctctt

SEQ ID NO: 209 - DNA, 34 bp, Artificial Sequence
  hAdv LAMP primer
cagccgmgtctggaacgaratgcgsgccatgagc

SEQ ID NO: 210 - DNA, 37 bp, Artificial Sequence
  hAdv LAMP primer
cgcatgaccacctgcgcgatgcgmaactacgccgtct

SEQ ID NO: 211 - DNA, 20 bp, Artificial Sequence
  hAdv LAMP primer
mggmctcaacgccttctcgc

SEQ ID NO: 212 - DNA, 19 bp, Artificial Sequence
  hAdv LAMP primer
grttgagctccacgtgccg

SEQ ID NO: 213 - DNA, 16 bp, Artificial Sequence
  hAdv LAMP primer
gcgttgagkcckccct

SEQ ID NO: 214 - DNA, 20 bp, Artificial Sequence
  hAdv LAMP primer
gcgvcgctgggtyatgtact

SEQ ID NO: 215 - DNA, 36 bp, Artificial Sequence
  hAdv LAMP primer
tcgcgcaggtggtcatgcggttccagackcggctgt

SEQ ID NO: 216 - DNA, 39 bp, Artificial Sequence
  hAdv LAMP primer
agacggcgtagttkcgcaggtcttcgtggcmgarcacac

SEQ ID NO: 217 - DNA, 17 bp, Artificial Sequence
  hAdv LAMP primer
csargggggcgtggtct

SEQ ID NO: 218 - DNA, 20 bp, Artificial Sequence
  hAdv LAMP primer
ggtagttragggtggtggcg

SEQ ID NO: 219 - DNA, 17 bp, Artificial Sequence
  hAdv LAMP primer
sgcgaagacggcgtagt

SEQ ID NO: 220 - DNA, 16 bp, Artificial Sequence
  hAdv LAMP primer
tgtcggcgcgcaactc

SEQ ID NO: 221 - DNA, 40 bp, Artificial Sequence
  hAdv LAMP primer
tgcgvcgctgggtyatgtacttcgcaggcgctgraagagg

SEQ ID NO: 222 - DNA, 41 bp, Artificial Sequence
  hAdv LAMP primer
ttcgttgatrtcccccaaggccttcaacttcgccgtggact

SEQ ID NO: 223 - DNA, 20 bp, Artificial Sequence
  hAdv LAMP primer
accgccaccaccctyaacta

SEQ ID NO: 224 - DNA, 17 bp, Artificial Sequence
  hAdv LAMP primer
cgctccatggcctcgta

SEQ ID NO: 225 - DNA, 17 bp, Artificial Sequence
  hAdv LAMP primer
yctggargcggtggtsc

SEQ ID NO: 226 - DNA, 18 bp, Artificial Sequence
  hAdv LAMP primer
ccggtccargttggtctg

SEQ ID NO: 227 - DNA, 35 bp, Artificial Sequence
  hAdv LAMP primer
ggccctgttytcggccagcgckcraaccccacgca

SEQ ID NO: 228 - DNA, 38 bp, Artificial Sequence
  hAdv LAMP primer
sgaygaggccggsctggtstacacgttgcygctgttgt

SEQ ID NO: 229 - DNA, 17 bp, Artificial Sequence
  hAdv LAMP primer
tsgccagcaccttctcg

SEQ ID NO: 230 - DNA, 18 bp, Artificial Sequence
  hAdv LAMP primer
gctgctkcagcgcgtggc

SEQ ID NO: 231 - DNA, 16 bp, Artificial Sequence
  hAdv LAMP primer
atccgsccsgaygagg

SEQ ID NO: 232 - DNA, 19 bp, Artificial Sequence
  hAdv LAMP primer
tggtgtagtcctcctgycc

SEQ ID NO: 233 - DNA, 39 bp, Artificial Sequence
  hAdv LAMP primer
ccggtccargttggtctgcacgtstacgacgcgctgctk

SEQ ID NO: 234 - DNA, 36 bp, Artificial Sequence
  hAdv LAMP primer
agggcaacctgggctccattggcsggctgygtrctc

SEQ ID NO: 235 - DNA, 18 bp, Artificial Sequence
  hAdv LAMP primer
gttgtarcgrgccacgcg

SEQ ID NO: 236 - DNA, 22 bp, Artificial Sequence
  hAdv LAMP primer
tggtkgcrctraacgccttcct

SEQ ID NO: 237 - DNA, 20 bp, Artificial Sequence
  hAdv LAMP primer
ggtkgcrctraacgccttcc

SEQ ID NO: 238 - DNA, 19 bp, Artificial Sequence
  hAdv LAMP primer
cctggctcaggttyacsgt

SEQ ID NO: 239 - DNA, 39 bp, Artificial Sequence
  hAdv LAMP primer
gcagygcgctcacaaagttggttgagyacrcagccsgcc

SEQ ID NO: 240 - DNA, 41 bp, Artificial Sequence
  hAdv LAMP primer
ratggtgacygagacmccscartgcaggccytgtctrctgg

SEQ ID NO: 241 - DNA, 18 bp, Artificial Sequence
  hAdv LAMP primer
ctgyccccgcggcacgtt

SEQ ID NO: 242 - DNA, 22 bp, Artificial Sequence
  hAdv LAMP primer
accagtcsgggccrgactaytt

SEQ ID NO: 243 - DNA, 17 bp, Artificial Sequence
  hAdv LAMP primer
ccaactcgcgcctgytg

SEQ ID NO: 244 - DNA, 20 bp, Artificial Sequence
  hAdv LAMP primer
gttggtcagcaggtagttya

SEQ ID NO: 245 - DNA, 41 bp, Artificial Sequence
  hAdv LAMP primer
tckcggtacagkgtcagcargttgctgctratmgcgccstt

SEQ ID NO: 246 - DNA, 36 bp, Artificial Sequence
  hAdv LAMP primer
gccatmggkcaggcgcakgtgtgtcctcctgyccca

SEQ ID NO: 247 - DNA, 19 bp, Artificial Sequence
  hAdv LAMP primer
akgtgtcccgggacacgct

SEQ ID NO: 248 - DNA, 24 bp, Artificial Sequence
  hAdv LAMP primer
ggacgagcayacyttccaggagat

SEQ ID NO: 249 - DNA, 100 bp, Artificial Sequence
  Cas12b-crRNA scaffold template
gaaattaatacgactcactatagggtctagaggacagaatttttcaacgggtgtgccaatggccactttccaggtggcaaagcccgttgagcttctcaaa

SEQ ID NO: 250 - DNA, 55 bp, Artificial Sequence
  HSV crRNA template
accaccacggggtysacgtggtgccacttctcagatttgagaagctcaacgggct

SEQ ID NO: 251 - DNA, 55 bp, Artificial Sequence
  HSV crRNA template
caccaccacggggtysacgtgtgccacttctcagatttgagaagctcaacgggct

SEQ ID NO: 252 - DNA, 55 bp, Artificial Sequence
  HSV crRNA template
gggtacaggctggcaaagtcgtgccacttctcagatttgagaagctcaacgggct

SEQ ID NO: 253 - DNA, 55 bp, Artificial Sequence
  HSV crRNA template
atgctggggtacaggctggcgtgccacttctcagatttgagaagctcaacgggct

SEQ ID NO: 254 - DNA, 55 bp, Artificial Sequence
  HSV crRNA template
gatgctggggtacaggctgggtgccacttctcagatttgagaagctcaacgggct

SEQ ID NO: 255 - DNA, 55 bp, Artificial Sequence
  HSV crRNA template
cccagcatcatccaggcccagtgccacttctcagatttgagaagctcaacgggct

SEQ ID NO: 256 - DNA, 55 bp, Artificial Sequence
  HSV crRNA template
gscckcagggagagcgtrctgtgccacttctcagatttgagaagctcaacgggct

SEQ ID NO: 257 - DNA, 55 bp, Artificial Sequence
  HSV crRNA template
ggscgacggctgttcttcgtgtgccacttctcagatttgagaagctcaacgggct

SEQ ID NO: 258 - DNA, 55 bp, Artificial Sequence
  hAdv crRNA template
tacctctttcagcgcctgcggtgccacttctcagatttgagaagctcaacgggct

SEQ ID NO: 259 - DNA, 55 bp, Artificial Sequence
  hAdv crRNA template
gaacacaccgccaccaccctgtgccacttctcagatttgagaagctcaacgggct

SEQ ID NO: 260 - DNA, 55 bp, Artificial Sequence
  hAdv crRNA template
ttatgtacttcttcgtggcagtgccacttctcagatttgagaagctcaacgggct

SEQ ID NO: 261 - DNA, 55 bp, Artificial Sequence
  hAdv crRNA template
gtggcggtgtgttctgccacgtgccacttctcagatttgagaagctcaacgggct

SEQ ID NO: 262 - DNA, 55 bp, Artificial Sequence
  hAdv crRNA template
gcggtgtgttctgccacgaagtgccacttctcagatttgagaagctcaacgggct

SEQ ID NO: 263 - DNA, 55 bp, Artificial Sequence
  hAdv crRNA template
ttctgccacgaagaagtacagtgccacttctcagatttgagaagctcaacgggct

SEQ ID NO: 264 - DNA, 55 bp, Artificial Sequence
  hAdv crRNA template
aagtacataacccagcgycggtgccacttctcagatttgagaagctcaacgggct

SEQ ID NO: 265 - DNA, 55 bp, Artificial Sequence
  hAdv crRNA template
aggccttgggggatatcaacgtgccacttctcagatttgagaagctcaacgggct

SEQ ID NO: 266 - DNA, 55 bp, Artificial Sequence
  hAdv crRNA template
cttgaggccttgggggatatgtgccacttctcagatttgagaagctcaacgggct

SEQ ID NO: 267 - DNA, 55 bp, Artificial Sequence
  hAdv crRNA template
gtggattcgttgatatccccgtgccacttctcagatttgagaagctcaacgggct

SEQ ID NO: 268 - DNA, 55 bp, Artificial Sequence
  hAdv crRNA template
gttgatatcccccaaggcctgtgccacttctcagatttgagaagctcaacgggct

SEQ ID NO: 269 - DNA, 55 bp, Artificial Sequence
  hAdv crRNA template
gcctcgtagaagtccacggcgtgccacttctcagatttgagaagctcaacgggct

SEQ ID NO: 270 - DNA, 55 bp, Artificial Sequence
  hAdv crRNA template
gcgcgcaactcccagtttttgtgccacttctcagatttgagaagctcaacgggct

SEQ ID NO: 271 - DNA, 55 bp, Artificial Sequence
  hAdv crRNA template
tagaagtccacggcgaagttgtgccacttctcagatttgagaagctcaacgggct

SEQ ID NO: 272 - DNA, 55 bp, Artificial Sequence
  hAdv crRNA template
agaagtccacggcgaagttggtgccacttctcagatttgagaagctcaacgggct

SEQ ID NO: 273 - DNA, 55 bp, Artificial Sequence
  hAdv crRNA template
gaagtccacggcgaagttgagtgccacttctcagatttgagaagctcaacgggct

SEQ ID NO: 274 - DNA, 55 bp, Artificial Sequence
  hAdv crRNA template
aagtccacggcgaagttgaagtgccacttctcagatttgagaagctcaacgggct

SEQ ID NO: 275 - DNA, 18 bp, Artificial Sequence
  human ACTB LAMP primer
agtaccccatcgagcacg

SEQ ID NO: 276 - DNA, 20 bp, Artificial Sequence
  human ACTB LAMP primer
agcctggatagcaacgtaca

SEQ ID NO: 277 - DNA, 40 bp, Artificial Sequence
  human ACTB LAMP primer
gagccacacgcagctcattgtatcaccaactgggacgaca

SEQ ID NO: 278 - DNA, 38 bp, Artificial Sequence
  human ACTB LAMP primer
ctgaaccccaaggccaaccggctggggtgttgaaggtc

SEQ ID NO: 279 - DNA, 21 bp, Artificial Sequence
  human ACTB LAMP primer
tgtggtgccagattttctcca

SEQ ID NO: 280 - DNA, 23 bp, Artificial Sequence
  human ACTB LAMP primer
cgagaagatgacccagatcatgt

SEQ ID NO: 281 - DNA, 40 bp, Artificial Sequence; identical to SEQ ID NO: 277
  human ACTB LAMP probe

SEQ ID NO: 282 - DNA, 22 bp, Artificial Sequence
  human ACTB LAMP probe
tacaatgagctgcgtgtggctc

SEQ ID NO: 283 - DNA, 22 bp, Artificial Sequence
  SARS-CoV-2 S LAMP primer
tcttacctttcttttccaatgt

SEQ ID NO: 284 - DNA, 19 bp, Artificial Sequence
  SARS-CoV-2 S LAMP primer
aaatggtaggacagggtta

SEQ ID NO: 285 - DNA, 25 bp, Artificial Sequence
  SARS-CoV-2 S LAMP primer
aagtctgtgaatttcaattttgtaa

SEQ ID NO: 286 - DNA, 21 bp, Artificial Sequence
  SARS-CoV-2 S LAMP primer
gagagacatattcaaaagtgc

SEQ ID NO: 287 - DNA, 45 bp, Artificial Sequence
  SARS-CoV-2 S LAMP primer
tggaagcaaaataaacaccatcatttctctgggaccaatgktayt

SEQ ID NO: 288 - DNA, 50 bp, Artificial Sequence
  SARS-CoV-2 S LAMP primer
attgttaataacgctactaatgttgttatcaacttttgttgtttttgtgg

SEQ ID NO: 289 - DNA, 46 bp, Artificial Sequence
  SARS-CoV-2 alpha mutant S LAMP probe
tggaagcaaaataaacaccatcatttacttggttccatgctatctc

SEQ ID NO: 290 - DNA, 46 bp, Artificial Sequence
  SARS-CoV-2 beta mutant S LAMP probe
tggaagcaaaataaacaccatcattcaatggtactaagaggtttcc

SEQ ID NO: 291 - DNA, 25 bp, Artificial Sequence
  SARS-CoV-2 S LAMP probe
aatgatggtgtttattttgcttcca

SEQ ID NO: 292 - DNA, 23 bp, Artificial Sequence
  E1-F3
tgagtacgaacttatgtactcat

SEQ ID NO: 293 - DNA, 22 bp, Artificial Sequence
  E1-B3
ttcagatttttaacacgagagt

SEQ ID NO: 294 - DNA, 42 bp, Artificial Sequence
  E1-FIP
accacgaaagcaagaaaaagaagttcgtttcggaagagacag

SEQ ID NO: 295 - DNA, 44 bp, Artificial Sequence
  E1-BIP
ttgctagttacactagccatccttaggttttacaagactcacgt

SEQ ID NO: 296 - DNA, 18 bp, Artificial Sequence
  E1-LF
cgctattaactattaacg

SEQ ID NO: 297 - DNA, 19 bp, Artificial Sequence
  E1-LB
gcgcttcgattgtgtgcgt

SEQ ID NO: 298 - DNA, 42 bp, Artificial Sequence; identical to SEQ ID NO: 294
  E1-FIP-HEX

SEQ ID NO: 299 - DNA, 24 bp, Artificial Sequence
  E1-F1-FQ
acttctttttcttgctttcgtggt

SEQ ID NO: 300 - DNA, 18 bp, Artificial Sequence
  Orf1a_F3
cggtggacaaattgtcac

SEQ ID NO: 301 - DNA, 22 bp, Artificial Sequence
  Orf1a_B3
cttctctggatttaacacactt

SEQ ID NO: 302 - DNA, 28 bp, Artificial Sequence
  Orf1a_LF
ttacaagcttaaagaatgtctgaacact

SEQ ID NO: 303 - DNA, 27 bp, Artificial Sequence
  Orf1a_LB
ttgaatttaggtgaaacatttgtcacg

SEQ ID NO: 304 - DNA, 47 bp, Artificial Sequence
  Orf1a_FIP
tcagcacacaaagccaaaaatttatctgtgcaaaggaaattaaggag

SEQ ID NO: 305 - DNA, 45 bp, Artificial Sequence
  Orf1a_BIP
tattggtggagctaaacttaaagccctgtacaatccctttgagtg

SEQ ID NO: 306 - DNA, 47 bp, Artificial Sequence; identical to SEQ ID NO: 304
  Orf1a_FIP-CY5

SEQ ID NO: 307 - DNA, 25 bp, Artificial Sequence
  Orf1a_F1C-RQ
ataaatttttggctttgtgtgctga

SEQ ID NO: 308 - DNA, 121 bp, Artificial Sequence
  Concensus EBNA-1 gene
gtcgccggtgtgttcgtatatggaggtagtaagacctccctttacaacctcaggcgaggaattgcccttgctattccacaatgtcgtattacaccattgagtcgtctcccctttggaatgg

SEQ ID NO: 309 - DNA, 121 bp, Artificial Sequence
  NA-1 gene from  Human herpesvirus 4 strain YCCEL1
gtcgccggtgtgttcgtatatggaggtagtaagacctccctttacaacctcaggcgaggaattgcccttgctgttccacaatgtcgtattacaccattgagtcgtctcccctttggaatgg

SEQ ID NO: 310 - DNA, 121 bp, Artificial Sequence; identical to SEQ ID NO: 309
  EBNA-1 gene from  Human herpesvirus 4 strain GD1

SEQ ID NO: 311 - DNA, 121 bp, Artificial Sequence; identical to SEQ ID NO: 309
  EBNA-1 gene from  Human herpesvirus 4 strain GD2

SEQ ID NO: 312 - DNA, 121 bp, Artificial Sequence; identical to SEQ ID NO: 309
  EBNA-1 gene from  Human herpesvirus 4 strain HKNPC1

SEQ ID NO: 313 - DNA, 121 bp, Artificial Sequence; identical to SEQ ID NO: 309
  EBNA-1 gene from  Human herpesvirus 4 strain Akata

SEQ ID NO: 314 - DNA, 121 bp, Artificial Sequence
  EBNA-1 gene from  Human herpesvirus 4 strain Mutu
gtcgccggtgtgttcgtatatggaggtagtaagacctccctttacaacctcaggcgaggaattggccttgctattccacaatgtcgtcttacaccattgagtcgtctcccctttggaatgg

SEQ ID NO: 315 - DNA, 121 bp, Artificial Sequence; identical to SEQ ID NO: 314
  EBNA-1 gene from  Human herpesvirus 4 strain K4123-Mi

SEQ ID NO: 316 - DNA, 121 bp, Artificial Sequence
  EBNA-1 gene from  Human herpesvirus 4 strain K4123-MiEBV
gtcgccggtgtgttcgtatatggaggtagtaagacctccctttacaacctcaggcgaggaattgcccttgctattccacaatgtcgtcttacaccattgagtcgtctcccctttggaatgg

SEQ ID NO: 317 - DNA, 121 bp, Artificial Sequence; identical to SEQ ID NO: 309
  EBNA-1 gene from  Human herpesvirus 4 strain C666-1

SEQ ID NO: 318 - DNA, 121 bp, Artificial Sequence; identical to SEQ ID NO: 309
  EBNA-1 gene from  Human herpesvirus 4 strain M81

SEQ ID NO: 319 - DNA, 121 bp, Artificial Sequence; identical to SEQ ID NO: 314
  EBNA-1 gene from  Human herpesvirus 4 strain Raji

SEQ ID NO: 320 - DNA, 121 bp, Artificial Sequence; identical to SEQ ID NO: 309
  EBNA-1 gene from  Human herpesvirus 4 strain GC1

SEQ ID NO: 321 - DNA, 121 bp, Artificial Sequence; identical to SEQ ID NO: 314
  EBNA-1 gene from  Human herpesvirus 4 strain Jijoye

SEQ ID NO: 322 - DNA, 121 bp, Artificial Sequence
  EBNA-1 gene from  Human herpesvirus 4
gtcgccggtgtgttcgtatatggaggtagtaagacctccctttacaacctaaggcgaggaactgcccttgctattccacaatgtcgtcttacaccattgagtcgtctcccctttggaatgg

SEQ ID NO: 323 - DNA, 121 bp, Artificial Sequence; identical to SEQ ID NO: 314
  EBNA-1 gene from  Human herpesvirus 4

SEQ ID NO: 324 - DNA, 121 bp, Artificial Sequence; identical to SEQ ID NO: 322
  EBNA-1 gene from  Epstein-Barr virus strain B95-8

SEQ ID NO: 325 - DNA, 106 bp, Artificial Sequence
  Concensus BamHIW gene
ccactgcccctggtataaagtggtcctgcagctatttctggtcgcatcagagcgccaggagtccacacaaatgtaagagggggtcttctacctctccctagccctc

SEQ ID NO: 326 - DNA, 106 bp, Artificial Sequence; identical to SEQ ID NO: 325
  BamHIW gene from  Human herpesvirus 4 strain YCCEL1

SEQ ID NO: 327 - DNA, 106 bp, Artificial Sequence; identical to SEQ ID NO: 325
  BamHIW gene from  Human herpesvirus 4 strain GD1

SEQ ID NO: 328 - DNA, 106 bp, Artificial Sequence; identical to SEQ ID NO: 325
  BamHIW gene from  Human herpesvirus 4 strain GD2

SEQ ID NO: 329 - DNA, 106 bp, Artificial Sequence; identical to SEQ ID NO: 325
  BamHIW gene from  Human herpesvirus 4 strain HKNPC1

SEQ ID NO: 330 - DNA, 106 bp, Artificial Sequence; identical to SEQ ID NO: 325
  BamHIW gene from  Human herpesvirus 4 strain Akata

SEQ ID NO: 331 - DNA, 106 bp, Artificial Sequence; identical to SEQ ID NO: 325
  BamHIW gene from  Human herpesvirus 4 strain Mutu

SEQ ID NO: 332 - DNA, 106 bp, Artificial Sequence; identical to SEQ ID NO: 325
  BamHIW gene from  Human herpesvirus 4 strain K4123-Mi

SEQ ID NO: 333 - DNA, 106 bp, Artificial Sequence; identical to SEQ ID NO: 325
  BamHIW gene from  Human herpesvirus 4 strain K4123-MiEBV

SEQ ID NO: 334 - DNA, 106 bp, Artificial Sequence; identical to SEQ ID NO: 325
  BamHIW gene from  Human herpesvirus 4 strain M81

SEQ ID NO: 335 - DNA, 106 bp, Artificial Sequence; identical to SEQ ID NO: 325
  BamHIW gene from  Human herpesvirus 4 strain Raji

SEQ ID NO: 336 - DNA, 106 bp, Artificial Sequence; identical to SEQ ID NO: 325
  BamHIW gene from  Human herpesvirus 4 strain GC1

SEQ ID NO: 337 - DNA, 106 bp, Artificial Sequence; identical to SEQ ID NO: 325
  BamHIW gene from  Human herpesvirus 4

SEQ ID NO: 338 - DNA, 106 bp, Artificial Sequence; identical to SEQ ID NO: 325
  BamHIW gene from  Human herpesvirus 4

SEQ ID NO: 339 - DNA, 106 bp, Artificial Sequence; identical to SEQ ID NO: 325
  BamHIW gene from  Epstein-Barr virus strain B95-8